(19) United States
(12) Patent Application Publication
(10) Pub. No.: US 2021/0330684 A1
(43) Pub. Date: Oct. 28, 2021

(54) AZD3355 (LESOGABERAN) FOR TREATMENT AND PREVENTION OF NONALCOHOLIC STEATOHEPATITIS (NASH), LIVER FIBROSIS, AND OTHER LIVER CONDITIONS

(71) Applicant: Icahn School of Medicine at Mount Sinai, New York, NY (US)

(72) Inventors: Joel DUDLEY, New York, NY (US); Scott FRIEDMAN, New York, NY (US); Benjamin READHEAD, New York, NY (US); Christine BECKER, New York, NY (US); Dipankar BHATTACHARYA, New York, NY (US)

(21) Appl. No.: 17/284,161

(22) PCT Filed: Oct. 11, 2019
(86) PCT No.: PCT/US19/55800

§ 371 Date: Apr. 9, 2021

Related U.S. Application Data
(60) Provisional application No. 62/744,927, filed on Oct. 12, 2018.

(51) Int. Cl. A61K 31/662; A61K 45/06; A61P 1/16

(52) U.S. Cl. CPC A61K 31/662; A61P 1/16; A61K 45/06

(57) ABSTRACT

The present invention relates to methods of treatment or prevention of fatty liver disease, nonalcoholic fatty liver disease (NAFLD) including nonalcoholic steatohepatitis (NASH), cirrhosis, liver fibrosis, hepatocellular carcinoma and related liver disease and conditions by administering an effective amount of a GABA_{B} agonist, lesogaberan (AZD3355), or related compounds.

### CROSS REFERENCE TO RELATED APPLICATION

The present application claims priority to U.S. Patent Application Ser. No. 62/744,927 filed Oct. 12, 2018, which is incorporated by reference as if expressly set forth in its entirety herein.

### FIELD OF THE INVENTION

The present disclosure relates to methods of treatment or prevention of nonalcoholic steatohepatitis (NASH), as well as liver fibrosis of other liver etiologies, by administering a therapeutically effective amount of lesogaberan (AZD3355) or related compounds.

### BACKGROUND OF THE INVENTION

Nonalcoholic steatohepatitis (NASH) is a condition that causes inflammation, accumulation of fat and fibrous (scar) tissue in the liver. NASH has emerged as the leading cause of chronic liver disease worldwide. Liver enzyme levels in the blood may be more elevated than the mild elevations seen with nonalcoholic fatty liver disease (NAFLD). Although a similar condition can occur in people who abuse alcohol, NASH occurs in those who drink little to no alcohol. The exact cause of NASH is unknown. However, it is seen more frequently in people with certain medical conditions such as diabetes, obesity, and insulin resistance. This combination of disorders is often called the metabolic syndrome.

It is not clear how many people have NASH as symptoms are often unnoticed or mild until it advances to cirrhosis. However, NASH is diagnosed in about 3 to 5 percent of people in the United States via liver biopsy. Most subjects with NASH are between the ages of 40 and 60 years, although the condition can also occur in children over the age of 10 years. NASH is seen more often in women than in men.

The cause of NASH is not clear, although research is ongoing in an attempt to find effective treatments. At the present time, treatment of NASH focuses on controlling some of the medical conditions associated with it (such as diabetes and obesity) and monitoring for progression. Currently, there are no effective treatments for NASH and related liver conditions and there is an urgent need for new therapeutics.

Beyond NASH, liver fibrosis can result from a number of other causes including infection (viral, bacterial or parasitic), drug-induced liver injury, enzyme deficiency, storage disorders, lipid abnormalities, and alcohol abuse. Treatment is focused on removing the insult if known, possible and the injury-induced remodeling is not too advanced. As such is not frequently successful, new therapies that prevent the progression and/or promote the resolution of liver fibrosis are needed.

### SUMMARY OF THE INVENTION

In certain embodiments, the present disclosure provides for a method for treating or preventing a liver disease or condition in a subject in need thereof, comprising administering to the subject, a therapeutically effective amount of a peripheral acting GABA_{B} agonist.

In certain embodiments, the liver condition includes but is not limited to fatty liver disease, nonalcoholic fatty liver disease (NAFLD), adiposity, liver fibrosis, cirrhosis, hepatocellular carcinoma, and combinations thereof.

In certain embodiments, the nonalcoholic fatty liver disease is nonalcoholic steatosis hepatitis or nonalcoholic steatohepatitis (NASH). In certain embodiments, the fatty liver disease is steatosis hepatitis or steatohepatitis.

In certain embodiments, the subject has one or more symptoms including but not limited to hepatic inflammation, hepatocyte injury or death, insulin resistance, weight gain, dyslipidemia, and fibrosis.

In some embodiments, the administration of the peripheral acting GABA_{B} agonist causes any one or combination of these symptoms to decrease in the subject.

In certain embodiments, the liver fibrosis or cirrhosis is associated with or due to fatty liver disease, nonalcoholic fatty liver disease, liver inflammation, hepatocyte injury or death, adiposity, hepatocellular carcinoma, and any combination thereof.

In certain embodiments, the liver fibrosis is a result of alcohol use, infection including viral, bacterial or parasitic, or immune mediated disorders.

In certain embodiments, the peripheral acting GABA_{B} agonist is AZD3355 (lesogaberan), or a pharmaceutically acceptable salt thereof.

In certain embodiments, the composition is administered twice daily.

In certain embodiments, the subject is a mammal. In certain embodiments, the subject is a human patient.

In further embodiments, the present disclosure provides for a method of inhibiting liver fibrosis in a subject in need thereof comprising administering a therapeutically effective amount of GABA_{B} agonist, or a pharmaceutically acceptable salt thereof, to the subject.

In certain embodiments, the liver fibrosis is associated with nonalcoholic steatosis hepatitis or nonalcoholic steatohepatitis (NASH). In certain embodiments, the liver fibrosis is associated with steatosis hepatitis or steatohepatitis. In certain embodiments, the liver fibrosis is associated with or due to fatty liver disease, adiposity, liver inflammation, hepatocyte injury or death, hepatocellular carcinoma, and combinations thereof.

In certain embodiments, the subject has one or more symptoms including but not limited to hepatic inflammation, hepatocyte injury or death, insulin resistance, weight gain, dyslipidemia, and fibrosis.

In some embodiments, the administration of the peripheral acting GABA_{B} agonist causes any one or combination of these symptoms to decrease in the subject.

In certain embodiments, the GABA_{B} agonist is AZD3355 (lesogaberan), or a pharmaceutically acceptable salt thereof.

In certain embodiments, the composition is administered twice daily.

In certain embodiments, the subject is a mammal. In certain embodiments, the subject is a human patient.

In certain embodiments, the present disclosure relates to a method for increasing GABA_{B} activity in a hepatocyte comprising contacting the hepatocyte with AZD3355, or a pharmaceutically acceptable salt thereof.

### BRIEF DESCRIPTION OF THE DRAWINGS

For the purpose of illustrating the invention, there are depicted in drawings certain embodiments of the invention. However, the invention is not limited to the precise arrangements and instrumentalities of the embodiments depicted in the drawings.

FIG. 1 shows MTS assay results for cell cytotoxicity in LX-2 cells treated with vehicle control or AZD3355. FIG. 1A shows LX-2 cells treated with 0-300 nM for 24 hours. FIG. 1B shows LX-2 cells treated with 0-300 nM for 48 hours. FIG. 1C shows LX-2 cells treated with 0-300 nM for 72 hours.

FIG. 2 shows MTS assay results for cell cytotoxicity in phHSCs treated with vehicle control or 30 or 100 nM AZD3355 for 48 hours (FIG. 2A) or 72 hours (FIG. 2B).

FIG. 3 shows LX-2 cell proliferation after being exposed to vehicle control or 0-100 nM of AZD3355 for 24 hours (FIGS. 3A and 3D), 48 hours (FIGS. 3B and 3E) or 72 hours (FIGS. 3C and 3F). FIGS. 3A, 3B, and 3C show absorbance and FIGS. 3D, 3E, and 3F show cell proliferation as a percent of vehicle control.

FIG. 4 shows phHSCs cell proliferation after being exposed to vehicle control or 30 or 100 nM of AZD3355 for 48 hours (FIGS. 4A and 4C) or 72 hours (FIGS. 4B and 4D). FIGS. 4A and 4B show absorbance and FIGS. 4C and 4D show cell proliferation as a percent of vehicle control.

FIG. 5 shows the cell apoptotic effects on LX-2 cells, as measured by caspase-3/7 activity, exposed to vehicle control or either 30 or 100 nM of AZD3355 for 72 hours. DMSO (3%) was used as the apoptotic positive control.

FIG. 6 shows the cell apoptotic effects on phHSCs, as measured by caspase-3/7 activity, exposed to vehicle control or either 30 or 100 nM of AZD3355 for 72 hours. DMSO (3%) was used as the apoptotic positive control.

FIG. 7 shows mRNA expression levels of genes as measured by qPCR in LX-2 cells after treatment with vehicle control or 30 or 100 nM of AZD3355 for 48 or 72 hours. FIG. 7A shows the expression of GAPDH. FIG. 7B shows the expression of RPII. FIG. 7C shows the expression of tubulin. FIG. 7D shows the expression of β-actin.

FIG. 8 shows relative mRNA expression levels of pro-fibrogenic genes as measured by qPCR in LX-2 cells after treatment with vehicle control or 30 or 100 nM of AZD3355 or 7.5 μM of sorafenib for 48 hours. The black bar shows downregulation by the treatment and the gray bars shows the rescue of the genes after withdrawal of the drug at 48 hours and the maintenance of the cells in drug free media for an additional 48 hours indicating that the drug induction is not toxic (*=p<0.05). FIG. 8A shows Col1α1 expression. FIG. 8B shows αSMA expression. FIG. 8C shows βPDGF-R expression. FIG. 8D shows TGFβ-R1 expression. FIG. 8E shoes TIMP1 expression. FIG. 8F shows TIMP2 expression. FIG. 8G shows MMP2 expression.

FIG. 9 shows relative mRNA expression levels of pro-fibrogenic genes as measured by qPCR in LX-2 cells after treatment with vehicle controls or 30 or 100 nM of AZD3355 or 7.5 μM of sorafenib for 72 hours. The black bar shows downregulation by the treatment and the gray bars shows the rescue of the genes after withdrawal of the drug at 72 hours and the maintenance of the cells in drug free media for an additional 72 hours indicating that the drug induction is not toxic (*=p<0.05). FIG. 9A shows Col1α1 expression. FIG. 9B shows αSMA expression. FIG. 9C shows βPDGF-R expression. FIG. 9D shows TGFβ-R1 expression. FIG. 9E shoes TIMP1 expression. FIG. 9F shows TIMP2 expression. FIG. 9G shows MMP2 expression.

FIG. 10 shows mRNA expression levels of genes as measured by qPCR in phHSCs after treatment with vehicle control or 30 or 100 nM of AZD3355 for 48 or 72 hours. FIG. 10A shows the mRNA expression of GAPDH. FIG. 10B shows the expression of RPII. FIG. 10C shows the expression of tubulin. FIG. 10D shows the expression of β-actin. FIG. 10E shows RPL13A.

FIG. 11 shows relative mRNA expression levels of pro-fibrogenic genes as measured by qPCR in phHSCs after treatment with vehicle control or 30 or 100 nM of AZD3355 or 7.5 μM of sorafenib for 48 hours showing that the expression of βPDGF-R, TGFβ-R1 and TIMP1 was downregulated compared to a control at 30 nM of AZD3355 treatment. FIG. 11A shows Col1α1 expression. FIG. 11B shows αSMA expression. FIG. 11C shows βPDGF-R expression. FIG. 11D shows TGFβ-R1 expression. FIG. 11E shoes TIMP1 expression. FIG. 11F shows TIMP2 expression. FIG. 11G shows MMP2 expression.

FIG. 12 shows relative mRNA expression levels of pro-fibrogenic genes as measured by qPCR in phHSCs after treatment with vehicle control or 30 or 100 nM of AZD3355 or 7.5 μM of sorafenib for 72 hours showing that the expression of the genes except TIMP1 and TIMP2 was downregulated compared to a control at 30 nM of AZD3355.

FIG. 12A shows Col1α1 expression. FIG. 12B shows αSMA expression. FIG. 12C shows βPDGF-R expression. FIG. 12D shows TGFβ-R1 expression. FIG. 12E shoes TIMP1 expression. FIG. 12F shows TIMP2 expression. FIG. 12G shows MMP2 expression.

FIG. 13 show expression levels of Col1α1 protein in LX-2 cells after treatment with vehicle control or 30 or 100 nM of AZD3355 or 7.5 μM of sorafenib for 48 hours. FIG. 13A is a representative western blot of total cell lysates. FIG. 13B is a graph of the relative expression of protein as a percent of vehicle control. FIG. 13C is the relative protein expression of the secreted protein in culture medium as detected by ELISA.

FIG. 14 show expression levels of MMP and αSMA protein in LX-2 cells after treatment with vehicle control or 30 or 100 nM of AZD3355 or 7.5 μM of sorafenib for 48 hours. FIG. 14A is a representative western blot of total cell lysates. FIG. 14B is a graph of the relative expression of MMP2 as a percent of vehicle control. FIG. 14C is a graph of the relative expression of αSMA as a percent of vehicle control.

FIG. 15 show expression levels of Col1α1 protein in LX-2 cells after treatment with vehicle control or 30 or 100 nM of AZD3355 or 7.5 μM of sorafenib for 72 hours. FIG. 15A is a representative western blot of total cell lysates. FIG. 15B is a graph of the relative expression of protein as a percent of vehicle control. FIG. 15C is the relative protein expression of the secreted protein in culture medium as detected by ELISA.

FIG. 16 show expression levels of MMP and αSMA protein in LX-2 cells after treatment with vehicle control or 30 or 100 nM of AZD3355 or 7.5 μM of sorafenib for 72 hours. FIG. 16A is a representative western blot of total cell lysates. FIG. 16B is a graph of the relative expression of MMP2 as a percent of vehicle control. FIG. 16C is a graph of the relative expression of αSMA as a percent of vehicle control.

FIG. 17 show expression levels of Col1α1 protein in phHSCs after treatment with vehicle control or 30 or 100 nM of AZD3355 or 7.5 μM of sorafenib for 48 hours. FIG. 17A is a representative western blot of total cell lysates. FIG. 17B is a graph of the relative expression of protein as a percent of vehicle control. FIG. 17C is the relative protein expression of the secreted protein in culture medium as detected by ELISA.

FIG. 18 show expression levels of MMP and αSMA protein in phHSCs after treatment with vehicle control or 30 or 100 nM of AZD3355 or 7.5 μM of sorafenib for 48 hours. FIG. 18A is a representative western blot of total cell lysates. FIG. 18B is a graph of the relative expression of MMP2 as a percent of vehicle control. FIG. 18C is a graph of the relative expression of αSMA as a percent of vehicle control.

FIG. 19 show expression levels of Col1α1 protein in phHSCs after treatment with vehicle control or 30 or 100 nM of AZD3355 or 7.5 μM of sorafenib for 72 hours. FIG. 19A is a representative western blot of total cell lysates. FIG. 19B is a graph of the relative expression of protein as a percent of vehicle control. FIG. 19C is the relative protein expression of the secreted protein in culture medium as detected by ELISA.

FIG. 20 show expression levels of MMP and αSMA protein in phHSCs after treatment with vehicle control or 30 or 100 nM of AZD3355 or 7.5 μM of sorafenib for 72 hours. FIG. 20A is a representative western blot of total cell lysates. FIG. 20B is a graph of the relative expression of MMP2 as a percent of vehicle control. FIG. 20C is a graph of the relative expression of αSMA as a percent of vehicle control.

FIG. 21 shows the immunocytochemistry of αSMA protein expression in LX-2 cells. Cells were exposed to vehicle control or either 30 or 100 nM of AZD3355 or 7.5 μM of sorafenib for 48 hours (FIG. 21A) or 72 hours (FIG. 21B) and immunostained with αSMA antibody. Recused expression of αSMA protein compared to vehicle control was observed with AZD3355 treatment.

FIG. 22 shows the immunocytochemistry of αSMA protein expression in phHSCs. Cells were exposed to vehicle control or either 30 or 100 nM of AZD3355 or 7.5 μM of sorafenib for 48 hours (FIG. 22A) or 72 hours (FIG. 22B) and immunostained with αSMA antibody. Reduced expression of αSMA protein compared to vehicle control was observed with AZD3355 treatment.

FIG. 23 shows representative images of human liver slices stained with H&E after treatment with differing concentrations of AZD3355 or sorafenib for 24 hours showing that the liver cells viability did not change across AZD3355 or sorafenib treatment.

FIG. 24 shows relative gene expression as measured by qPCR in various samples of human liver slices after treatment with the indicated concentrations of AZD3355 and sorafenib for 24 hours. FIG. 24A shows expression of Col1α1 in sample AZ1. FIG. 24B shows expression of TNF-α in sample AZ1. FIG. 24C shows IL-6 expression in sample AZ1. FIG. 24D shows expression of Col1α1 in sample AZ2. FIG. 24E shows expression of TNF-α in sample AZ2. FIG. 24F shows IL-6 expression in sample AZ2. FIG. 24G shows expression of Col1α1 in sample AZ3. FIG. 24H shows expression of TNF-α in sample AZ3. FIG. 24I shows IL-6 expression in sample AZ3. FIG. 24J shows expression of Col1α1 in sample AZ4. FIG. 24K shows expression of TNF-α in sample AZ4. FIG. 24L shows IL-6 expression in sample AZ4. FIG. 24M shows expression of Col1α1 in sample AZ5. FIG. 24N shows expression of TNF-α in sample AZ5. FIG. 24O shows IL-6 expression in sample AZ5. FIG. 24P shows expression of Col1α1 in sample AZ6. FIG. 24Q shows expression of TNF-α in sample AZ6. FIG. 24R shows IL-6 expression in sample AZ6. FIG. 24S shows expression of Col1α1 in sample AZ7. FIG. 24T shows expression of TNF-α in sample AZ7. FIG. 24U shows IL-6 expression in sample AZ7. *=p<0.05.

FIG. 25 shows relative gene expression as measured by qPCR in various additional samples of human liver slices after treatment with the indicated concentrations of AZD3355 for 24 hours. FIG. 25A shows expression of Col1α1 in sample ev417. FIG. 25B shows expression of TNF-α in sample ev417. FIG. 25C shows IL-6 expression in sample ev417. FIG. 25D shows expression of Col1α1 in sample ev422. FIG. 25E shows expression of TNF-α in sample ev422. FIG. 25F shows IL-6 expression in sample ev422. FIG. 25G shows expression of Col1α1 in sample ev430. FIG. 25H shows expression of TNF-α in sample ev430. FIG. 25I shows IL-6 expression in sample ev430. *=p<0.05.

FIG. 26 shows the Western diet intake of male NASH model mice (mouse/day) (FIG. 26A), Western diet intake of female NASH model mice (mouse/day) (FIG. 26B), sugar intake of male NASH model mice (mouse/day) (FIG. 26C), and sugar intake of female NASH model mice (mouse/day) (FIG. 26D) in mice with no treatment, vehicle treatment, treatment with AZD3355 at 10 and 30 mg/kg, and OCA at 30 mg/kg.

FIG. 27 shows the body weight of male NASH model mice (FIG. 27A) and female NASH model mice (FIG. 27B) in mice with no treatment, vehicle treatment, treatment with AZD3355 at 10 and 30 mg/kg, and OCA at 30 mg/kg. *=p<0.05.

FIG. 28 shows that tumor number (percent/group) of male NASH model mice (FIG. 28A) and female NASH model mice (FIG. 28B) in mice with no treatment, vehicle treatment, treatment with AZD3355 at 10 and 30 mg/kg, and OCA at 30 mg/kg.

FIG. 29 shows the liver weight and the liver/body weight ratio in male NASH model mice with no treatment, vehicle treatment, treatment with AZD3355 at 10 and 30 mg/kg, and OCA at 30 mg/kg. FIG. 29A shows liver weight in untreated male NASH model mice at 12 and 24 weeks. FIG. 29B shows liver weight in male NASH model mice with indicated treatments. FIG. 29C shows liver/body weight ratio in untreated male NASH model mice at 12 and 24 weeks. FIG. 29D shows liver/body weight ratio in male NASH model mice with indicated treatments.

FIG. 30 shows the spleen weight and the spleen weight/liver weight ratio in male NASH model mice with no treatment, vehicle treatment, treatment with AZD3355 at 10 and 30 mg/kg, and OCA at 30 mg/kg. FIG. 30A shows spleen weight in untreated male NASH model mice at 12 and 24 weeks. FIG. 30B shows spleen weight in male NASH model mice with indicated treatments. FIG. 30C shows spleen weight/liver weight ratio in untreated male NASH model mice at 12 and 24 weeks. FIG. 30D shows spleen weight/liver weight ratio in male NASH model mice with indicated treatments.

FIG. 31 shows the liver weight and the liver/body weight ratio in female NASH model mice with no treatment, vehicle treatment, treatment with AZD3355 at 10 and 30 mg/kg, and OCA at 30 mg/kg. FIG. 31A shows liver weight in untreated female NASH model mice at 12 and 24 weeks. FIG. 31B shows liver weight in female NASH model mice with indicated treatments. FIG. 31C shows liver/body weight ratio in untreated female NASH model mice at 12 and 24 weeks. FIG. 31D shows liver/body weight ratio in female NASH model mice with indicated treatments.

FIG. 32 shows the spleen weight and the spleen weight/liver weight ratio in female NASH model mice with no treatment, vehicle treatment, treatment with AZD3355 at 10 and 30 mg/kg, and OCA at 30 mg/kg. FIG. 32A shows spleen weight in untreated female NASH model mice at 12 and 24 weeks. FIG. 32B shows spleen weight in female NASH model mice with indicated treatments. FIG. 32C shows spleen weight/liver weight ratio in untreated female NASH model mice at 12 and 24 weeks. FIG. 32D shows spleen weight/liver weight ratio in female NASH model mice with indicated treatments.

FIG. 33 shows graphs of the level of liver enzymes of NASH model mice at 24 weeks as compared to 12 weeks. FIG. 33A shows alanine aminotransferase (SGPT) in males. FIG. 33B shows aspartate aminotransferase (SGOT) in males. FIG. 33C shows total cholesterol in males. FIG. 33D shows total triglycerides in males. FIG. 33E shows alanine aminotransferase (SGPT) in females. FIG. 33F shows aspartate aminotransferase (SGOT) in females. FIG. 33G shows total cholesterol in females. FIG. 33H shows total triglycerides in females.

FIG. 34 shows graphs of the level of liver enzymes of NASH model mice with vehicle treatment, treatment with AZD3355 at 10 and 30 mg/kg, and OCA at 30 mg/kg for 12 weeks. FIG. 34A shows alanine aminotransferase (SGPT) in males with indicated treatment. FIG. 34B shows aspartate aminotransferase (SGOT) in males with indicated treatment. FIG. 34C shows total cholesterol in males with indicated treatment. FIG. 34D shows total triglycerides in males with indicated treatment. FIG. 34E shows alanine aminotransferase (SGPT) in females with indicated treatment. FIG. 34F shows aspartate aminotransferase (SGOT) in females with indicated treatment. FIG. 34G shows total cholesterol in females with indicated treatment. FIG. 34H shows total triglycerides in females with indicated treatment.

FIG. 34 is a graph of GADPH expression in NASH mice with vehicle treatment, treatment with AZD3355 at 10 and 30 mg/kg, and OCA at 30 mg/kg.

FIG. 36 shows the profibrotic gene expression in male NASH mice at 24 weeks as compared to 12 weeks. FIG. 36A shows Col1α1 expression. FIG. 36B shows αSMA expression. FIG. 36C shows βPDGF-R expression. FIG. 36D shows TGFβ-R1 expression. FIG. 36E shows TIMP1 expression. FIG. 36F shows TIMP2 expression. FIG. 37G shows MMP2 expression.

FIG. 37 shows the profibrotic gene expression in male NASH mice with vehicle treatment, treatment with AZD3355 at 10 and 30 mg/kg, and OCA at 30 mg/kg for 12 weeks.

FIG. 37A shows Col1α1 expression. FIG. 37B shows αSMA expression. FIG. 37C shows βPDGF-R expression. FIG. 37D shows TGFβ-R1 expression. FIG. 37E shows TIMP1 expression. FIG. 37F shows TIMP2 expression. FIG. 37G shows MMP2 expression.

FIG. 38 shows the profibrotic gene expression in female NASH mice at 24 weeks as compared to 12 weeks. FIG. 38A shows Col1α1 expression. FIG. 38B shows αSMA expression. FIG. 38C shows βPDGF-R expression. FIG. 38D shows TGFβ-R1 expression. FIG. 38E shows TIMP1 expression. FIG. 38F shows TIMP2 expression. FIG. 38G shows MMP2 expression.

FIG. 39 shows the profibrotic gene expression in female NASH mice with vehicle treatment, treatment with AZD3355 at 10 and 30 mg/kg, and OCA at 30 mg/kg for 12 weeks. FIG. 39A shows Col1α1 expression. FIG. 39B shows αSMA expression. FIG. 39C shows βPDGF-R expression. FIG. 39D shows TGFβ-R1 expression. FIG. 39E shows TIMP1 expression. FIG. 39F shows TIMP2 expression. FIG. 39G shows MMP2 expression.

FIG. 40 are western blots of the profibrotic protein expression in the whole livers of male NASH mice at 12 weeks and 24 weeks with no treatment. FIG. 40A are male NASH mice at 12 weeks. FIG. 40B are male NASH mice at 24 weeks. FIG. 40C are female NASH mice at 12 weeks. FIG. 40D are female NASH mice at 24 weeks.

FIG. 41 are graphs of the densitometric analysis of western blots of fibrogenic protein expression relative to GADPH. FIG. 41A is a graph of Col1α1 protein expression in male NASH mice. FIG. 41B is a graph of αSMA protein expression in male NASH mice. FIG. 41C is a graph of Col1α1 protein expression in female NASH mice. FIG. 41D is a graph of αSMA protein expression in female NASH mice.

FIG. 42 are representative western blots of the profibrogenic protein expression of Col1α1 and αSMA in the whole livers of male NASH mice treated with vehicle control (0.5% methylcellulose) (FIG. 42A), 10 mg/kg of AZD3355 (FIG. 42B), 30 mg/kg of AZD3355 (FIG. 42C), or 30 mg/kg of OCA (FIG. 42D).

FIG. 43 are graphs of the densitometric analysis of western blots of fibrogenic protein expression relative to GADPH in male NASH mice treated with vehicle control (0.5% methylcellulose), 10 mg/kg of AZD3355, 30 mg/kg of AZD3355, or 30 mg/kg of OCA. FIG. 43A shows Col1α1 protein expression. FIG. 43B shows αSMA protein expression.

FIG. 44 are representative western blots of the profibrogenic protein expression of Col1α1 and αSMA in the whole livers of female NASH mice treated with vehicle control (0.5% methylcellulose) (FIG. 44A), 10 mg/kg of AZD3355 (FIG. 44B), 30 mg/kg of AZD3355 (FIG. 44C), or 30 mg/kg of OCA (FIG. 44D).

FIG. 45 are graphs of the densitometric analysis of western blots of fibrogenic protein expression relative to GADPH in female NASH mice treated with vehicle control (0.5% methylcellulose), 10 mg/kg of AZD3355, 30 mg/kg of AZD3355, or 30 mg/kg of OCA. FIG. 45A shows Col1α1 protein expression. FIG. 45B shows αSMA protein expression.

FIG. 46 are graphs of the morphometric quantification of percent total fibrosis and collagen accumulation across liver sections of NASH mice stained with picrosirius red/fast green. FIG. 46A shows the total fibrosis in male NASH mice at 12 weeks and 24 weeks with no treatment. FIG. 46B shows the total fibrosis in female NASH mice at 12 weeks and 24 weeks with no treatment.

FIG. 47 are graphs of the morphometric quantification of percent total fibrosis and collagen accumulation across liver sections of male NASH mice treated with vehicle control (0.5% methylcellulose), 10 mg/kg of AZD3355, 30 mg/kg of AZD3355, or 30 mg/kg of OCA, stained with picrosirius red/fast green. FIG. 47A shows the total fibrosis in male NASH mice with indicated treatment. FIG. 47B shows the collagen deposition in male NASH mice with indicated treatment.

FIG. 48 are graphs of the morphometric quantification of percent total fibrosis and collagen accumulation across liver sections of female NASH mice treated with vehicle control (0.5% methylcellulose), 10 mg/kg of AZD3355, 30 mg/kg of AZD3355, or 30 mg/kg of OCA, stained with picrosirius red/fast green. FIG. 48A shows the total fibrosis in female NASH mice with indicated treatment. FIG. 48B shows the collagen deposition in female NASH mice with indicated treatment.

FIG. 49 are graphs of NAFLD activity score (NAS) in liver ranging from 0-8 calculated according to Brunt criteria by the sum of scores of steatosis, hepatocyte ballooning and lobular inflammation indicating NASH was reached in week 12 in the NASH model mice and maintained up to 24 weeks. FIG. 49A shows the NAS score for male NASH mice. FIG. 49B shows the NAS score for female NASH mice.

FIG. 50 are graphs of NAFLD activity score (NAS) in liver ranging from 0-8 calculated according to Brunt criteria by the sum of scores of steatosis, hepatocyte ballooning and lobular inflammation in NASH mice treated with vehicle control (0.5% methylcellulose), 10 mg/kg of AZD3355, 30 mg/kg of AZD3355, or 30 mg/kg of OCA. FIG. 50A shows the NAS score for male NASH mice with indicated treatment. FIG. 50B shows the NAS score for female NASH mice with indicated treatment.

FIG. 51 are graphs of showing histopathological scores (Brunt criteria) of portal inflammation used to assess fibrosis stage and steatohepatitis grade in the liver of 12 week and 24 weeks untreated mice. Fibrosis stage is increased in 24 weeks compare to 12 weeks indicate significant liver injury. FIG. 51A shows portal inflammation in male NASH mice. FIG. 51B shows fibrosis in male NASH mice. FIG. 51C shows steatohepatitis grade in male NASH mice. FIG. 51D shows portal inflammation in female NASH mice. FIG. 51E shows fibrosis in female NASH mice. FIG. 51F shows steatohepatitis grade in female NASH mice.

FIG. 52 are graphs of showing histopathological scores (Brunt criteria) of portal inflammation used to assess fibrosis stage and steatohepatitis grade in the liver of NASH mice treated with vehicle control (0.5% methylcellulose), 10 mg/kg of AZD3355, 30 mg/kg of AZD3355, or 30 mg/kg of OCA. FIG. 52A shows portal inflammation in male NASH mice with indicated treatment. FIG. 52B shows fibrosis in male NASH mice with indicated treatment. FIG. 52C shows steatohepatitis grade in male NASH mice with indicated treatment. FIG. 52D shows portal inflammation in female NASH mice with indicated treatment. FIG. 52E shows fibrosis in female NASH mice with indicated treatment. FIG. 52F shows steatohepatitis grade in female NASH mice with indicated treatment.

### DETAILED DESCRIPTION OF THE INVENTION

The current disclosure is based in part upon the discovery that NASH. NAFLD, HCC, and related liver diseases and conditions can be treated and/or prevented with a GABA_{B} agonist, and in particular AZD3355 and pharmaceutically acceptable salts thereof.

In certain embodiments, the present disclosure relates to the treatment and/or prevention of liver fibrosis of any cause, including NASH, fatty liver disease, non-alcoholic fatty liver disease, adiposity, and hepatocellular carcinoma. In certain embodiments, the liver fibrosis is a result of alcohol, infection including viral, bacterial or parasitic, or immune mediated disorders.

In certain embodiments, the present disclosure relates to use of the AZD3355 compound and salts, solvates and physiologically functional derivatives thereof as a novel therapy, and particularly in the treatment of NASH, NAFLD, HCC, liver fibrosis, HCC, and related liver diseases and conditions.

In a further embodiment, the present disclosure is directed to methods of alleviating, modulating, or inhibiting the development or progress of NASH, NAFLD, HCC, and related liver diseases and conditions.

In a further embodiment, the present disclosure provides a method of treatment and/or prevention of a patient suffering from a disorder such as NASH, NAFLD, HCC, and related liver diseases or conditions, which comprises administering to said patient a therapeutically effective amount of a GAB An agonist, such as AZD3355, or a pharmaceutically acceptable salt, solvate, or a physiologically functional derivative thereof.

In a further embodiment, the present disclosure for the use of a GABA_{B} agonist, such as AZD3355, or a pharmaceutically acceptable salt or solvate thereof, or a physiologically functional derivative thereof, in the preparation of a medicament for the treatment of a disorder including NASH, NAFLD, liver fibrosis, hepatocellular carcinoma, and related liver diseases and conditions.

### Definitions

The terms used in this specification generally have their ordinary meanings in the art, within the context of this invention and the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

The term “subject” as used in this application means an animal with an immune system such as avians and mammals. Mammals include canines, felines, rodents, bovine, equines, porcines, ovines, and primates. Avians include, but are not limited to, fowls, songbirds, and raptors. Thus, the invention can be used in veterinary medicine, e.g., to treat companion animals, farm animals, laboratory animals in zoological parks, and animals in the wild. The invention is particularly desirable for human medical applications.

The term “patient” as used in this application means a human subject. In some embodiments of the present invention, the “patient” is suffering with liver condition including but is not limited to fatty liver disease, nonalcoholic fatty liver disease, adiposity, liver fibrosis, cirrhosis, hepatocellular carcinoma, and combinations thereof.

The terms “treat”, “treatment”, and the like refer to a means to slow down, relieve, ameliorate or alleviate at least one of the symptoms of the disease, or reverse the disease after its onset.

The terms “prevent”, “prevention”, and the like refer to acting prior to overt disease onset, to prevent the disease from developing or minimize the extent of the disease or slow its course of development.

The term “in need thereof” would be a subject known or suspected of having or being at risk of a liver disease or condition.

A subject in need of treatment would be one that has already developed the disease or condition. A subject in need of prevention would be one with risk factors of the disease or condition.

The phrase “therapeutically effective amount” is used herein to mean an amount sufficient to cause an improvement in a clinically significant condition in the subject, or delays or minimizes or mitigates one or more symptoms associated with the disease, or results in a desired beneficial change of physiology in the subject.

The term “agent” as used herein means a substance that produces or is capable of producing an effect and would include, but is not limited to, chemicals, pharmaceuticals, biologics, small organic molecules, antibodies, nucleic acids, peptides, and proteins.

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

### Molecular Biology

In accordance with the present invention, there may be numerous tools and techniques within the skill of the art, such as those commonly used in molecular immunology, cellular immunology, pharmacology, and microbiology. See, e.g., Sambrook et al. (2001) Molecular Cloning: A Laboratory Manual. 3rd ed. Cold Spring Harbor Laboratory Press: Cold Spring Harbor, N.Y.; Ausubel et al. eds. (2005) Current Protocols in Molecular Biology. John Wiley and Sons, Inc.: Hoboken, N.J.; Bonifacino et al. eds. (2005) Current Protocols in Cell Biology. John Wiley and Sons, Inc.: Hoboken, N.J.; Coligan et al. eds. (2005) Current Protocols in Immunology, John Wiley and Sons, Inc.: Hoboken, N.J.; Coico et al. eds. (2005) Current Protocols in Microbiology, John Wiley and Sons, Inc.: Hoboken, N.J.; Coligan et al. eds. (2005) Current Protocols in Protein Science, John Wiley and Sons, Inc.: Hoboken, N.J.; and Enna et al. eds. (2005) Current Protocols in Pharmacology, John Wiley and Sons, Inc.: Hoboken, N.J.

### Abbreviations

AZD3355: referred to commercially as lesogaberan;
[(2R)-3-amino-2-fluoropropyl]phosphinic acid, 344413-67-8

Molecular Formula: C₃H₈FNO₂P+

Molecular Weight: 140.073285 g/mol

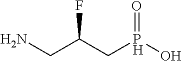

### [(2R)-3-Amino-2-fluoropropyl)phosphinic Acid AZD-3355

NASH: Nonalcoholic steatohepatitis
NAFLD: Nonalcoholic fatty liver disease
HCC: hepatocellular carcinoma
A549: adenocarcinomic human alveolar basal epithelial cells.
MCF7: breast cancer cell line.
LX-2: immortalized human hepatic stellate cells
phHPSC: primary human hepatic stellate cells
ALT: alanine aminotransferase
AST: aspartate aminotransferase

### Nonalcoholic Steatohepatitis (NASH)

### Nonalcoholic Steatohepatitis Symptoms

Most people with NASH have no symptoms. Occasionally, NASH is associated with the symptoms of fatigue, a general feeling of being unwell, and a vague discomfort in their upper right abdomen. Although the cause of NASH is unknown, it is most frequently seen in people with one of more of the following conditions.
Obesity—More than 70 percent of people with NASH are obese. Most obese people with NASH are between 10 and 40 percent heavier than their ideal body weight. Diabetes—Up to 75 percent of people with NASH have type 2 diabetes.
Hyperlipidemia—About 20 to 80 percent of people with NASH have hyperlipidemia (high blood triglyceride levels and/or high blood cholesterol levels).
Insulin resistance—Insulin resistance refers to a state in which the body does not respond adequately to insulin. Insulin resistance often occurs in people with hyperlipidemia who are obese; this group of symptoms is known as the metabolic syndrome and is frequently seen in people with NASH.
Drugs and toxins—Several drugs used to treat medical conditions have been linked to NASH, including amiodarone (brand names: Corderone, Pacerone), tamoxifen (brand names: Nolvadex, Tamone), perhexiline maleate (brand name: Pexhid), steroids (e.g., prednisone, hydrocortisone), and synthetic estrogens. Pesticides that are toxic to cells have also been linked to NASH.

### Nonalcoholic Steatohepatitis Diagnosis

NASH is most often discovered during routine laboratory testing. Additional tests help confirm the presence of NASH and rule out other types of liver disease. Imaging tests (such as ultrasound, CT scan, or magnetic resonance imaging [MRI]) may reveal fat accumulation in the liver but cannot differentiate NASH from other causes of liver disease that have a similar appearance. A liver biopsy may be required to confirm NASH if other causes of liver disease cannot be excluded.

Liver Function Tests

Blood tests to measure the liver function measure levels of substances produced or metabolized by the liver can help to diagnose NASH and differentiate NASH from alcoholic hepatitis. Levels of two liver enzymes (aspartate aminotransferase [AST] and alanine aminotransferase [ALT]) are elevated in about 90 percent of people with NASH. Additional blood tests are useful for ruling out other causes of liver disease and these typically include tests for viral hepatitis (hepatitis A, B, or C).

Liver Biopsy and Fibroscan

Although other tests may suggest a diagnosis of NASH, sometimes a liver biopsy is required for confirmation. A liver biopsy may be needed if other causes of liver disease cannot be ruled out with standard blood and imaging tests. A liver biopsy can also help determine the severity of inflammation, detect liver scarring (fibrosis or, when severe, cirrhosis), and may provide clues about the future course of the condition. The procedure involves collecting a small sample of liver tissue, which is sent to a laboratory for microscopic examination and biochemical testing. Fibroscan is a noninvasive test that uses ultrasound to determine how “stiff” the liver is. This stiffness can then be used to estimate how much scarring there is in the liver and to determine if cirrhosis has developed. Where available, fibroscan is the desirable alternative to liver biopsy for detecting liver scarring.

### Nonalcoholic Steatohepatitis Treatment

There is no cure for NASH. Treatment aims to control the conditions that are associated with NASH such as obesity, diabetes, and hyperlipidemia. Weight reduction can help to reduce levels of liver enzymes, insulin, and can improve quality of life. Weight loss should be gradual (no more than 3.5 lbs or 1.6 kg per week) since rapid weight loss has been associated with worsening of liver disease. Several drugs are available for people with insulin resistance, and these are being studied in patients with NASH.

### Nonalcoholic Steatohepatitis Prognosis

NASH is typically a chronic condition (i.e., it persists for many years). It is difficult to predict the course of NASH in an individual. Few factors have been useful in predicting the course of this condition, although features in the liver biopsy can be helpful.

However, NASH can progress in some people. An initial study that tracked liver damage over time showed that the condition improved in about 3 percent of people, remained stable in 54 percent of people, and worsened in 43 percent of people.

The most serious complication of NASH is cirrhosis, which occurs when the liver becomes severely scarred. It is estimated that between 8 and 26 percent of people with NASH will develop cirrhosis. Older diabetic women may be at increased risk.

People with NASH often have metabolic syndrome (insulin resistance, obesity, and hyperlipidemia). The metabolic syndrome puts people at increased risk for heart disease. It is expected that treatments for NASH (particularly weight loss) will also help treat the other problems that are part of the metabolic syndrome.

### Biological Rationale for Applicability of AZD3355 to Treat NASH and Other Liver Conditions

The GABA_{B} receptor is a member of the G protein-coupled receptor family. It couples negatively to adenylyl cyclase and to voltage-gated calcium channels. It couples positively to inwardly rectifying potassium channels (Bettler et al., 2004). The GABA receptor type B (GABAB or GABA_{B}) agonist baclofen was introduced as a treatment for spasticity in 1966 (Hudgson and Weightman, 1971). As the majority of reflux episodes occur during transient relaxations of the lower esophageal sphincter (LES) (Dodds et al., 1982), inhibition of these relaxations via GABA_{B} agonism has been explored as therapeutic strategy for the management of gastroesophageal reflux disease (GERD). There have been significant efforts to develop a peripherally-restricted GABA_{B} agonist that lacks the central nervous system side effects that are observed with baclofen. AZD3355 ((R)-(3-amino-2-fluoropropyl) phosphinic acid) is a potent and predominately peripherally acting GABA_{B} receptor agonist with a preclinical therapeutic window superior to baclofen.

Evaluating the role of GABA_{B} in liver offers a compelling biological rationale for the novel therapeutic benefit of GABA_{B} agonism in NASH, hepatic fibrosis, and liver carcinogenesis. GABA_{B} receptor agonism attenuates activation of hepatic stellate cells (HSCs), the principle fibrogenic cell in liver (for review see Lee et al., 2015). In vivo, the GABA_{B} agonist baclofen attenuates injury due to carbon tetrachloride, a standard liver injury toxin that induces hepatic fibrosis (Fan et al., 2013). The findings from this study further suggest that GABA_{B} agonism is not only antifibrotic through its direct effects on HSCs, but also may be hepatoprotective by directly reducing liver cell injury. Moreover, GABA_{B} agonism inhibits the growth of hepatocellular carcinoma cells (Wang et al., 2008, Marengo et al., 2015).

The findings set forth herein that these effects are independent of the central activity as well as ancillary (non-GABA_{B}) mediated effects of baclofen is novel.

As described herein, computational chemogenomic drug analysis indicates that AZD3355 can both reduce liver injury associated with NASH, inhibit the production of collagen and other scar constituents, and even attenuate the risk of liver cancer, a growing and life-threatening consequence of NASH.

Hepatocellular carcinoma (HCC) accounts for the majority of primary liver cancers. It has been well established that HCC can occur in the setting of NASH cirrhosis (Ascha et al., 2010). Multiple retrospective studies of HCC in the setting of NASH support the associations of diabetes and obesity with the risk of HCC as well as suggest advanced fibrosis as significant risks. Insulin resistance and its subsequent inflammatory cascade that is associated with the development of NASH seems to play a significant role in the carcinogenesis of HCC. Given the similarities and tight association between NASH and HCC as well as the computational chemogenomic connection of AZD3355 with both NASH and HCC, it was anticipated that AZD3355 is applicable in the treatment of HCC.

The additional in vitro and in vivo results set forth herein show that AZD3355 can be used to treat liver diseases and conditions, including NASH and HCC. In vitro assays using liver cells and human liver slices showed that AZD3355 treatment decreased expression of profibrotic genes with no toxicity. Further evidence using an in vivo NASH mouse model showed treatment with AZD3355 reduced tumor development in the liver, improved liver and spleen weight, improved necro-inflammatory activity including biochemical markers of liver injury (AST and ALT), and significantly reduced expression of all profibrotic genes without any toxic effects on the mice.

### GABA_{B} Receptor Agonist AZD3355

Lesogaberan (AZD3355) was developed by AstraZeneca for the treatment of gastroesophageal reflux disease (GERD) (Bredenoord, 2009). As a GABA_{B} receptor agonist (Alstermark, et al. 2008) it has the same mechanism of action as baclofen but is anticipated to have fewer of the central nervous system side effects that limit the clinical use of baclofen for the treatment of GERD (Lacy et al. 2010). As shown herein, treatment with AZD3355 was not toxic to cells or mice.

The following AZD3355-related patents are incorporated by reference herein: U.S. Pat. Nos. 7,557,234, 8,026,384, 6,664,069, 6,117,908, 7,319,095, 6,841,698, 7,034,176, 7,807,658, and 6,576,626.

### Pharmaceutical Compositions and Administration

While it is possible that, for use in therapy, any of the therapeutic compounds, such as AZD3355, as well as salts, solvates and physiological functional derivatives thereof, may be administered as the raw chemical, it is possible to present the active ingredient as a pharmaceutical composition. Accordingly, the disclosure further provides a pharmaceutical composition, which comprises a therapeutically effective amount of the AZD3355 compound, and salts, solvates and physiological functional derivatives thereof, and one or more pharmaceutically acceptable carriers. The AZD3355 compound, and salts, solvates and physiological functional derivatives thereof, are as described herein.

The phrase “pharmaceutically acceptable” as used herein refers to molecular entities and compositions that are physiologically tolerable and do not typically produce an allergic or similar untoward reaction, such as gastric upset, dizziness and the like, when administered to a human, and approved by a regulatory agency of the Federal or a state government or listed in the U.S. Pharmacopeia or other generally recognized pharmacopeia for use in animals, and more particularly in humans.

The term “carrier” refers to a diluent, adjuvant, excipient, or vehicle with which the therapeutic is administered. Such pharmaceutical carriers can be sterile liquids, such as saline solutions in water and oils, including those of petroleum, animal, vegetable, or synthetic origin, such as peanut oil, soybean oil, mineral oil, sesame oil, and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents.

The carrier(s) must be acceptable in the sense of being compatible with the other ingredients of the formulation and not deleterious to the recipient thereof.

Pharmaceutical compositions of the present disclosure may be adapted for administration by any appropriate route, for example by the oral (including buccal or sublingual), inhaled, nasal, ocular, or parenteral (including intravenous and intramuscular) route. Such compositions may be prepared by any method known in the art of pharmacy, for example by bringing into association the active ingredient with the carrier(s) or excipient(s).

In a further embodiment, the present disclosure provides a pharmaceutical composition adapted for administration by the oral route, for the treatment of diseases and conditions related to NASH, NAFLD, liver fibrosis, hepatocellular carcinoma, and related liver conditions.

Pharmaceutical compositions of the present disclosure which are adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture, as described above, and filling formed gelatin sheaths, Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acacia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups cart be prepared by dissolving the compound in a suitably flavored aqueous solution, while elixirs are prepared through the use of a non-toxic alcoholic vehicle. Suspensions can be formulated by dispersing the compound in a non-toxic, vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

It should be understood that in addition to the ingredients particularly mentioned above, the compositions may include other agents conventional in the art having regard to the type of formulation in question, for example those suitable for oral administration may include flavouring agents.

A therapeutically effective amount of a compound for use in the present methods will depend upon a number of factors including, for example, the age and weight of the animal, the precise condition requiring treatment and its severity, the nature of the formulation, and the route of administration, and will ultimately be at the discretion of the attendant physician or veterinarian. However, an effective amount of the AZD3355 compound for the treatment of diseases or conditions associated with NASH, NAFLD, HCC, and related liver conditions, will generally be in the range of about 5 μg to 100 mg/kg body weight of recipient (mammal) per day and more usually in the range of about 50 μg to 50 mg/kg body weight per day, and more usually in the range of about 1 mg to 100 mg/kg body weight per day, and more usually in the range of about 5 mg to 75 my/kg body weight per day, and more usually in the range of about 20 mg to 60 mg/kg body weight per day. This amount may be given in a single dose per day or more usually in a number (such as two, three, four, five or six) of sub-doses per day such that the total daily dose is the same. An effective amount of a salt or solvate, thereof, may be determined as a proportion of the effective amount of the AZD3355 compound, per se.

Doses can be adjusted to optimize the effects in the subject. For example, the AZD3355 can be administered at a low dose to start and then increased over time to depending upon the subject's response. A subject can be monitored for improvement of their condition prior to changing, i.e., increasing or decreasing, the dosage. A subject can also be monitored for adverse effects prior to changing the dosage, i.e., increasing or decreasing, the dosage.

Pharmaceutical compositions may be presented in unit dose forms containing a predetermined amount of active ingredient per unit dose. Such a unit may contain, for example, 5 μg to 1 g, preferably 1 mg to 700 mg, more preferably 10 mg to 240 mg of an AZD3355 compound, depending on the condition being treated, the route of administration and the age, weight and condition of the patient. Such unit doses may therefore be administered more than once a day. Preferred unit dosage compositions are those containing a daily dose or sub-dose (for administration more than once a day), as herein above recited, or an appropriate fraction thereof, of an active ingredient. Furthermore, such pharmaceutical compositions may be prepared by any of the methods well known in the pharmacy art.

Compounds of the present disclosure, and their salts and solvates, and physiologically functional derivatives thereof, may be employed alone or in combination with other therapeutic agents for the treatment of diseases and conditions related to NASH, NAFLD, liver fibrosis, hepatocellular carcinoma, and related liver conditions. Combination therapies according to the present disclosure thus comprise the administration of at least one AZD3355 compound, or a pharmaceutically acceptable salt or solvate thereof, or a physiologically functional derivative thereof.

The AZD3355 compound and the other pharmaceutically active agent(s) may be administered together or separately and, when administered separately this may occur simultaneously or sequentially in any order. The amounts of the AZD3355 compound and the other pharmaceutically active agent(s) and the relative timings of administration will be selected in order to achieve the desired combined therapeutic effect.

The individual compounds of such combinations may be administered either sequentially or simultaneously in separate or combined pharmaceutical compositions. Preferably, the individual compounds will be administered simultaneously in a combined pharmaceutical composition. Appropriate doses of known therapeutic agents will be readily appreciated by those skilled in the art.

It will be clear to a person skilled in the art that, where appropriate, the therapeutic ingredient(s) may be used in the form of salts, for example as alkali metal or amine salts or as acid addition salts, or prodrugs, or as esters, for example lower alkyl esters, or as solvates, for example hydrates, to optimize the activity and/or stability and/or physical characteristics, such as solubility, of the therapeutic ingredient. It will be clear also that, where appropriate, the therapeutic ingredients may be used in optically pure form.

The compound referred to above may conveniently be presented for use in the form of a pharmaceutical composition and thus a pharmaceutical composition may further comprise a pharmaceutically acceptable diluent or carrier represent a further aspect of the invention.

The individual compounds of such combinations may be administered either sequentially or simultaneously in separate or combined pharmaceutical compositions. Preferably, the individual compounds will be administered simultaneously in a combined pharmaceutical composition. Appropriate doses of known therapeutic agents will be readily appreciated by those skilled in the art.

Compounds may be prepared by methods known in the art of organic synthesis as set forth in part by the following synthesis schemes. In all of the schemes described below, it is well understood that protecting groups for sensitive or reactive groups are employed where necessary in accordance with general principles of chemistry. Protecting groups are manipulated according to standard methods of organic synthesis (T. W. Green and P. G. M. Wuts (1991) Protecting Groups in Organic Synthesis, John Wiley & Sons). These groups are removed at a convenient stage of the compound synthesis using methods that are readily apparent to those skilled in the art. The selection of protecting groups as well as the reaction conditions and order of reaction steps shall be consistent with the preparation of compounds of the present invention, Those skilled in the art will recognize if a stereocenter exists in compounds of the present invention. Accordingly, the present disclosure includes all possible stereoisomers and includes not only mixtures of stereoisomers (such as racemic compounds) but the individual stereoisomers as well. When a compound is desired as a single enantiomer, it may be obtained by stereospecific synthesis or by resolution of the final product or any convenient intermediate. Resolution of the final product, an intermediate, or a starting material may be effected by any suitable method known in the art See, for example, Stereochemistry of Organic Compounds by E. L. Elia S. H. Widen, and L. N. Mander (Wiley-Interscience, 1994).

### Kits

Also within the scope of the present disclosure are kits for practicing the methods described herein. Such kits may include agents that peripherally agonize GABA_{B} including AZD3355 for the prevention and/or treatment of liver diseases and conditions including but not limited to fatty liver disease, adiposity, liver fibrosis, cirrhosis, hepatocellular carcinoma, and combinations thereof.

In some embodiments, the kit can comprise instructions for use in any of the methods described herein. The included instructions can comprise a description of administration of the agents to a subject to achieve the intended activity in a subject. The kit may further comprise a description of selecting a subject suitable for treatment based on identifying whether the subject is in need of the treatment.

The instructions relating to the use of the agents described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### EXAMPLES

This invention will be better understood from the Experimental Details, which follow. However, one skilled in the art will readily appreciate that the specific methods and results discussed are merely illustrative of the invention as described more fully in the claims that follow thereafter.

### Example 1—Identification of Novel Indications for AZD3355

Computational chemogenomic drug analysis was used to identify NASH, NAFLD, HCC and cirrhosis (liver fibrosis) in general (i.e., including that from other causes), as novel indications for compound AZD3355. The method used to identify the therapeutic connection between AZD3355 and these indications was based on a modified “connectivity mapping” approach (Lamb et al., 2006) where the transcriptomic signature of a drug, i.e., the genome-wide pattern of mRNA changes measured in treated vs. untreated cells, is compared through computational means to the mRNA signature of a human disease (disease vs. healthy controls). The transcriptomic signatures for AZD3355 were generated by exposing standard A549 and MCF7 cell lines to two distinct concentrations of AZD3355 and also to vehicle controls. RNA was obtained after seven hours of exposure and quantified using single-end RNA-sequencing.

The chemogenomic profiles of AZD3355 were evaluated systematically across transcriptional profiles representing 310 distinct disease indications and the top disease indications were ranked. These analyses identified NASH and other liver diseases including HCC and cirrhosis as a top indication for AZD3355 above hundreds of other potential indications, across multiple experimental conditions. Further, once the strong match between AZD3355 and NASH was identified, the same analysis for all 1,309 compounds in the Connectivity Map was performed against the NASH signatures within the disease transcriptome library and it was found that the connectivity scores ranked in the top 1% of predictions for NASH when ranked among the 1,309 compounds. Therefore, the connection between AZD3355 and NASH was found by these methods to be globally unique and significant across 310 diseases and 1,309 compounds.

TABLE 1
Summary of significant transcriptomic connections for AZD3355
signatures with NASH and related cirrhotic diseases

| | | AZD3355 dosage | |
|---|---|---|---|
| | | Low | High |
| Cell Line | A549 | Nonalcoholic | Nonalcoholic |
| | | steatohepatitis (sig 1) | steatohepatitis (sig 1) |
| | | Hepatic lipidosis | Hepatic lipidosis |
| | | Hepatic cirrhosis | Hepatic cirrhosis |
| | | Hepatocellular carcinoma | Hepatocellular carcinoma |
| | | Hepatocellular carcinoma | Hepatocellular carcinoma |
| | | (hepatitis C related) | (hepatitis C related) |
| | MCF7 | Nonalcoholic | Nonalcoholic |
| | | steatohepatitis (sig 1) | steatohepatitis (sig 1) |
| | | Hepatic cirrhosis | Nonalcoholic |
| | | | steatohepatitis (sig 2) |
| | | | Hepatocellular carcinoma |
| | | | Hepatic cirrhosis |
| | | | Hepatocellular carcinoma |
| | | | (hepatitis C related) |

Analysis of the treated vs. control chemogenomic profile of AZD3355 revealed complex patterns of molecular activity in exposed cells. For example, functional molecular annotations of the “leading edge” genes underlying the negative connectivity scores between AZD3355 and NASH signatures identified differential regulation of GWAS genes harboring high risk variants associated with LDL cholesterol and visceral adiposity, pathways associated with lipid metabolism, adipogenesis, insulin signalling and autophagy, and multiple connections to cell-type signatures implicating hepatocytes, adipocytes, monocytes and macrophages. These findings suggested that AZD3355 induces molecular activities beyond its canonical mechanism of action, which are taken into consideration by this drug repurposing approach.

Although this repurposing method was based on a “signature” approach, it allowed for deeper investigation of putative molecular mechanisms underlying drug/indications pairs. To elucidate a deeper molecular understanding of the connection between AZD3355 and NASH, a gene co-expression network model from liver biopsy samples from a heterogeneous patient population was constructed, including individuals with NASH, steatosis, healthy-obese and healthy controls (Ahrens et al., 2013). This approach facilitated the detection of gene subnetworks that are specifically perturbed in the context of NASH and allowed for association of clinical factors with those network features. The chemogenomic signature of AZD3355 was projected on to these network models, and several distinct and interesting gene co-expression modules that are dysregulated in NASH were identified that were linked to relevant clinical traits, such as the extent of liver fibrosis, and also perturbed specifically by AZD3355. This network analysis further informed and strengthened support for repurposing AZD3355 for NASH, and also identified specific gene modules that may underpin the molecular engagement of key networks in NASH by AZD3355.

### Example 2—Use of AZD3355 for Treatment and Prevention of a Liver Condition or Disease Including NASH as Shown by Cell Culture Assays

### Materials and Methods

Cell culture assays were performed in immortalized human hepatic stellate (LX-2) cells and primary human hepatic stellated cells (phHSCs).

Cells were treated with either a vehicle control of saline water, various concentrations of AZD3355 ranging from 1 nM to 300 nM or 7.5 μM of sorafenib as a positive control.

At various indicated time points, the cells were assessed for cytotoxicity, cellular proliferation, rates of apoptosis, expression of fibrogenic genes and proteins

AZD3355 and Sorafenib Small Molecules:

AZD3355 compound was provided by AstraZeneca. The formula weight and total weight of supplied drug was determined by AstraZeneca. The AZD3355 compound was dissolved in normal saline (0.9% sodium chloride) at 2 mM concentration stock solution followed by a series of working concentration of 1, 3, 10, 30, 100 and 300 nM in DMEM cell culture medium supplemented with 0.1% BSA without antibiotic. Both stock and working solution were made fresh before conducting each experiment. For positive control the cells were treated in parallel with, a kinase inhibitor, sorafenib (LC laboratories, MA Catalog #5-8502, Lot #121952) at 7.5 μM concentration dissolved in DMSO.

Human Hepatic Stellate Cells:

LX-2 cells: Immortalized human hepatic stellate cell line was cultured in Dulbecco's Modified Eagle Medium (DMEM) (ThermoScientific, IL, Cat #11965-092) supplemented with 10% fetal bovine serum 1% and penicillin/streptomycin at 37° C. in 5% CO₂ incubator.

Primary human hepatic stellate cells (phHSCs): The experimental protocols were approved and certified by the Mount Sinai Institutional Review Board. phHSCs were prepared from discarded remnants of surgically resected human livers that lacked patient identifiers. The resected liver pieces were two stepped perfused with Liver Perfusion Medium (ThermoScientific, Cat #17701) followed by 0.05% collagenase (Roche, Ref #11459643001)+0.02% pronase (Roche, Ref #11459643001) in hepatocyte wash medium (ThermoScientific, Cat #17704-024) in presence of DNase. After perfusion the liver tissues were mechanically disrupted and digested in same Collagenase-Pronase-DNase buffer solution at 37° C. for 40 minutes. Enzymatically digested liver cell suspension was filtered through 70 μm cell strainer. HSCs were purified from cellular suspension with double density gradient (52% and 35%) of Percoll (GE Healthcare, Cat #17-0891-01) by 2400 rpm at 4° C. for 30 minutes. The HSCs were collected from upper layer of Percoll gradient, washed in DMEM, cultured and passaged in DMEM supplemented with 10% fetal bovine serum and penicillin/streptomycin at 37° C. in 5% CO₂ incubator.

Overview of Experimental Design:

At the beginning of each experiment, stellate cells (LX-2 cells or phHSCs) were serum-starved overnight in DMEM supplied with 0.1% BSA (without antibiotic) to synchronize metabolic activities of the cells. The cells were then exposed to different working concentration of either AZD3355 or sorafenib for 24, 48 and 72 hours.

Cell Cytotoxicity Assay:

5,000 LX-2 cells or 10,000 phHSCs were plated per well in 96 well plates. Cells were serum starved overnight in DMEM supplemented with 0.1% BSA (without antibiotic). Cells were then incubated with different concentrations of AZD3355 for indicated durations and MTS assays were accomplished using CellTiter 96 AQueous One Solution Cell Proliferation Assay kit (Promega, WI) according to manufacturer's protocol.

Cell Proliferation Assay:

5,000 LX-2 cells or 10,000 phHSCs were plated per well in 96 well plates. After overnight serum starvation in DMEM supplemented with 0.1% BSA (without antibiotic) the cells were exposed to AZD3355 at indicated concentration. At 48 and 72 hours of drug exposure the cells were labeled with BrdU for either 2 hours (LX-2 cells) or 16 hours (phHSCs) at 37° C. Cell Proliferation ELISA, BrdU colorimetric kit (Roche, N.Y.) was used following the manufacturer's instructions.

Cell Apoptosis Assay:

5,000 LX-2 cells or 10,000 phHSCs were plated per well in 96-well clear bottom black plates. After overnight serum starvation in DMEM supplemented with 0.1% BSA (without antibiotic) the cells were exposed to AZD3355 at indicated concentration. For positive apoptotic control the cells were treated with 3% DMSO. After 72 hours of drug exposure the fluorescence signal of Caspase-3 and -7 activities were measured in Synergy HT (BioTek Instrument Inc., VT) spectrofluorometer by using Apo-ONE Homogeneous Caspase-3/7 Assay kit (Promega, WI) according to manufacturer's protocol.

Fibrogenic gene expression in hepatic stellate cells by RT-qPCR:

The following fibrogenic gene expressions were quantified by RT-qPCR:

1. Collagen1α1 (Col1α1);

2. Alpha Smooth Muscle Actin (αSMA);

3. Beta PDGF receptor (β-PDGFR);

4. Transforming growth factor-β receptor1 (TGFβ-R1);

5. Tissue inhibitor of metalloproteinase-1 (TIMP1);

6. Tissue inhibitor of metalloproteinase-2 (TIMP2); and

7. Matrix Metalloproteinase 2 (MMP2).

The kinase inhibitor sorafenib (7.5 μM concentration) was used as positive control and run in parallel. 150,000 LX-2 cells or 200,000 phHSCs per well were plated on 6-well plate dish. Cells were starved overnight in DMEM supplemented with 0.1% BSA (without antibiotic). Cells were then incubated with either AZD3355 or sorafenib at the indicated concentration and duration. Cells were harvested and total RNA was extracted using RNeasy Mini Kit (Qiagen, CA). 0.5 μg of total RNA was used for reverse transcription with ‘RNA to cDNA EcoDry Premix (Double Primed) Kit’ (Clontech, CA). Expression of fibrogenic genes were measured by qPCR using custom designed primers (Sigma-Aldrich, MO) and iQ SYBR Green Supermix (Bio-Rad, CA) on a LightCycler 480 II (Roche Diagnostics Corporation, IN) instrument. Glyceraldehyde-3-phosphate dehydrogenase (GAPDH), ribosomal polymerase II (RPII), α-tubulin and β-actin genes were tested to determine the best fit for housekeeping gene of the study. Of those four housekeeping genes GAPDH expression level (Ct value) was constant across AZD3355 treatment groups and selected as housekeeping gene in LX-2 cells as well as phHSCs. Fibrogenic gene expression were normalized to GAPDH.

Fibrogenic Protein Expression in Hepatic Stellate Cells by Western Blot and Densitometric Analysis from Cell Lysate:

Western blot and RT-qPCR experiments were run in parallel. 150,000 LX-2 cells or 200,000 phHSCs per well were plated on a 6-well plate dish. Cells were starved overnight in DMEM supplemented with 0.1% BSA (without antibiotic). Cells were then incubated with either AZD3355 or sorafenib at the indicated concentration and duration. Cells were harvested and lysed using RIPA buffer (50 mM Tris-HCl pH8.0, 150 mM NaCl, 1% IGEPAL, 0.5% sodium deoxycholate and 0.1% SDS) along with Pierce Protease Inhibitor Mini Tablets, EDTA-Free (ThermoScientific, IL). Total protein was measured by Bradford colorimetric assay using Protein Assay Dye Reagent Concentrate (Bio-Rad, CA). 10 rig of proteins were loading in NuPAGE 4-12% Bis-Tris gel (ThermoScientific, IL). After transferring the protein bands to PVDF membrane the bands were blocked with 5% non-fat milk in 1×PBS. The primary antibodies were used for probed the respective protein bands are rabbit anti-Collagen1 (Bioss, MA), rabbit anti-MMP2 (abcam, MA), rabbit anti-αSMA (abcam, MA) and mouse anti-GAPDH (Millipore, CA). After hybridized with HRP conjugated secondary antibody (either Goat anti-rabbit HRP (Jackson ImmunoResearch Laboratories, PA) or anti-mouse IgG-HRP (Cell Signaling Technology, MA) the membrane was treated with Immobilon Western Chemiluminescent HRP substrate (Millipore, MA) and the signals were captured with Amersham Imager 6000 (GE Healthcare, PA). 210 kD of Col1α1, 72 kD of MMP2 and 42 kD of α-SMA bands were recognized by respective antibody. 37 kD band of GAPDH was probed as loading control. For densitometric measurement of the protein bands, images were exported and analyzed using ImageJ 1.50f software and bands were normalized to the loading control, GAPDH.

Secreted Col1α1 Protein Measurement by ELISA:

Cell culture media from western blot assay protocol were collected for assessment of secreted collagen1α1 in the media. After collection the media were centrifuged at 2,000×g for 10 minutes to remove the cell debris. The sample was diluted at the ratio of 1:1000 into sample diluent buffer and measured secreted Collagen1α1 by using Human Pro-Collagen I alpha 1 SimpleStep ELISA kit (abcam, MA) according to manufacturer's protocol.

αSMA Protein Expression in Hepatic Stellate Cells by Immunocytochemistry:

Protein expression of αSMA in LX-2 cells and phHSCs in presence of AZD3355 small molecule were determined by immune-staining DAB technique. The kinase inhibitor sorafenib (7.5 μM concentration) small molecule was used as positive control and run in parallel. 100,000 LX-2 cells or 80,000 phHSCs were seeded on glass coverslip. Cells were starved overnight in DMEM supplemented with 0.1% BSA (without antibiotic). Cells were then incubated with either AZD3355 or Sorafenib small molecule at the indicated concentration and duration. The cells were washed thoroughly with 1×PBS and fixed in 4% Paraformaldehyde, permeabilized with 0.5% Tween-20 in 1×PBS and blocked in Dako Peroxidase Block (0.03% H2O2, sodium azide; Agilent Technologies, CA). To avoid non-specific antibody binding the cells were re-block with Dako Protein Block Serum-Free reagent (Agilent Technologies, CA). The cells were immunostained with rabbit anti-αSMA (abcam, MA) primary antibodies for overnight. A set of no primary antibody control cell was run in parallel as background control. The secondary antibody used for this study was Dako Labelled Polymer-HRP Anti Rabbit (Agilent Technologies, CA) and incubated for 1 hour. The cells were then treated with Dako DAB-Chromogen (Agilent Technologies, CA). Nuclear counter staining was performed with hematoxylin (Sigma-Aldrich, MO). Antibody signals was captured with Axiocam 503 mono camera (Zeiss, N.Y.) using 10× objective in an AxioImager.Z2 upright microscope (Zeiss, N.Y.). Image acquisitions were analyzed by Zen2 software (Zeiss, N.Y.).

Experimental Data Analysis:

Each experiment was repeated at least three times. The data analysis was accomplished by using different scientific and statistical software (GraphPad Prism, Excel, etc.). Standard error (±SE) was calculated according to student t-test. Unless otherwise specified, p values smaller than 0.05 were considered statistically significant.

### Results

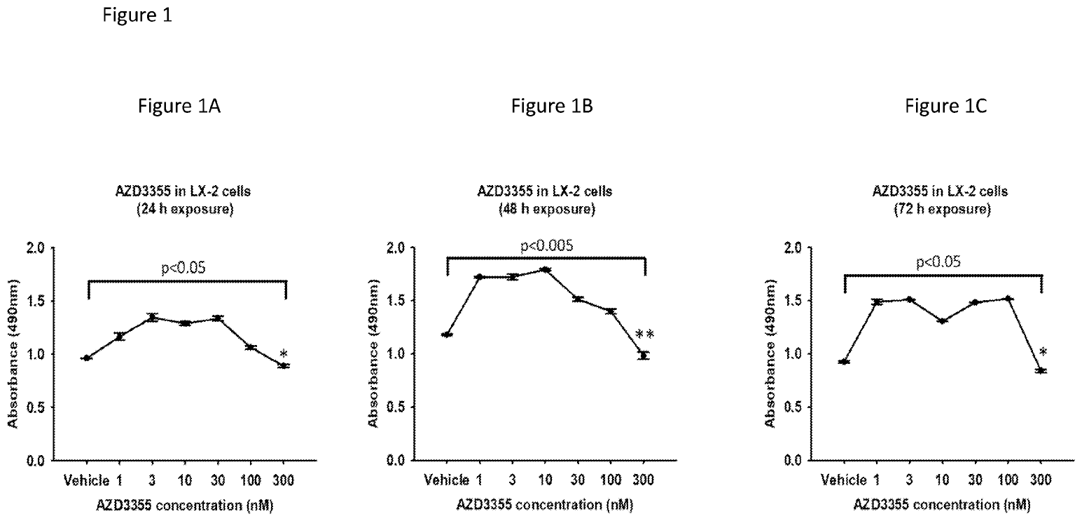

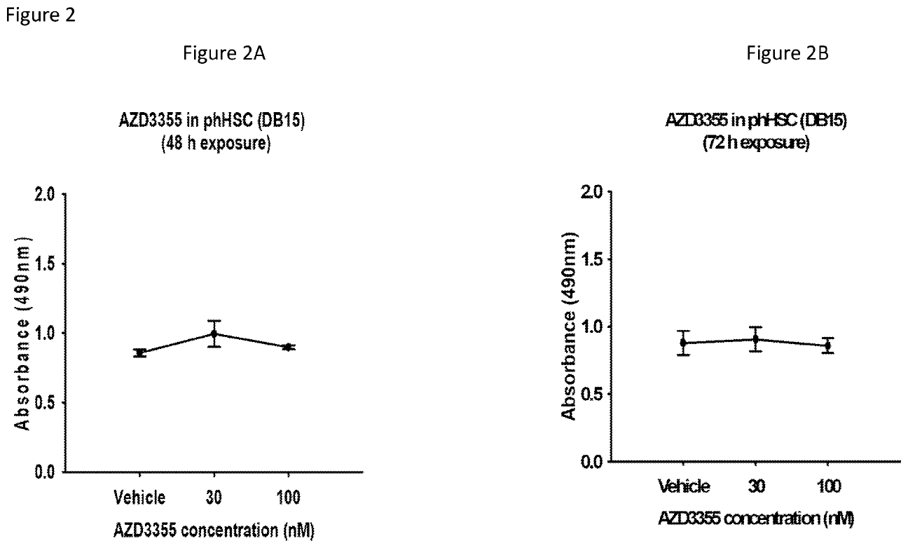

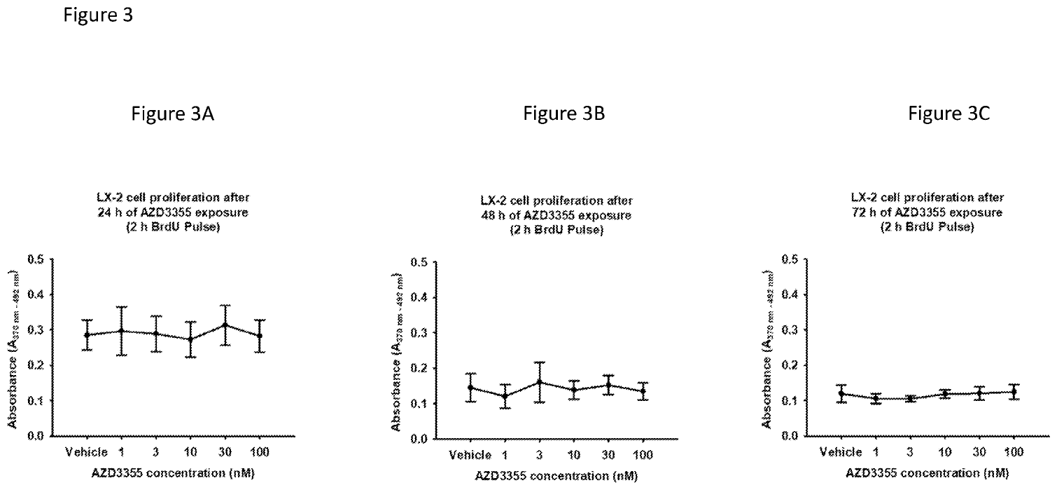

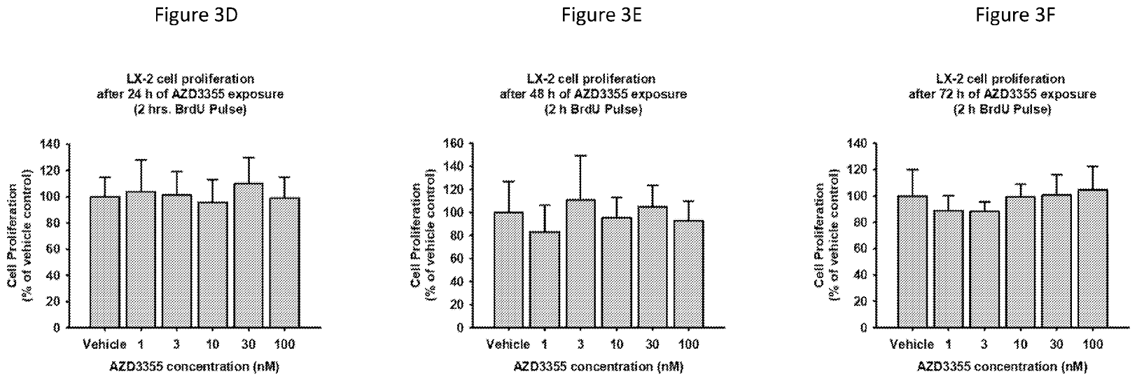

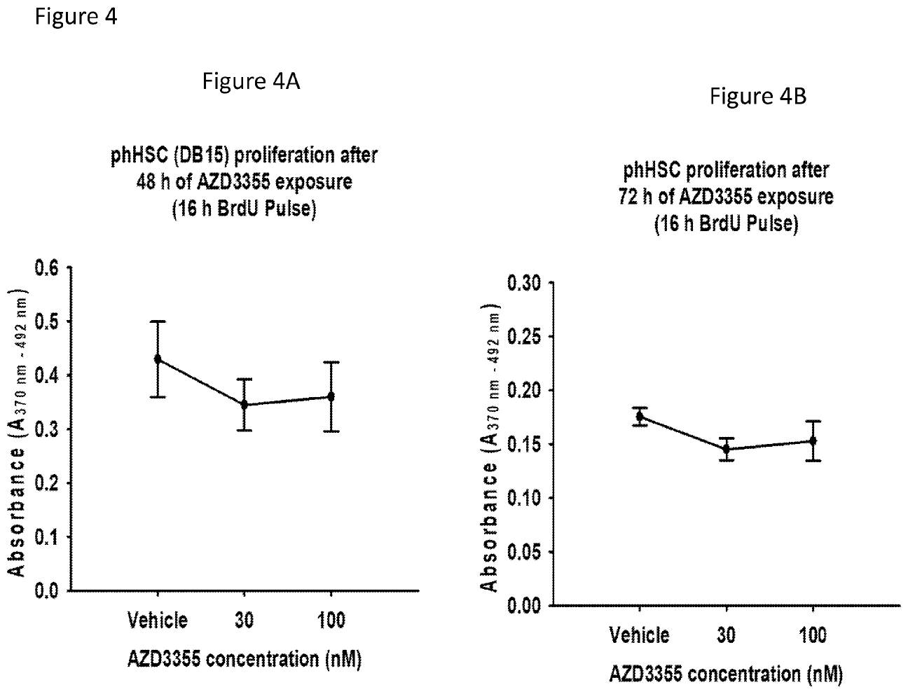

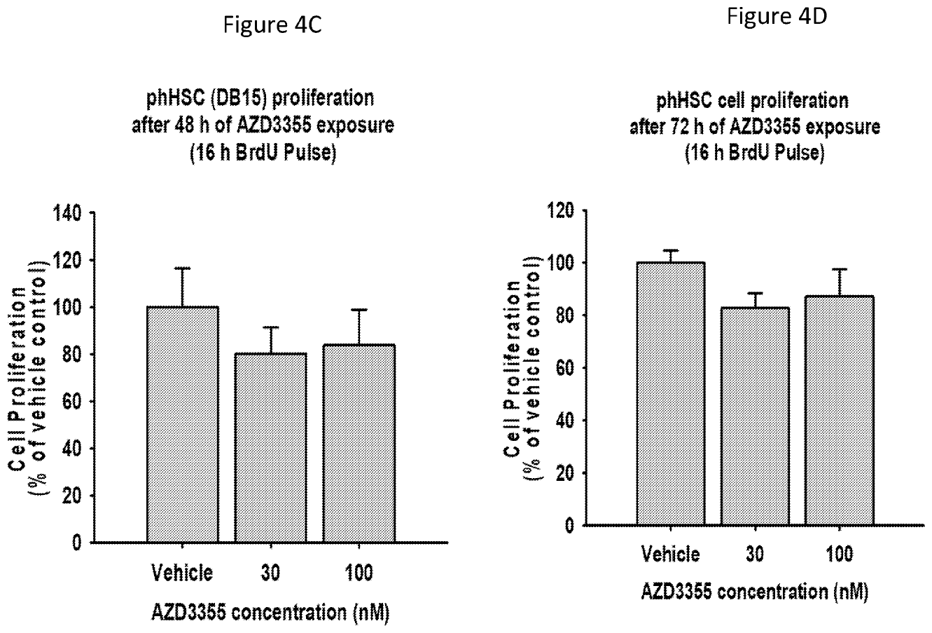

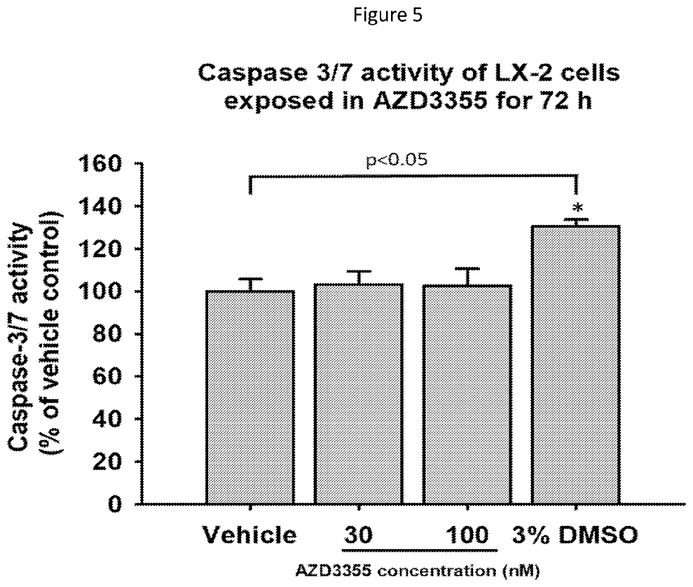

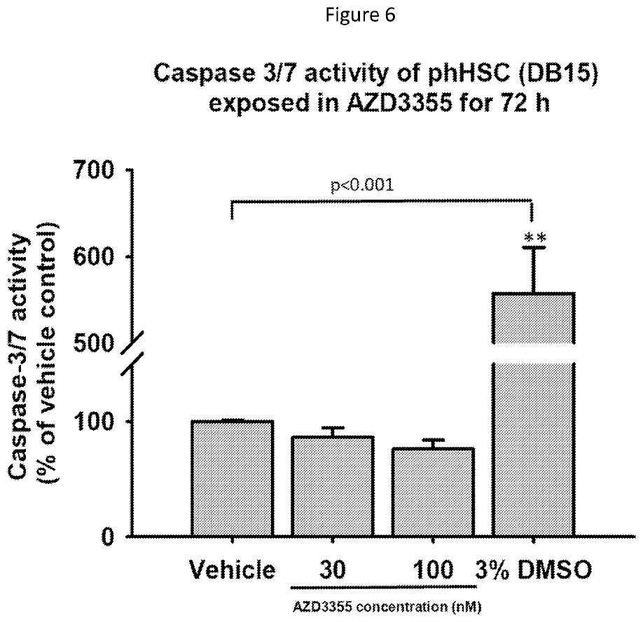

Both LX-2 cells and phHSCs were treated with AZD3355. The former were treated with from 0-300 nM for either 24, 48 or 72 hours. The latter were treated with 30 nM or 100 nM for 48 or 72 hours. There was no significant cell cytotoxicity observed except for in LX-2 cells at 300 nM. See FIGS. 1 and 2. There were also no significant changes in cellular proliferation in either the treated LX-2 cells or the phHSCs. See FIGS. 3 and 4. Cells were also assessed for apoptotic effect of the AZD3355 by measuring caspase-3/7 activity. There was no apoptotic effect of AZD3355 in either the LX-2 cells or phHSCs for any dosage of AZD3355. See FIGS. 5 and 6. These results showed that the drug was not toxic to the cells, i.e., did not harm the cells, cause death of the cells, or inhibit growth of the cells.

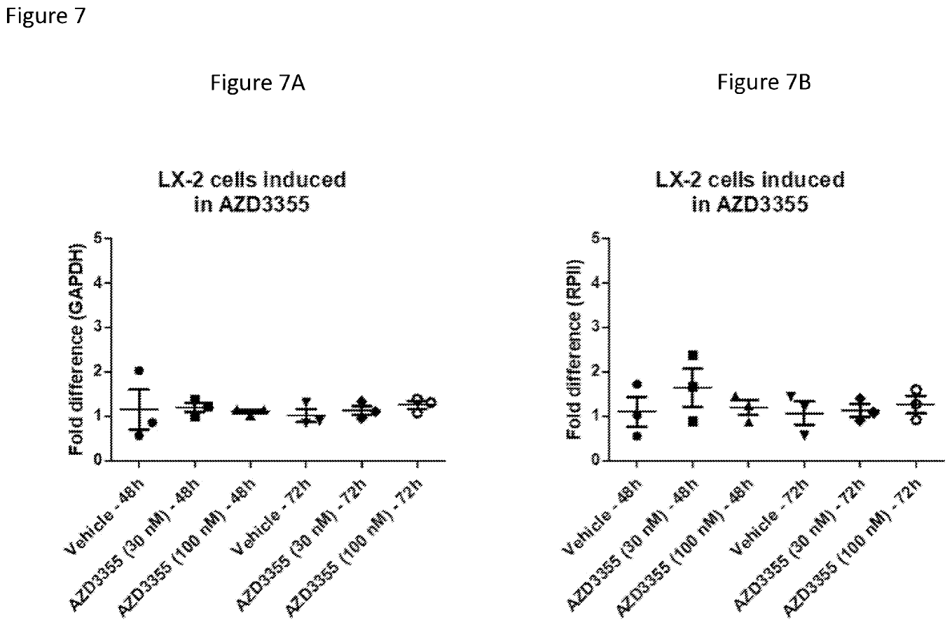

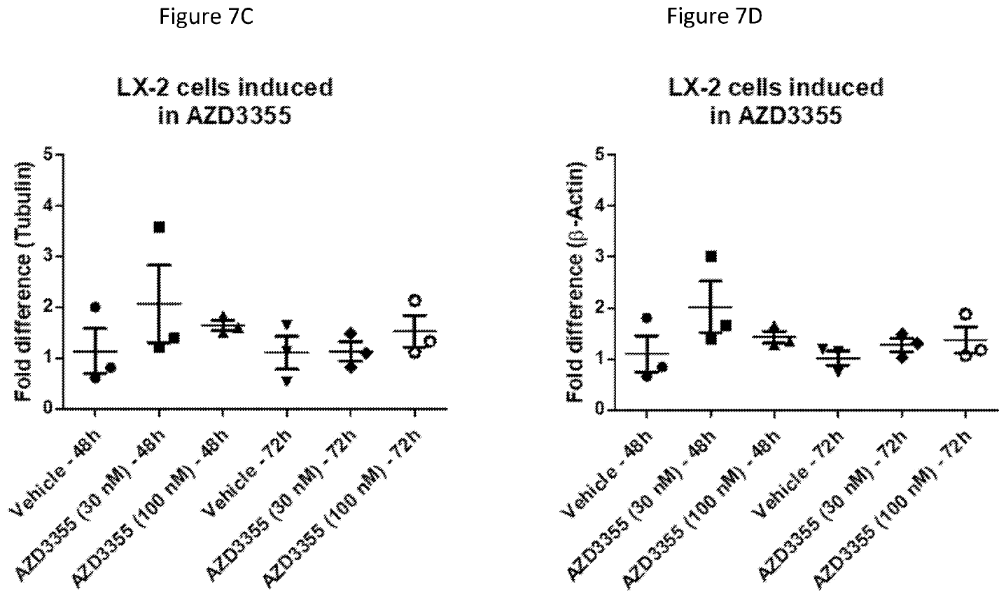

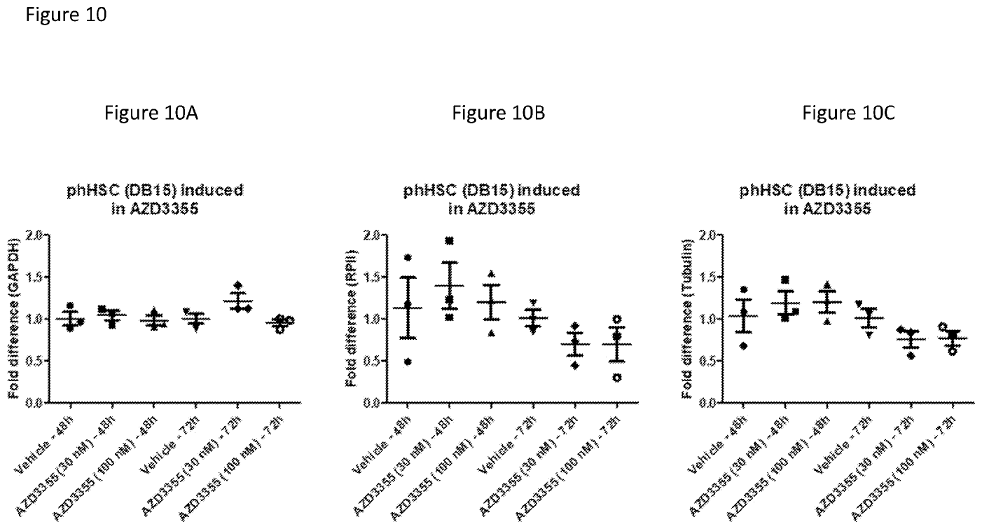

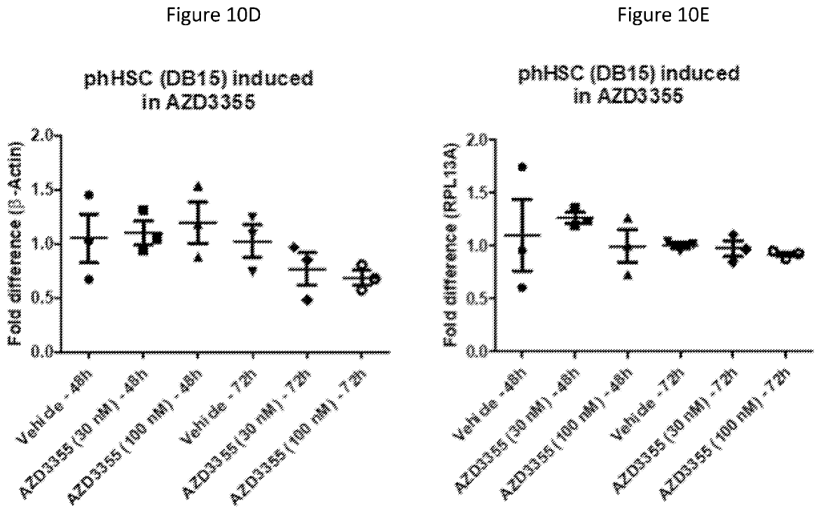

Gene expression was also assessed in both LX-2 and phHSCs treated with AZD3355 using qPCR. Genes included GADPH, RPII, and tubulin. The expression level of GADPH did not change in the either of the cells after treatment with AZD3355. See FIGS. 7 and 10. These results further showed that the drug was not toxic to the cells.

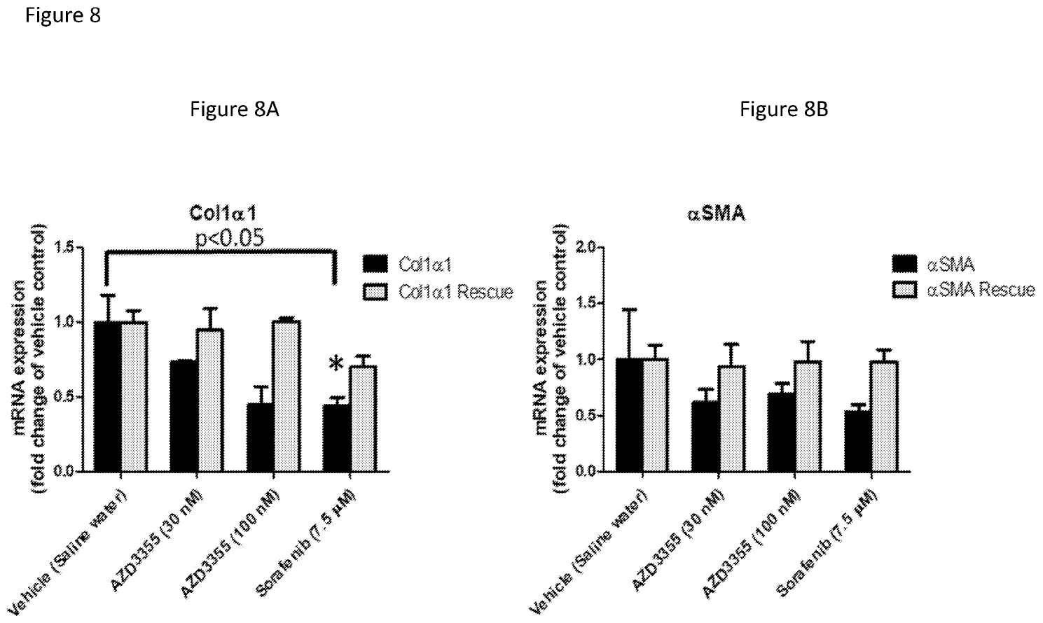

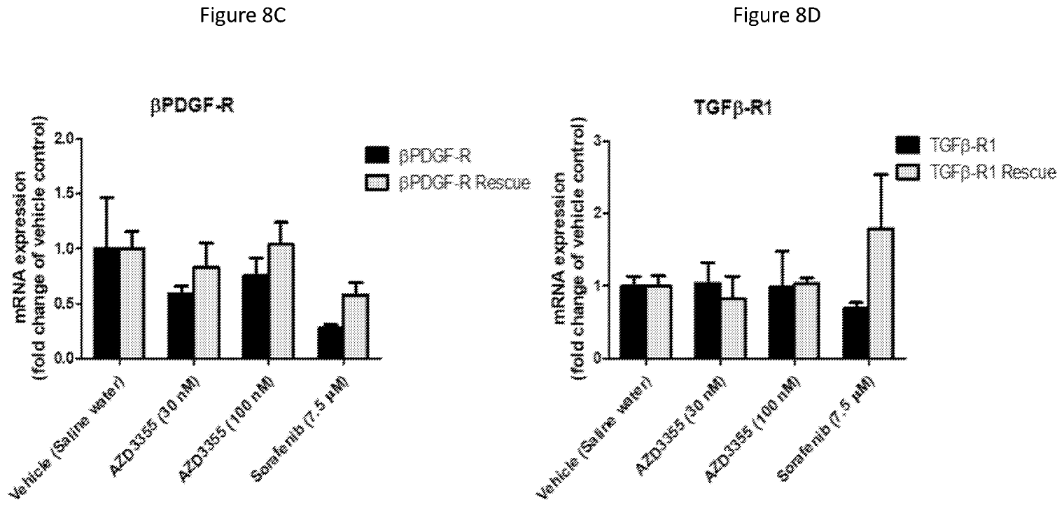

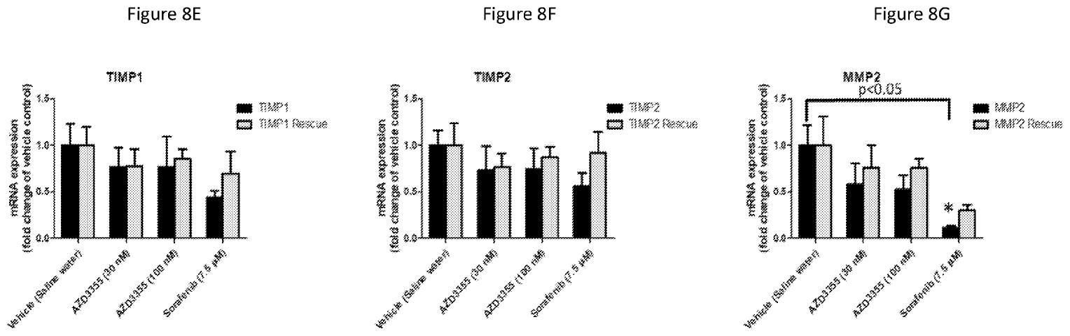

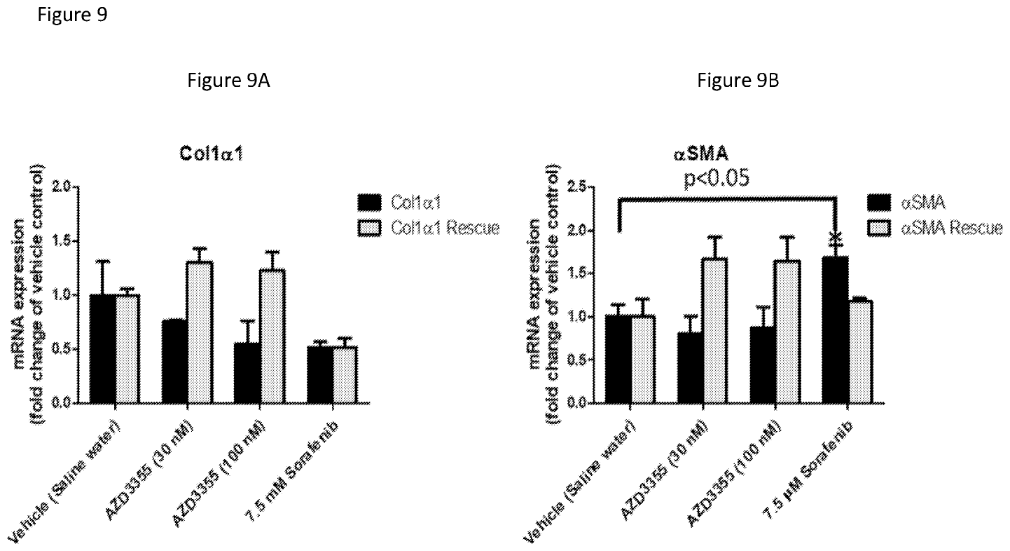

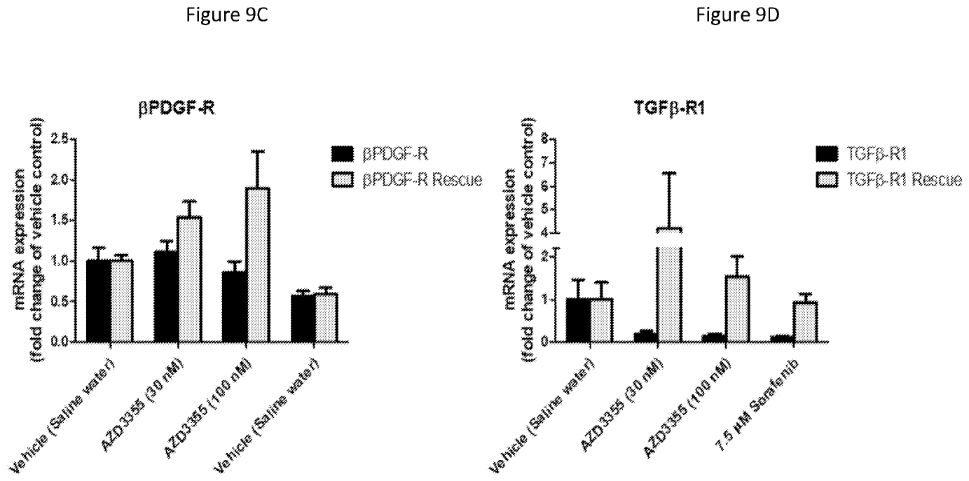

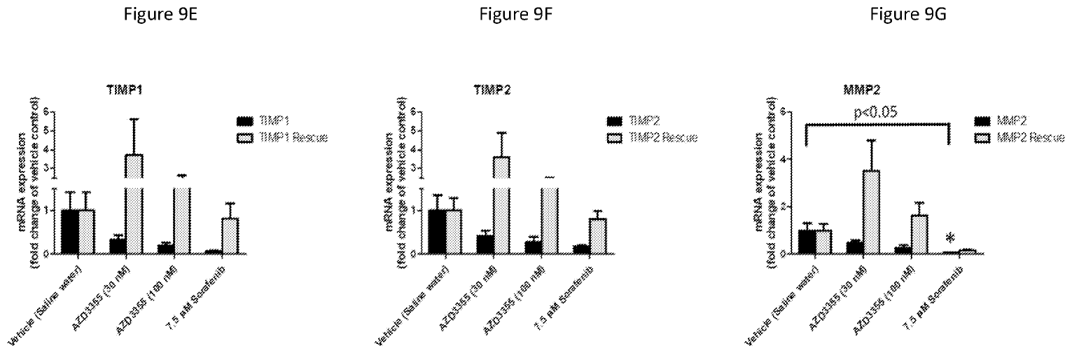

Gene expression of pro-fibrogenic genes was also assessed in both cell lines, treated with vehicle, AZD3355, or sorafenib. These genes including Col1α1, αSMA, βPDGF-R, TGFβ-R1, TIMP1, TIMP2, and MMP2 were downregulated by treatment with AZD3355 or sorafenib at 48 and 72 hours in LX-2 cells. In particular, in FIGS. 8 and 9, the black bars in the graphs show down regulation. When the cells were then maintained in drug free media for an additional 48 or 72 hours, the fibrogenic genes were rescued. See FIGS. 8 and 9, in particular, gray bars in the graphs show the rescue of the gene expression. See also Table 2.

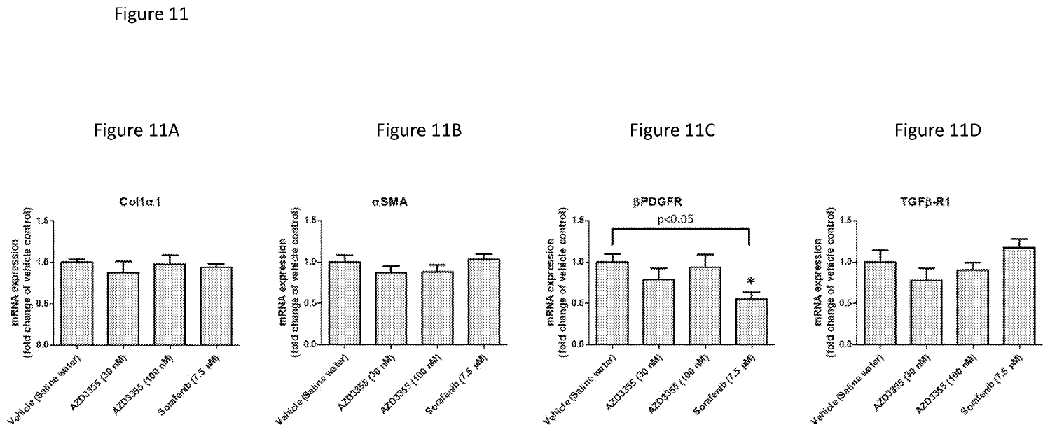

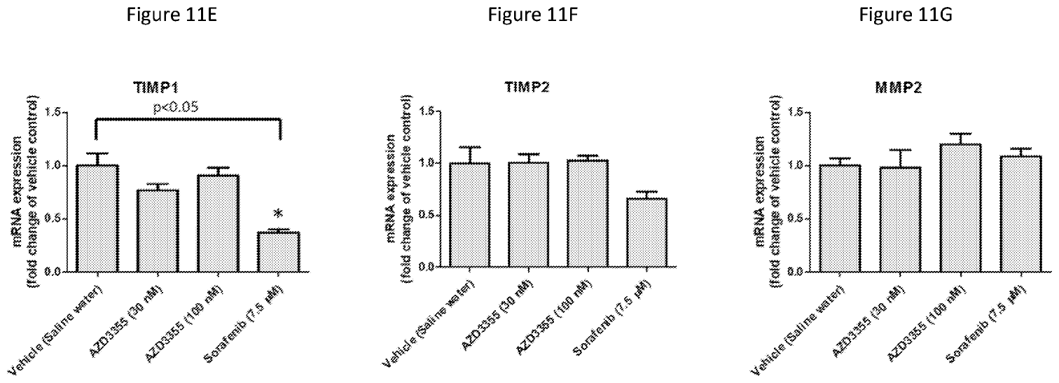

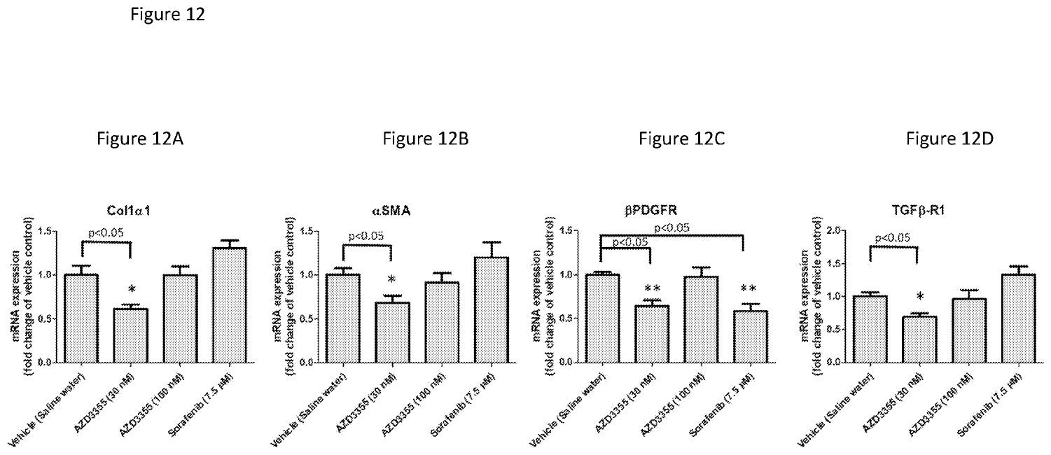

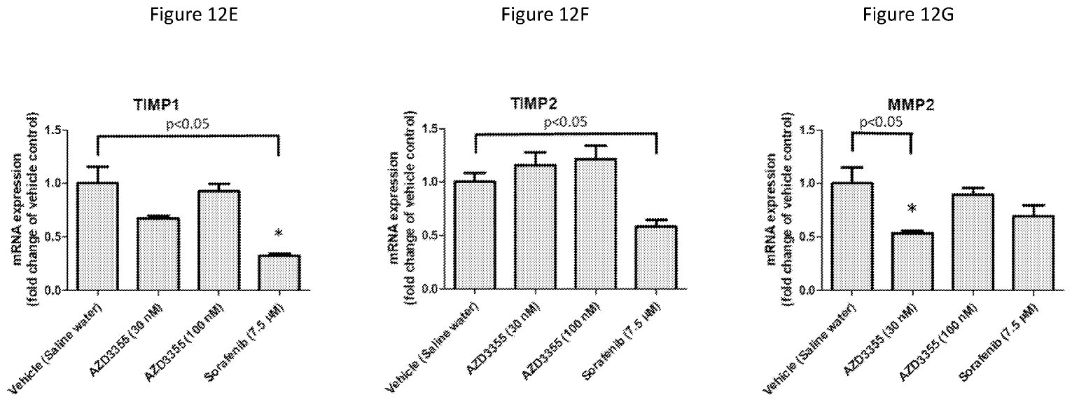

Similar results were obtained with the treatment of the phHSCs with AZD3355 or sorafenib. At 48 hours of treatment, βPDGF-R, TGFβ-R1, and TIMP1 were downregulated with 30 nM of AZD3355. See FIG. 11. At 72 hours of treatment, all of the pro-fibrogenic genes except for TIMP1 and TIMP2 were significantly down-regulated. See FIG. 12. See also Table 3.

These findings showed that the treatment with AZD3355 decreased the expression of genes known to cause fibrogenic conditions in the liver.

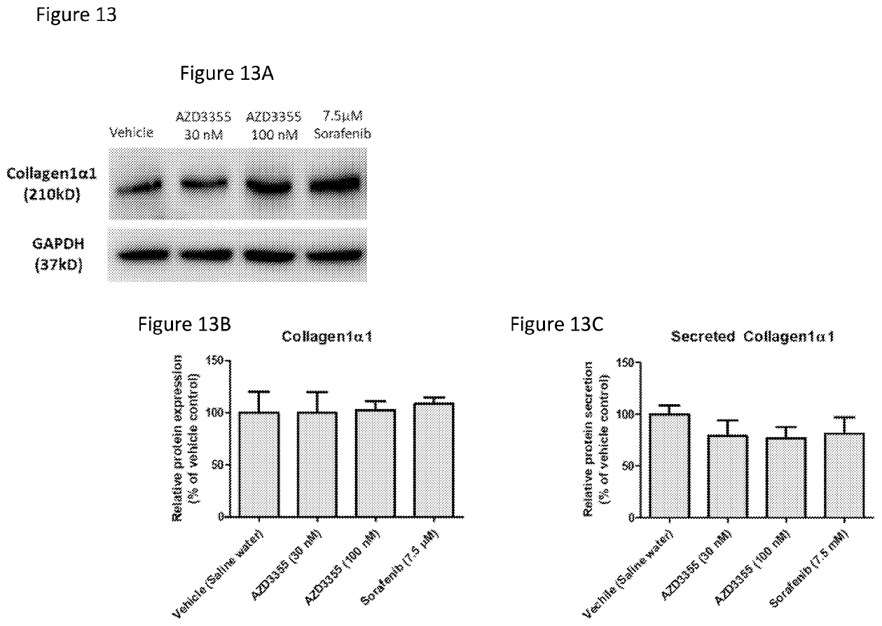

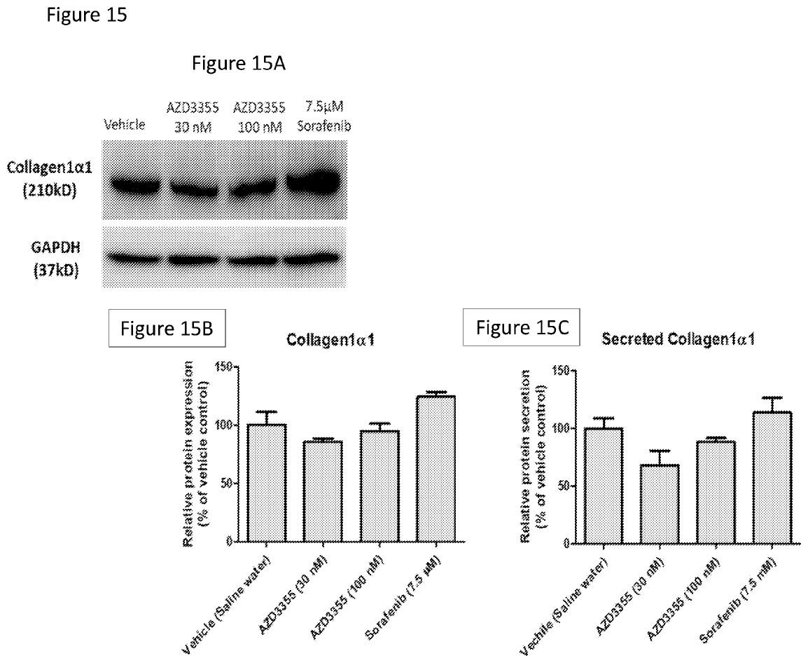

Protein expression of various genes was also assessed in three separate Western blot experiments. Expression of Col1α1, αSMA, and MMP were measured. There was no change in protein expression of Col1α1 in LX2 cells treated for 48 hours or 72 hours with either 30 nM or 100 nM of AZD3355 or sorafenib. See FIGS. 13A and 13B, FIGS. 15A and 15B. However there was a reduction in secreted Col1α1 in culture media as measured by ELISA in the treated cells. See FIGS. 13C and 15C.

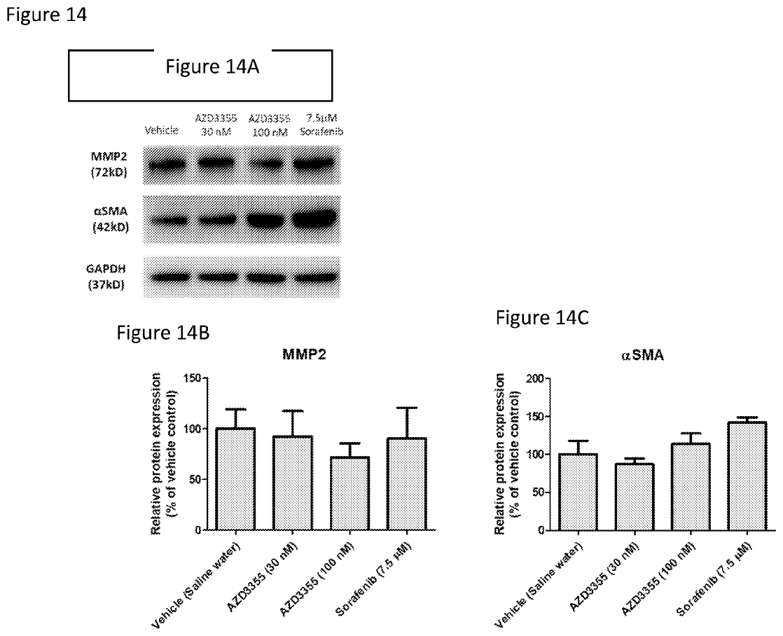

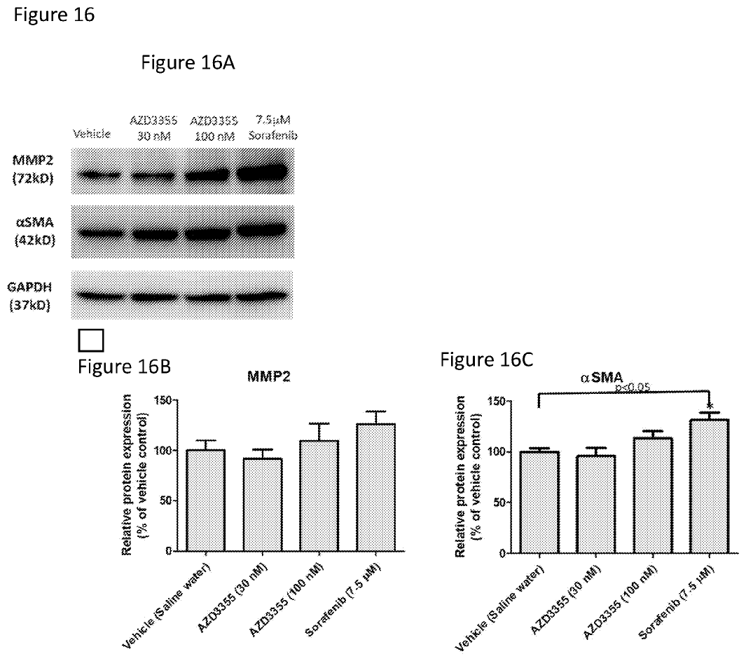

αSMA and MMP protein expression was measured at 48 hours and 72 hours of treatment with 30 nM or 100 nM of AZD3355 or sorafenib. See FIGS. 14 and 16. See also Table 4.

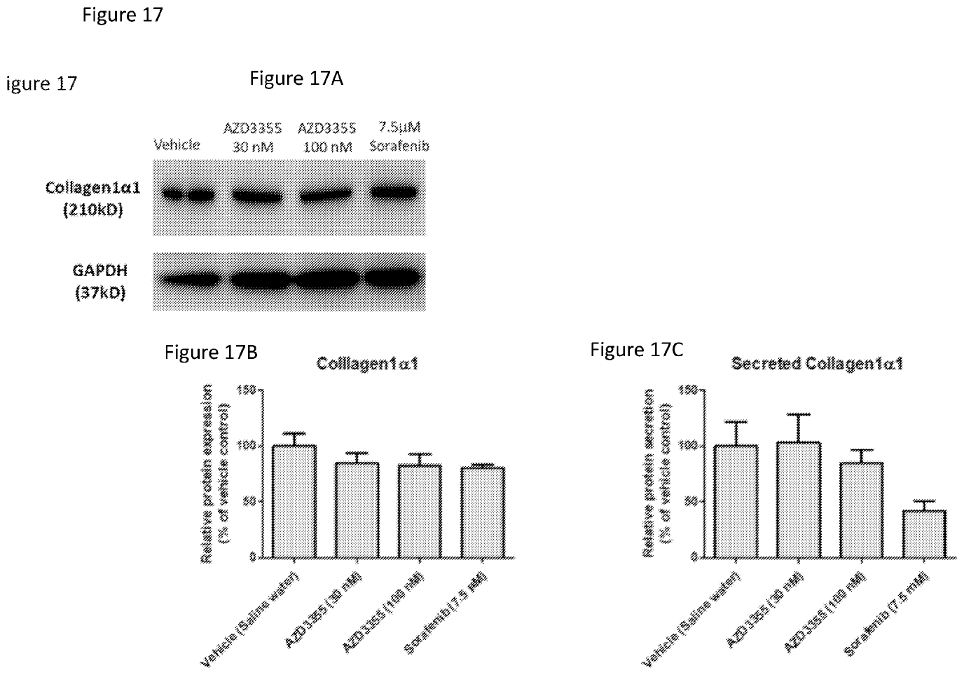

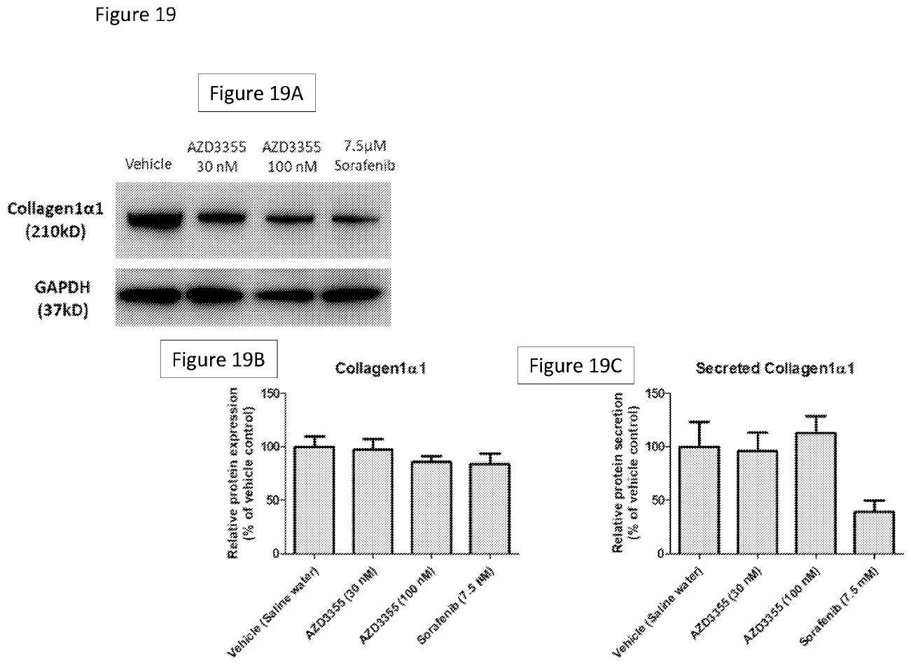

Similar results were obtained for the expression of Col1α1, αSMA, and MMP proteins in phHSCs. There was no change in protein expression of Col1α1 in phHSCs treated for 48 or 72 hours with AZD3355 or sorafenib. FIGS. 17A, 17B, 19A and 19B. However there was a reduction in secreted Col1α1 in culture media as measured by ELISA in cells treated with 100 nM of AZD3355 for 48 hours. See FIG. 17C.

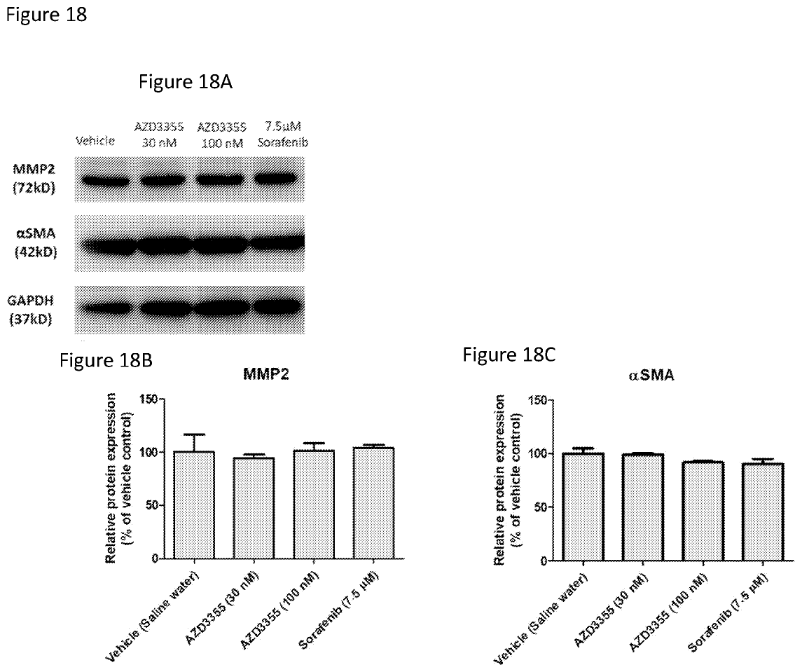

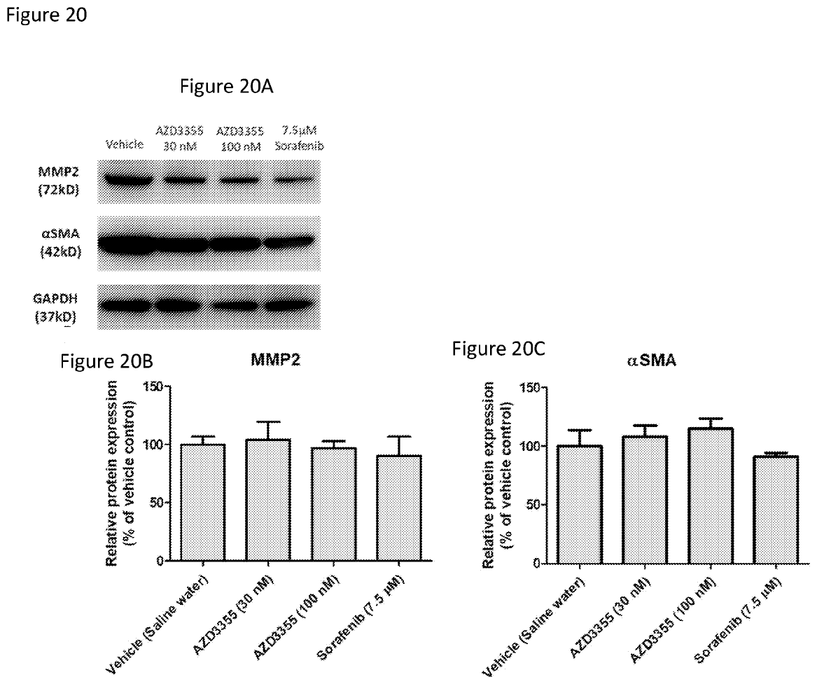

αSMA and MMP protein expression was measured at 48 hours and 72 hours of treatment with 30 nM or 100 nM of AZD3355 or sorafenib. See FIGS. 18 and 20. See also Table 5.

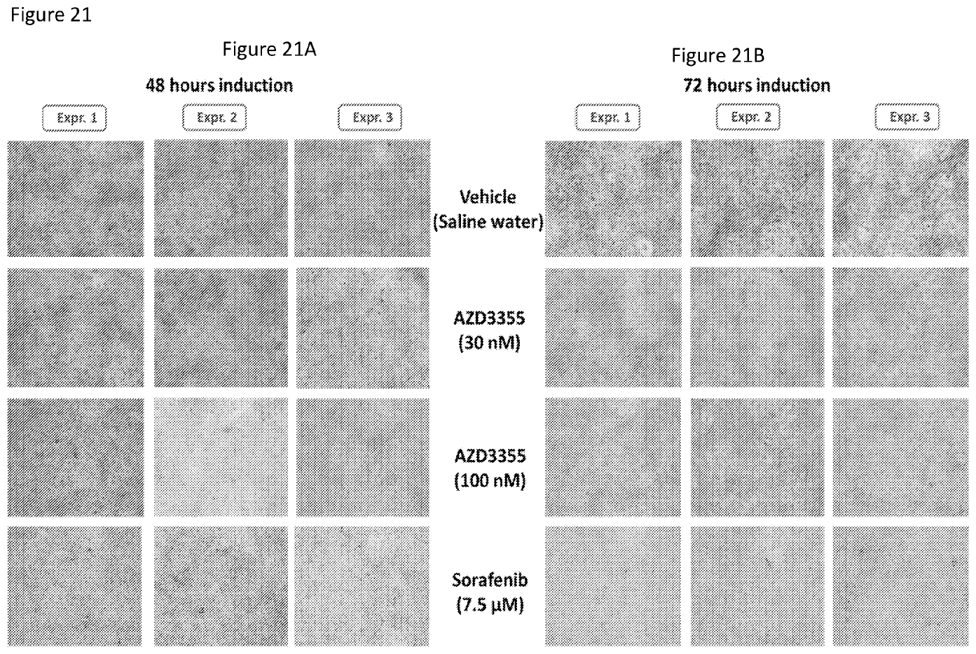

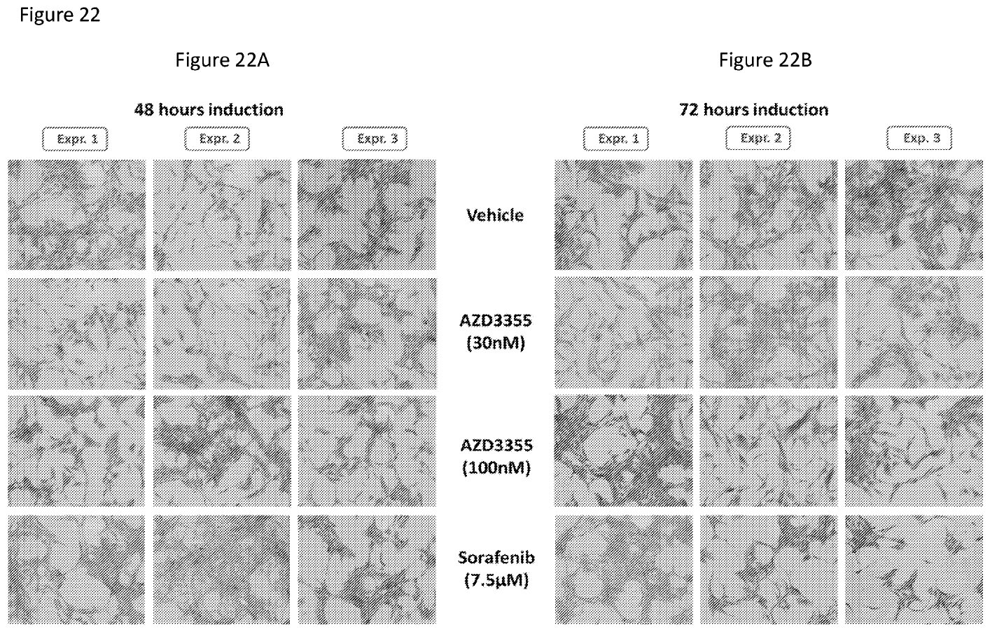

Immunocytochemistry analysis of αSMA protein expression in both LX-2 cells and phHSCs showed a reduced expression of the protein in cells treated with AZD3355 as compared to vehicle controls. See FIGS. 21 and 22.

TABLE 2
mRNA expression of pro-fibrogenic genes in Lx-2 cells after AZD3355 treatment

| | Fibrogenic Gene | | | | | | |
|---|---|---|---|---|---|---|---|
| Treatment | Col 1α1 | αSMA | βPDGF-R | TGF-βR1 | TIMP-1 | TIMP-2 | MMP-2 |
| Group | (p value) | (p value) | (p value) | (p value) | (p value) | (p value) | (p value) |
| AZD3355 | 26 ↓ | 39 ↓ | 41 ↓ | 3 ↑ | 23 ↓ | 26 ↓ | 42 ↓ |
| (30 nM) 48 h | (0.22) | (0.44) | (0.43) | (0.91) | (0.50) | (0.42) | (0.25) |
| AZD3355 | 56 ↓ | 36 ↓ | 24 ↓ | 2 ↓ | 23 ↓ | 25 ↓ | 47 ↓ |
| (100 nM) 48 h | (0.06) | (0.53) | (0.64) | (0.97) | (0.59) | (0.41) | (0.14) |
| AZD3355 | 24 ↓ | 20 ↓ | 11 ↑ | 80 ↓ | 66 ↓ | 59 ↓ | 53 ↓ |
| (30 nM) 72 h | (0.48) | (0.47) | (0.46) | (0.16) | (0.19) | (0.18) | (0.18) |
| AZD3355 | 45 ↓ | 13 ↓ | 14 ↓ | 85 ↓ | 80 ↓ | 73 ↓ | 74 ↓ |
| (100 nM) 72 h | (0.29) | (0.67) | (0.41) | (0.14) | (0.12) | (0.12) | (0.08) |
| Sorafenib | 56↓ | 46 ↓ | 73 ↓ | 30 ↓ | 56 ↓ | 44 ↓ | 89 ↓ |
| (7.5 μM) 48 h | (0.04)* | (0.36) | (0.19) | (0.19) | (0.08) | (0.11) | (0.01)* |
| Sorafenib | 48 ↓ | 69 ↑ | 43 ↓ | 88 ↓ | 92 ↓ | 82 ↓ | 94 ↓ |
| (7.5 μM) 72 h | (0.20) | (0.02)* | (0.06) | (0.13) | (0.08) | (0.07) | (0.03)* |

TABLE 3
mRNA expression of pro-fibrogenic genes in phHSCs after AZD3355 treatment

| | Fibrogenic Gene | | | | | | |
|---|---|---|---|---|---|---|---|
| Treatment | Col 1α1 | αSMA | βPDGF-R | TGF-βR1 | TIMP-1 | TIMP-2 | MMP-2 |
| Group | (p value) | (p value) | (p value) | (p value) | (p value) | (p value) | (p value) |
| AZD3355 | 13 ↓ | 13 ↓ | 2 ↓ | 22 ↓ | 23 ↓ | 0.1 ↑ | 1 ↓ |
| (30 nM) 48 h | (0.42) | (0.33) | (0.28) | (0.34) | (0.14) | (0.96) | (0.93) |
| AZD3355 | 2 ↓ | 12 ↓ | 6 ↓ | 9 ↓ | 9 ↓ | 3 ↑ | 20 ↑ |
| (100 nM) 48 h | (0.85) | (0.37) | (0.76) | (0.60) | (0.53) | (0.85) | (0.18) |
| AZD3355 | 39 ↓ | 32 ↓ | 36 ↓ | 31↓ | 39 ↓ | 16 ↑ | 46 ↓ |
| (30 nM) 72 h | (0.02)* | (0.05)* | (0.008)** | (0.02)* | (0.11) | (0.35) | (0.03)* |
| AZD3355 | 0.1 ↓ | 9 ↓ | 2 ↓ | 4 ↓ | 7 ↓ | 21 ↑ | 11 ↓ |
| (100 nM) 72 h | (0.99) | (0.54) | (0.83) | (0.81) | (0.68) | (0.22) | (0.54) |
| Sorafenib | 6 ↓ | 3 ↑ | 44 ↓ | 17 ↑ | 63 ↓ | 34 ↓ | 9 ↓ |
| (7.5 μM) 48 h | (0.36) | (0.76) | (0.02)* | (0.37) | (0.006)** | (0.11) | (0.40) |
| Sorafenib | 30 ↑ | 20 ↑ | 42 ↓ | 33 ↑ | 67 ↓ | 42 ↓ | 31 ↓ |
| (7.5 μM) 72 h | (0.08) | (0.35) | (0.008)** | (0.09) | (0.01)* | (0.01)* | (0.16) |

TABLE 4
expression of pro-fibrogenic protein
in LX-2 cells after AZD3355 treatment

| | Fibrogenic Gene | | | |
|---|---|---|---|---|
| | | Col1α1 | | |
| | Col1α1 | (Culture | MMP-2 | αSMA |
| Treatment | (Cell lysate) | media) | (cell lysate) | (cell lysate) |
| Group | (p value) | (p value) | (p value) | (p value) |
| AZD3355 | 0 | 21 ↓ | 8 ↓ | 13 ↓ |
| (30 nM) 43 h | (0.99) | (0.29) | (0.81) | (0.55) |
| AZD3355 | 3 ↑ | 23 ↓ | 29 ↓ | 14 ↑ |
| (100 nM) 48 h | (0.90) | (0.17) | (0.29) | (0.57) |
| AZD3355 | 14 ↓ | 32 ↓ | 9 ↓ | 4 ↓ |
| (30 nM) 72 h | (0.29) | (0.10) | (0.55) | (0.65) |
| AZD3355 | 5 ↓ | 11 ↓ | 10 ↑ | 14 ↑ |
| (100 nM) 72 h | (0.69) | (0.00) | (0.65) | (0.14) |
| Sorafenib | 8 ↑ | 18 ↓ | 10 ↓ | 42 ↑ |
| (7.5 μM) 48 h | (0.71) | (0.36) | (0.80) | (0.09) |
| Sorafenib | 24 ↑ | 14 ↑ | 26 ↑ | 31 ↑ |
| (7.5 μM) 72 h | (0.11) | (0.00) | (0.18) | (0.01)* |

TABLE 5
expression of pro-fibrogenic protein in
phHSCs cells after AZD3355 treatment

| | Fibrogenic Gene | | | |
|---|---|---|---|---|
| | | Col1α1 | | |
| | Col1α1 | (Culture | MMP-2 | αSMA |
| Treatment | (Cell lysate) | media) | (cell lysate) | (cell lysate) |
| Group | (p value) | (p value) | (p value) | (p value) |
| AZD3355 | 16 ↓ | 3 ↑ | 6 ↓ | 1 ↓ |
| (30 nM) 48 h | (0.33) | (0.93) | (0.74) | (0.85) |
| AZD3355 | 17 ↓ | 16 ↓ | 1 ↑ | 8 ↓ |
| (100 nM) 48 h | (0.31) | (0.56) | (0.93) | (0.17) |
| AZD3355 | 3 ↓ | 4 ↓ | 4 ↑ | 8 ↑ |
| (30 nM) 72 h | (0.85) | (0.89) | (0.82) | (0.65) |
| AZD3355 | 14 ↓ | 13 ↑ | 3 ↓ | 15 ↑ |
| (100 nM) 72 h | (0.27) | (0.67) | (0.75) | (0.40) |
| Sorafenib | 19 ↓ | 48 ↓ | 4 ↑ | 10 ↓ |
| (7.5 μM) 48 h | (0.16) | (0.06) | (0.83) | (0.22) |
| Sorafenib | 16 ↓ | 67 ↓ | 10 ↓ | 9 ↓ |
| (7.5 μM) 72 h | (0.31) | (0.07) | (0.61) | (0.57) |

### Example 2—Use of AZD3355 for Treatment and Prevention of a Liver Condition or Disease Including NASH as Shown by Liver Slice Assays

### Methods and Materials

Ten human liver pieces were collected from the Mount Sinai biorepository. The samples were prepared by coring the liver with 8 mm cylindrical coring tools. The liver cores were kept in ice cold WE medium, with GlutaMAX supplemental. A liver core was then mounted on a specimen plate with cyanoacrylate adhesive. Liver slices (approximately 200 μm in thickness) were placed in ice cold Krebs-Henesleit buffer in the presence of carbogen gas. There was then a 1 hour pre-incubation in WE GlutaMAX media with gentamicin in 95% O₂ and 5% CO₂ at 37° C. in a humidified rocker chamber to restore ATP levels.

The liver slices were then cultured in WE GlutaMAX media with gentamicin in 95% O₂ and 5% CO₂ at 37° C. in a humidified rocker chamber with slow 70 rpm rocking in the presence of AZD3355 in the amounts of 250 nM, 500 nM, 1000 nM, or 2000 nM, or sorafenib at 1000 nM., or control vehicle (saline water).

After 24 hours, the tissue slices were harvested for either RNA isolation and quantitative PCR for measurements of Col1α1, TNFα, and IL-6 expression, or for histopathology and hematoxylin and eosin (H&E) staining as described in Example 3.

### Results

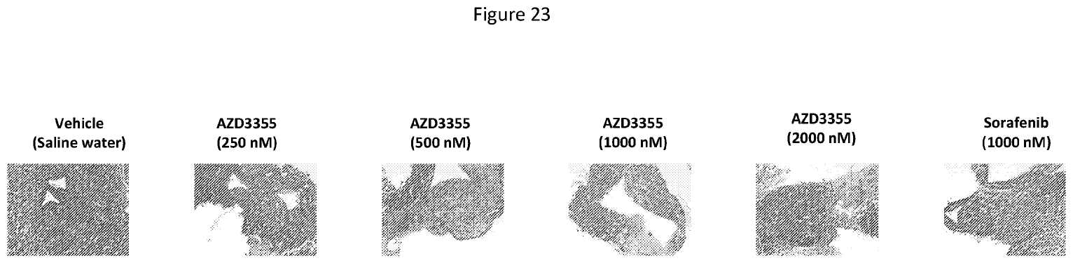

Histopathology and H&E staining showed that the liver cells viability did not change across AZD3355 or sorafenib treatment. See FIG. 23.

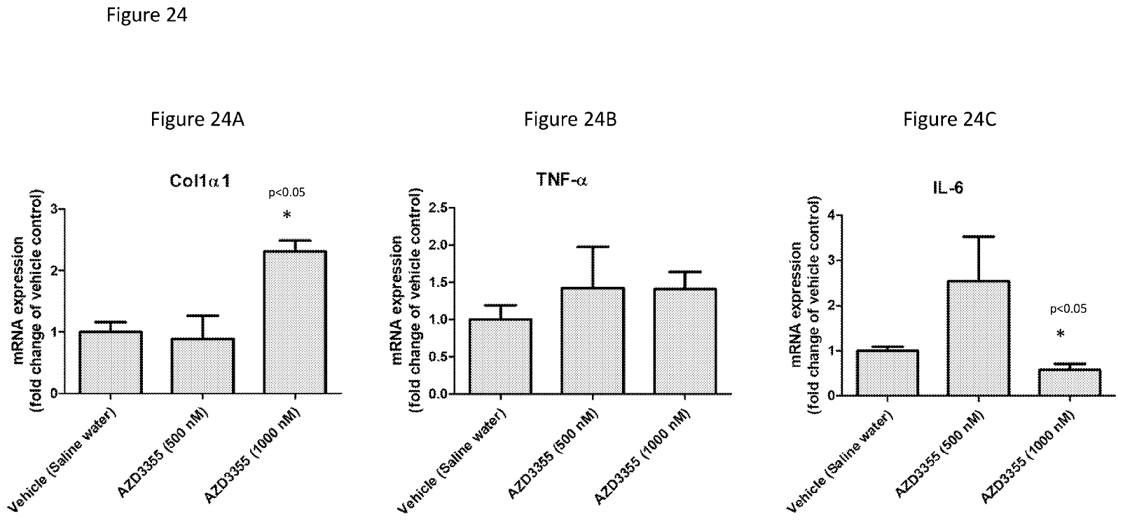

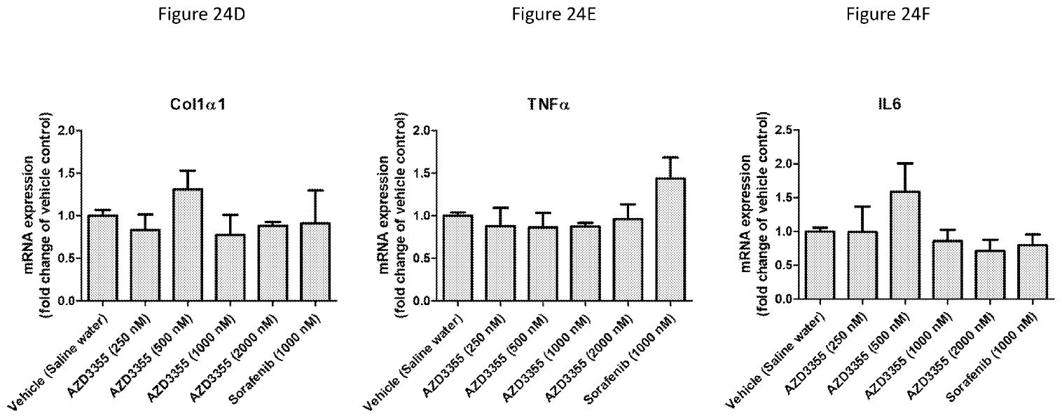

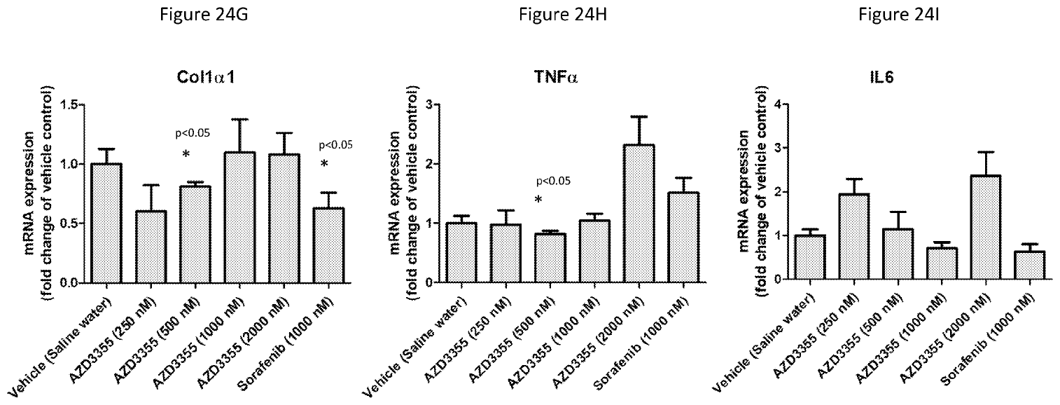

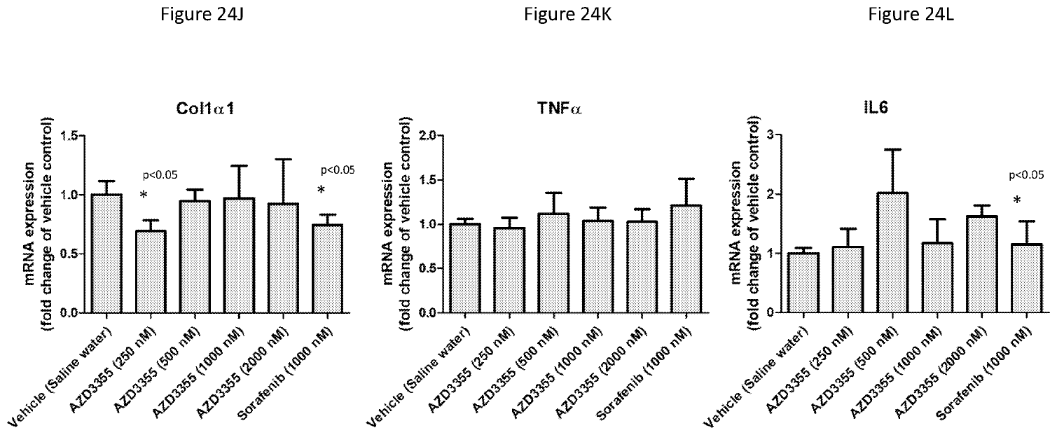

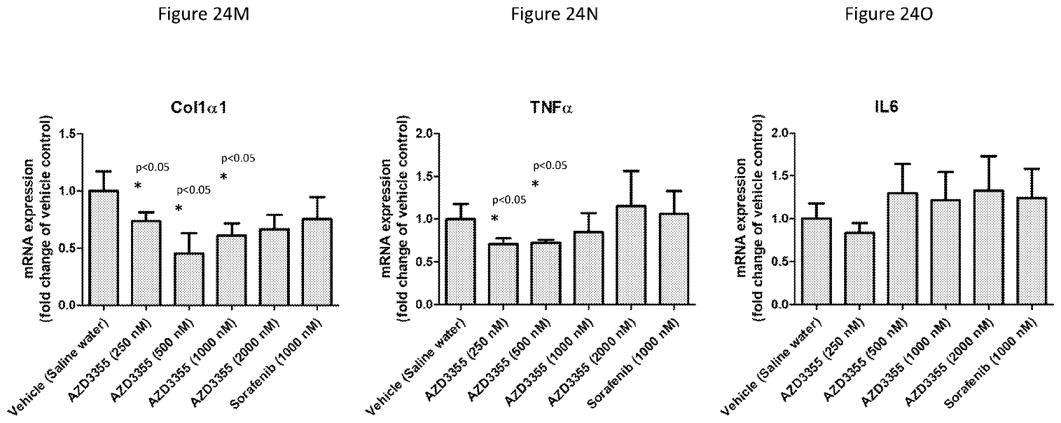

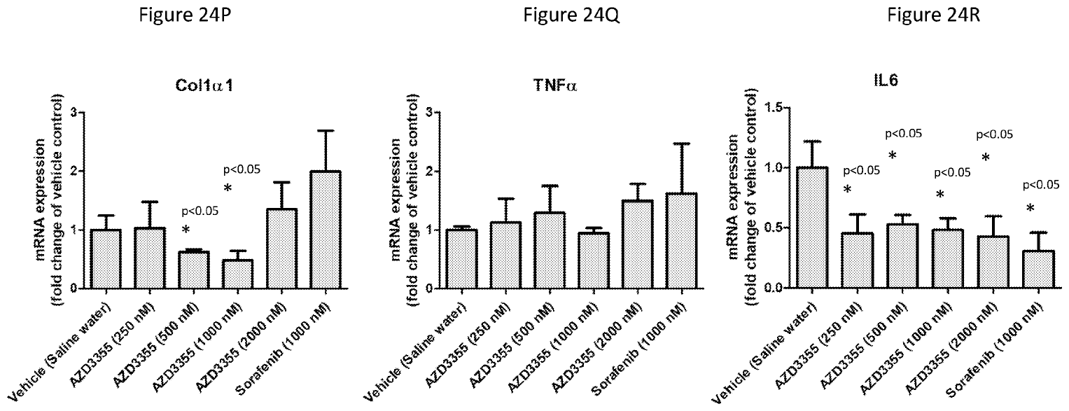

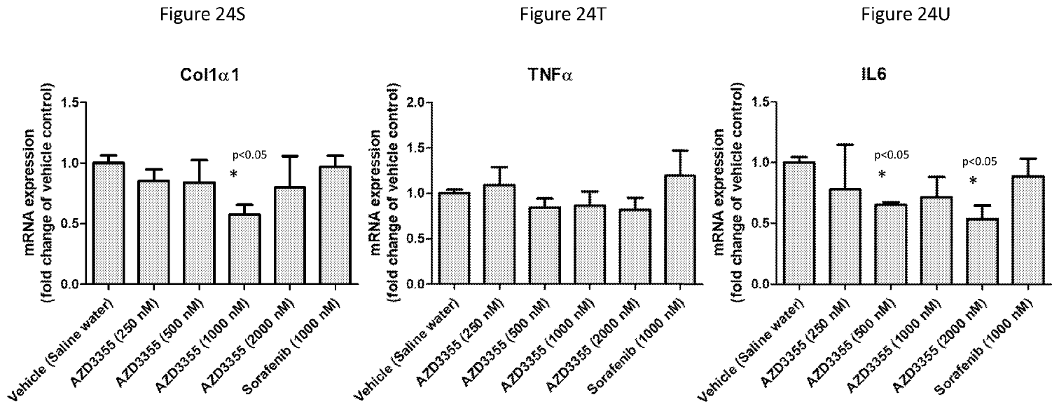

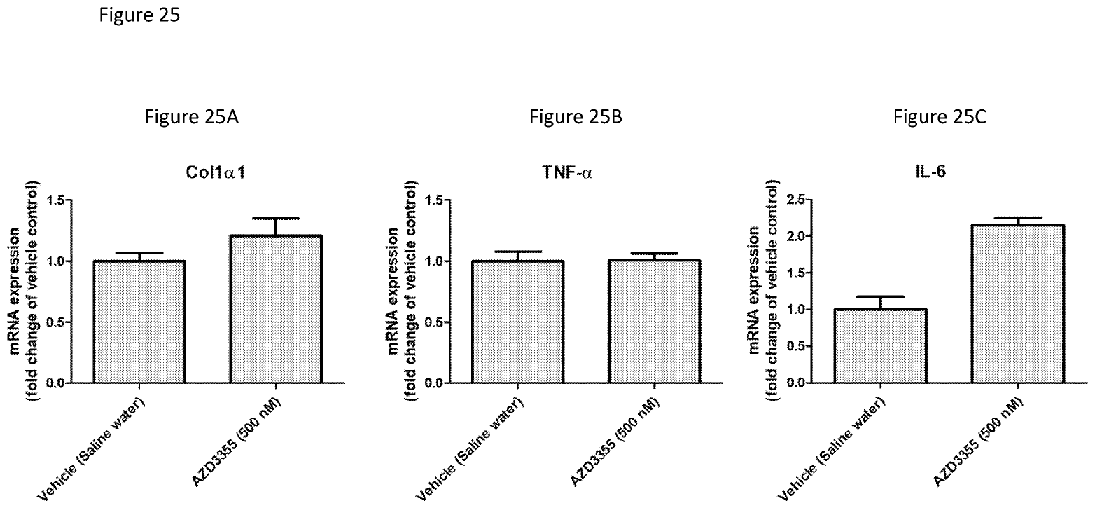

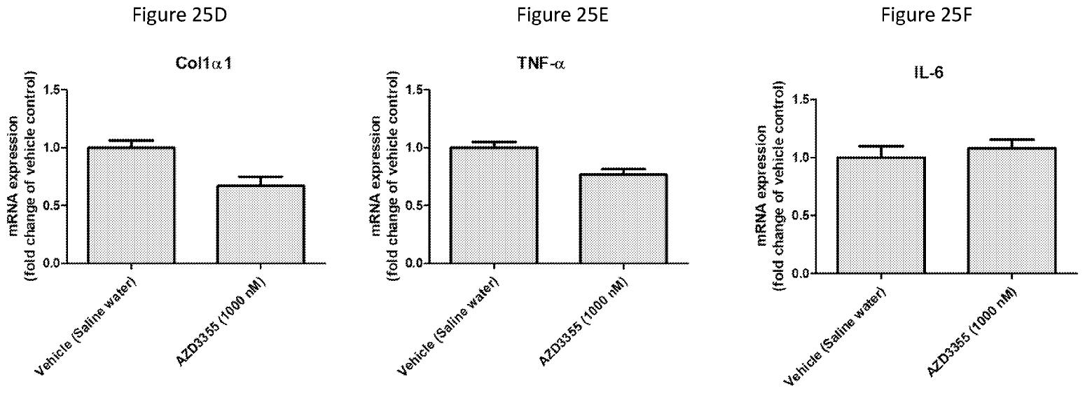

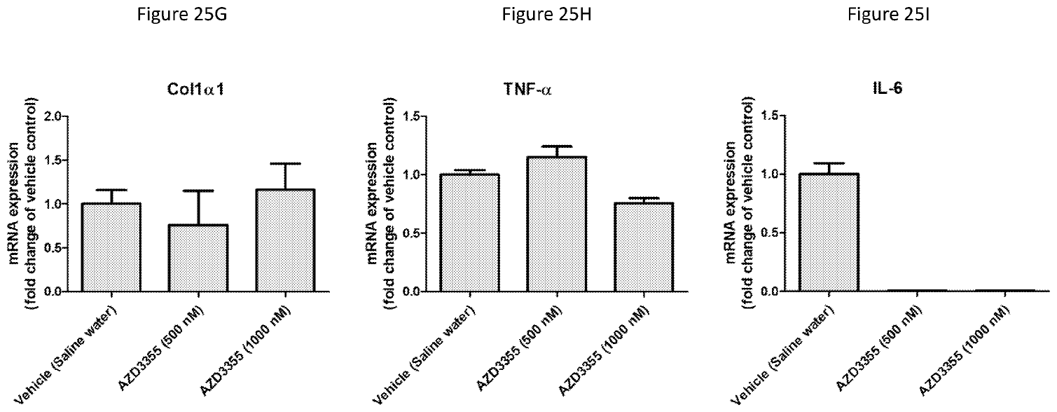

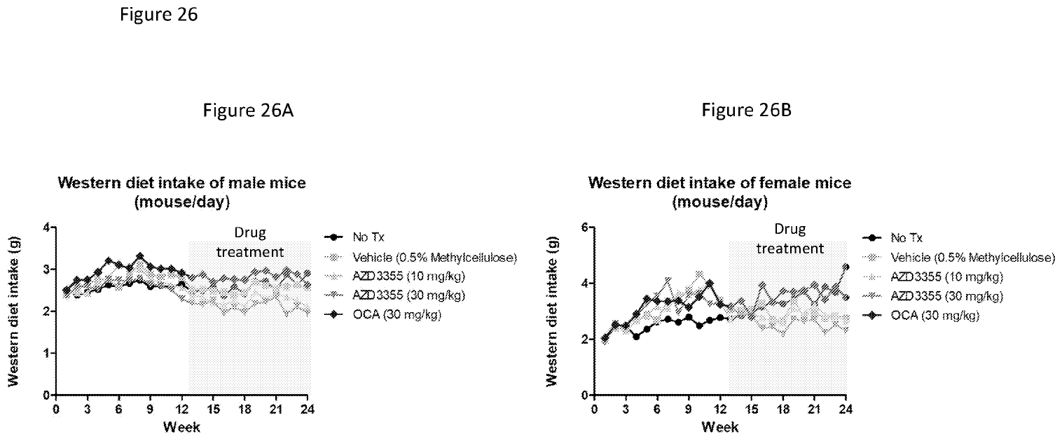

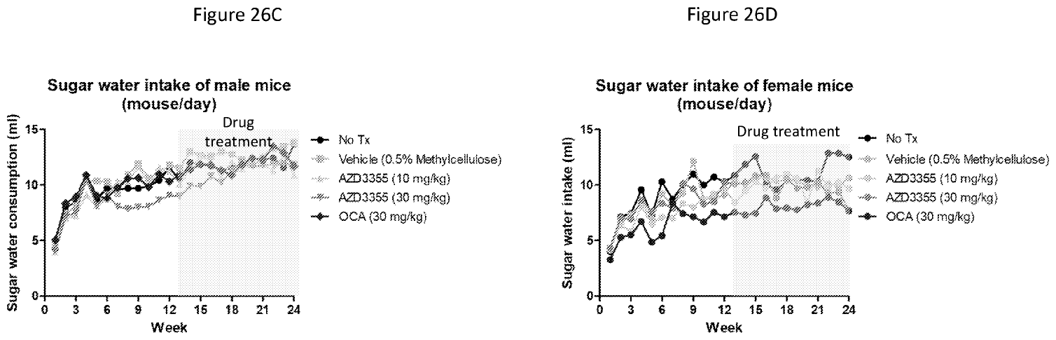

qPCR showed that in most of the ten samples, at least one of the pro-fibrotic genes was down-regulated after 24 hours cultured in AZD3355. See FIGS. 24 and 25, and Table 6. In many cases the down-regulation was significant. See FIGS. 24C, 24G, 24H, 24J, 24M, 24N, 24P, 24R, 24S, 24U, 25D, 25E, 25H, and 25I.

TABLE 6
expression of pro-fibrogenic genes in liver slices after AZD3355 treatment

| | Collagen1α1 (p value) | | | | | TNF-α | | |
|---|---|---|---|---|---|---|---|---|
| | AZD3355 | | | | Sorafenib | AZD3355 | | |
| Liver | 250 | 500 | 1000 | 2000 | 1000 | 250 | 500 | 1000 |
| Sample | nM | nM | nM | nM | nM | nM | nM | nM |
| AZ1 | x | 11 ↓ | 131 ↑ | x | x | x | 42 ↑ | 41 ↑ |
| | | | (<0.05)* | | | | | |
| AZ2 | 17 ↓ | 31 ↑ | 22 ↓ | 12 ↓ | 9 ↓ | 12 ↓ | 14 ↓ | 13 ↓ |
| AZ3 | 40 ↓ | 19 ↓ | 10 ↑ | 8 ↑ | 37 ↓ | 2 ↓ | 18 ↓ | 4 ↑ |
| | | (<0.05)* | | | (<0.05)* | | (<0.05)* | |
| AZ4 | 31 ↓ | 5 ↓ | 31 ↑ | 22 ↓ | 25 ↓ | 4 ↓ | 12 ↑ | 4 ↑ |
| | (<0.05)* | | | | (<0.05)* | | | |
| AZ5 | 26 ↓ | 55 ↓ | 39 ↓ | 33 ↓ | 25 ↓ | 30 ↓ | 27 ↓ | 15 ↓ |
| | (<0.05)* | (<0.05)* | (<0.05)* | | | (<0.05)* | (<0.05)* | |
| AZ6 | 3 ↑ | 38 ↓ | 52 ↓ | 35 ↑ | 99 ↑ | 13 ↑ | 29 ↑ | 5 ↓ |
| | | (<0.05)* | (<0.05)* | | | | | |
| AZ7 | 15 ↓ | 16 ↓ | 42 ↓ | 20 ↓ | 3 ↓ | 9 ↑ | 16 ↓ | 14 ↓ |
| | | | (<0.05)* | | | | | |
| ev417 | x | 21 ↑ | x | x | x | x | 1 ↑ | x |
| ev422 | x | x | 33 ↓ | x | x | x | x | 23 ↓ |
| | | | (<0.05)* | | | | | (<0.05)* |
| ev430 | x | 24 ↓ | 16 ↑ | x | x | x | 15 ↑ | 24 ↓ |
| | | | | | | | | (<0.05)* |

| | TNF-α | | IL-6 (p value) | | | | |
|---|---|---|---|---|---|---|---|
| | AZD3355 | Sorafenib | AZD3355 | | | | Sorafenib |
| Liver | 2000 | 1000 | 250 | 500 | 1000 | 2000 | 1000 |
| Sample | nM | nM | nM | nM | nM | nM | nM |
| AZ1 | x | x | x | 154 ↑ | 42 ↓ | x | x |
| | | | | | (<0.05)* | | |
| AZ2 | 4 ↓ | 44 ↑ | 1 ↓ | 59 ↑ | 14 ↓ | 29 ↓ | 20 ↓ |
| AZ3 | 13 ↑ | 52 ↑ | 94 ↑ | 14 ↑ | 29 ↓ | 136 ↑ | 36 ↓ |
| AZ4 | 3 ↑ | 21 ↑ | 11 ↑ | 102 ↑ | 17 ↑ | 62 ↑ | 15 ↑ |
| | | | | | | | (<0.05)* |
| AZ5 | 15 ↑ | 6 ↑ | 16 ↓ | 30 ↑ | 21 ↑ | 33 ↑ | 24 ↑ |
| AZ6 | 50 ↑ | 62 ↑ | 55 ↓ | 47 ↓ | 52 ↓ | 57 ↓ | 69 ↓ |
| | | | (<0.05)* | (<0.05)* | (<0.05)* | (<0.05)* | (<0.05)* |
| AZ7 | 18 ↓ | 19 ↑ | 22 ↓ | 35 ↓ | 28 ↓ | 46 ↓ | 11 ↓ |
| | | | | (<0.05)* | | (<0.05)* | |
| ev417 | x | xx | x | 115 ↑ | x | x | x |
| | | | | (<0.05)* | | | |
| ev422 | x | x | x | x | 8 ↑ | x | x |
| ev430 | x | x | x | 99 ↓ | 99 ↓ | x | x |
| | | | | (<0.05)* | (<0.05)* | | |

### Example 3—Use of AZD3355 for Treatment and Prevention of a Liver Condition or Disease Including NASH as Shown by In Vivo NASH Mouse Model

### Methods and Materials

Animals:

The experimental protocol was reviewed and approved by the Institutional Animal Care and Use Committee (IACUC) at Icahn School of Medicine at Mount Sinai, N.Y. Male and Female C57BL/6J mice (age: 6 weeks; weight: 20-25 g) were purchased from Jackson Laboratories (Farmington, Conn.) Animals were housed in a 12 hours light-12 hours dark cycle in the animal facility at Icahn School of Medicine at Mount Sinai, N.Y. and handled following guidelines for the care and use of laboratory animals Male and female mice were maintained in separate cages.

Carbon Tetrachloride, Western Diet and Sugar Water:

Carbon tetrachloride (CCl4) was purchased from Sigma-Aldrich, MO. CCl₄ was freshly dissolved in corn oil at final concentration of 5% before injection. The final dose of 100% CCl₄ was 0.2 μl/g of body weight of mice via intra-peritoneal route once/week were introduced starting from week one parallel to the western diet-sugar water fed for a total period of 24 weeks.

Western diet containing 21.2% fat (42% Kcal), 41% sucrose and 1.25% cholesterol by weight was purchased from Envigo, WI (Teklad Custom diet Cat #TD.120528). Sugar water solution containing 18.9 g/L D-(+)-Glucose (Sigma-Aldrich, MO) and 23.1 g/L D-(−)-Fructose (Sigma-Aldrich, MO) dissolved in normal water and filter sterilized. Exchange the western diet and sugar water in each cage were twice/week to avoid microbial contaminations. During change of food and water we have been measured the amount of food and water intake and the data was collected.

AZD3355 and OCA Small Molecules and Methylcellulose:

AZD3355 small molecule (FW 141.08) was supplied by AstraZeneca and for vehicle methylcellulose (4,000 cP) was commercially purchased from Sigma-Aldrich, MO. The positive control drug, Obeticholic acid (OCA; FW 420.63) was purchased from ApexBio (Houston, Tex.). 0.5% methylcellulose was made in ultrapure water and keep at 4° C. during whole period of experiment. Two different concentrations of AZD3355 solutions (10 mg/ml and 30 mg/ml (w/v)) were made in 0.5% methylcellulose just before each gavaging and unused diluted drugs were discarded. 30 mg/kg concentration of OCA was also made fresh each week and aliquoted and stored at −20° C. freezer. One aliquot was taken from each day and unused diluted OCA are discarded.

No Treatment (No Tx)—12 Weeks Group:

At beginning of week 13, mice were distributed into five different groups. Mice of No Tx-12 weeks were sacrificed, and blood samples collected (through IVC) for serum preparation. Whole liver from the animals was excised, cleaned in 1×PBS, weight recorded, and photographed. The livers were examined to determined fibrosis/tumor(s) developments by eye and the data was recorded. Spleens were excised and weights recorded. Liver and blood serum samples were stored at −80° C. for further analysis.

0.5% Methylcellulose, AZD3355, OCA Dosing and No Tx Control—24 Weeks groups:

Four separate groups of mice were given either 0.5% methylcellulose (as Vehicle) or AZD3355 at 10 mg/kg (low dose) body weight of mice or 30 mg/kg (high dose) body weight of mice by twice daily (BID (5 days/week)) and OCA at 30 mg/kg body weight of mice by daily (QD (5 days/week)) through oral route for up to next 12 weeks (week 13 to week 24). Themlast group of mice were not given any drug or vehicle treatment, as No Tx-24 weeks and maintained in parallel with treatment/vehicle groups. Usually all doses were given at early morning and the second daily dose of AZD3355 at evening (10 hours of intervals). All animals were closely monitored the health conditions and behavior.

End of the AZD3355 or OCA Small Molecule and Vehicle Treatment:

The male and female mice in vehicle (0.5% methylcellulose), AZD3355 10 mg/kg dosing, AZD3355 30 mg/kg dosing, OCA 30 mg/kg dosing and No Tx control—24 weeks groups were sacrificed at end of week 24. Blood samples were collected (through IVC) for serum preparation. Whole liver from animal was excised, cleaned in 1×PBS, weight recorded, and photographed. The livers were examined to determined fibrosis/tumor(s) developments by eye and the data was recorded. Spleens were excised and recorded weights. Liver and blood serum samples were stored at −80° C. for further analysis.

Blood Serum Preparation:

Collected blood samples were kept in room temperature for 30 minutes for clotting. Serum was collected after centrifugation the blood samples at 2000 Xg for 10 minutes at 4° C. and stored at −80° C. until analysis.

Liver Enzymes and Lipid Panel Analysis:

Aspartate aminotransferase (AST), alanine aminotransferase (ALT), cholesterol and triglycerides were measured from blood serum in ARCHITECT c16000 Clinical Chemistry Analyzer (Abbott Diagnostics, MA) in Mount Sinai Clinical Chemistry Laboratory facility according to the manufacturer's instruction.

Histopathological Analysis in Liver Tissue:

A piece of tissue from large liver lobe was fixed in 10% Formalin Buffered (Astral Diagnostics Incorporated NJ) embedded in paraffin and microtome sectioning were performed. Slides containing tissue sections were baked at 60° C. for 1 hour and re-hydrated through xylene followed by graded ethanol (100%, 95%, 85% and 70%) into distilled water and processed in either Picrosirius red/Fast green or hematoxylin and eosin staining.

Picrosirius Red/Fast Green Staining and Morphometric Measurement of Collagen:

For collagen staining, re-hydrated slides were stained for one hour in saturated picric acid with 0.1% Sirius Red (Direct Red-80; Sigma-Aldrich, MO) followed by counterstained with 0.01% Fast Green (Sigma-Aldrich, MO) for one more hour. The slides were removed from stain, rinsed in water and rapidly dehydrated through graded ethanol (70%, 85%, 95% and 100%) followed through xylene and finally cover slipped in Permount (ThermoFisher Scientific, NJ: Cat #SP15-500). Whole slide with staining sections were digitized scanned in Aperio AT2 digital scanner (Leica Biosystems Inc., IL). The image from each scanned section was randomly saved as 5× zoom level (3 images/section) in Aperio ImageScope [v12.4.0.5043] (Leica Biosystems Inc., IL) histopathological diagnostic software. A total of 6 sections/animal were stained and 3 images from each section (total 18 pictures/animal) were evaluated using BIOQUANT image analysis software (Bioquant Image Analysis Corporation, TN) to quantify collagen accumulation in liver tissue.

Hematoxylin and Eosin (H&E) Staining:

A total of 2 sections/animal were stained with H&E staining performed by standard protocol.

Histopathological Scoring of Liver Sections:

Steatosis, hepatocyte ballooning, lobular inflammation, portal inflammation and fibrosis of picroserious red/fast green and H&E stained liver section were scored according to the NASH Clinical Research Network (NASH CRN) scoring system in a blinded fashion by an expert hepato-pathologist in Icahn School of Medicine at Mount Sinai. NAFLD activity score (NAS) was calculated according to Brunt criteria and range from 0-8. NAS was calculated by the sum of scores of steatosis (0-3), hepatocyte ballooning (0-2) and lobular inflammation (0-3). NAS score of >5.0 strongly correlated with “definite-NASH” whereas <3 correlated with “not-NASH”. Fibrosis (0-4) or portal inflammation (0-3) scores were assessed separately and not included in NAS.

Fibrogenic Gene Expression in Liver Tissue by RT-qPCR:

mRNA expression of following fibrogenic genes were assessed:

### 1. Collagen1α1 (Col1α1);

### 2. Alpha Smooth Muscle Actin (αSMA);

### 3. Beta-type Platelet-Derived Growth Factor-Receptor (βPDGF-R);

### 4. Transforming Growth Factor Beta-Receptor 1 (TGFβ-R1);

### 5. Tissue Inhibitor of Metalloproteinases-1 (TIMP-1);

### 6. Tissue Inhibitor of Metalloproteinases-2 (TIMP-2); and

### 7. Matrix Metalloproteinase-2 (MMP-2).

Total RNA was extracted from approximately 30 mg of liver tissue using RNeasy Mini Kit (Qiagen, CA). 1 μg of total RNA was used for reverse transcription with ‘RNA to cDNA EcoDry Premix (Double Primed) Kit’ (Clontech, CA). Expression of fibrogenic genes were measured by qPCR using custom designed primers (Sigma-Aldrich, MO) and iQ SYBR Green Supermix (Bio-Rad, CA) on a LightCycler 480 II (Roche Diagnostics Corporation, IN) instrument. Glyceraldehyde-3-phosphate dehydrogenase (GAPDH) were used as housekeeping gene to determine the relative expression of fibrogenic genes.

Fibrogenic Protein Expression in Liver Tissue by Western Blot and Densitometric Analysis:

Expression of following fibrogenic proteins were assessed:

### 1. Collagen1α1 (Col1α1)

### 2. Alpha Smooth Muscle Actin (αSMA)

Total protein was extracted from approximately 30 mg of liver tissue using RIPA buffer (50 mM Tris-HCl pH8.0, 150 mM NaCl, 1% IGEPAL, 0.5% Sodium Deoxycholate and 0.1% SDS) along with Pierce Protease Inhibitor Mini Tablets, EDTA-Free (Thermo Scientific, IL) and Phosphatase inhibitor Cocktail (Thermo Scientific, IL). Lysate were homogenized in presence of one 5 mm Stainless Steel Beads (Qiagen, Germantown, Md.) using a TissueLyser LT homogenizer (Qiagen, Germantown, Md.) at 50 Hz/second for 2 minutes. Total protein was collected from the homogenate (middle aqueous phase) after centrifugation at 14000 rpm for 10 minutes. Total protein was measured by Bradford colorimetric assay using Protein Assay Dye Reagent Concentrate (Bio-Rad, CA). 15 μg of proteins were loading in NuPAGE 4-12% Bis-Tris gel (Thermo Scientific, IL). After transferred, the protein bands to PVDF membrane the bands were blocked with 5% non-fat milk in 1×PBS. The primary antibodies used for probed the respective protein bands were rabbit anti-Collagen1 (Bioss, MA) and rabbit anti-αSMA (abcam, MA) and mouse anti-GAPDH (Millipore, CA). After hybridized with HRP conjugated secondary antibody (either Goat anti-rabbit HRP (Jackson ImmunoResearch Laboratories, PA) or anti-mouse IgG-HRP (Cell Signaling Technology, MA)) the membrane was treated with Immobilon Western Chemiluminescent HRP substrate (Millipore, MA) and the signals were captured with Amersham Imager 6000 (GE Healthcare, PA). 210 kD of Collagen1α1 and 42 kD of αSMA bands were clearly recognized by respective antibody. A 37 kD band of GAPDH was probed as loading control. For densitometric measurement of the protein bands, images were exported and analyzed using ImageJ 1.50f software and bands were normalized to the loading control, GAPDH.

Statistical Data Analysis:

The data analysis was accomplished by using GraphPad Prism v7.4 statistical software (GraphPad Software, Inc., CA). Standard error mean (±SEM) was calculated according to student t-test or unpaired two tailed Mann-Whitney test where Gaussian distribution is non-parametric. Unless otherwise specified, p values <0.05 were considered statistically significant (*=p<0.05 vs vehicle group).

### Results

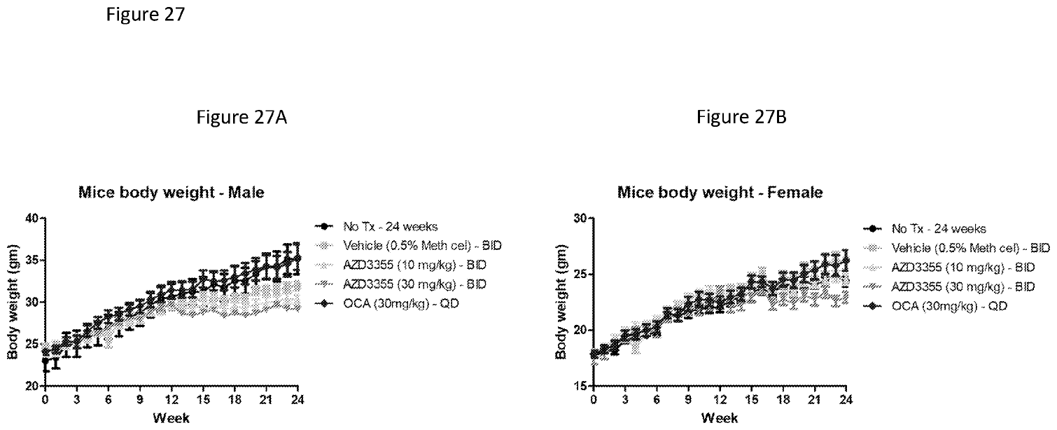

Each group of NASH model mice was weighed throughout the 12-week course of treatment. The body weight of all of the mice was stable during AZD3355 treatment indicating that there were no toxic effects of the drug (FIG. 27). There was a significant reduction in body weight of both the male and female NASH model mice treated with 30 mg/kg of AZD3355 as compared to vehicle treated mice in both groups. The reduction in the male mice was seen at the second week of treatment (see FIG. 27A, *=p<0.05) and in the third week of treatment in the female mice (see FIG. 27B, *=p<0.05).

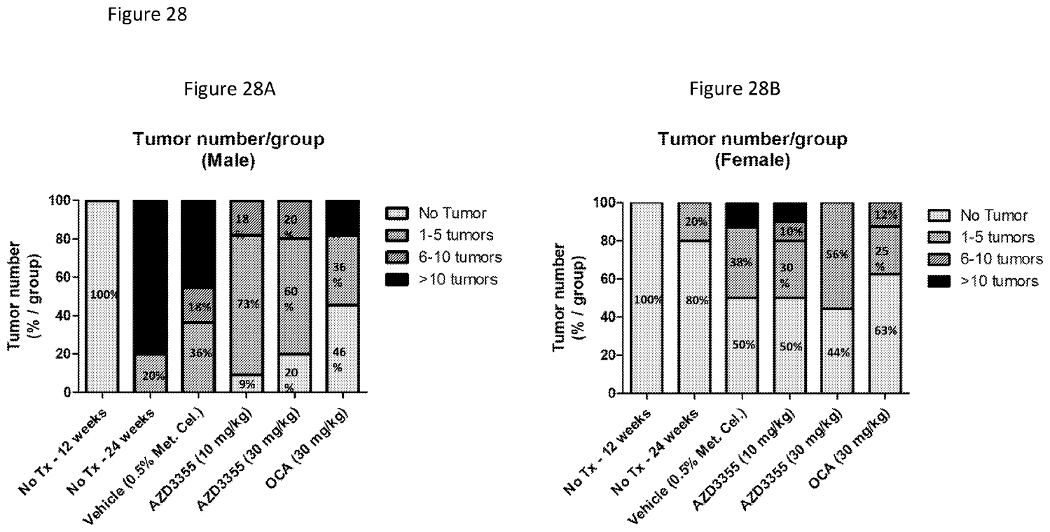

The tumor development in the NASH model mice was also assessed at 12 weeks and then at 24 weeks for each group of mice, untreated, treated with vehicle, treated with AZD3355 and treated with OCA. In both male and female NASH model mice, treatment with either AZD3355 at either dose or OCA reduced tumor development in the liver (FIG. 28).

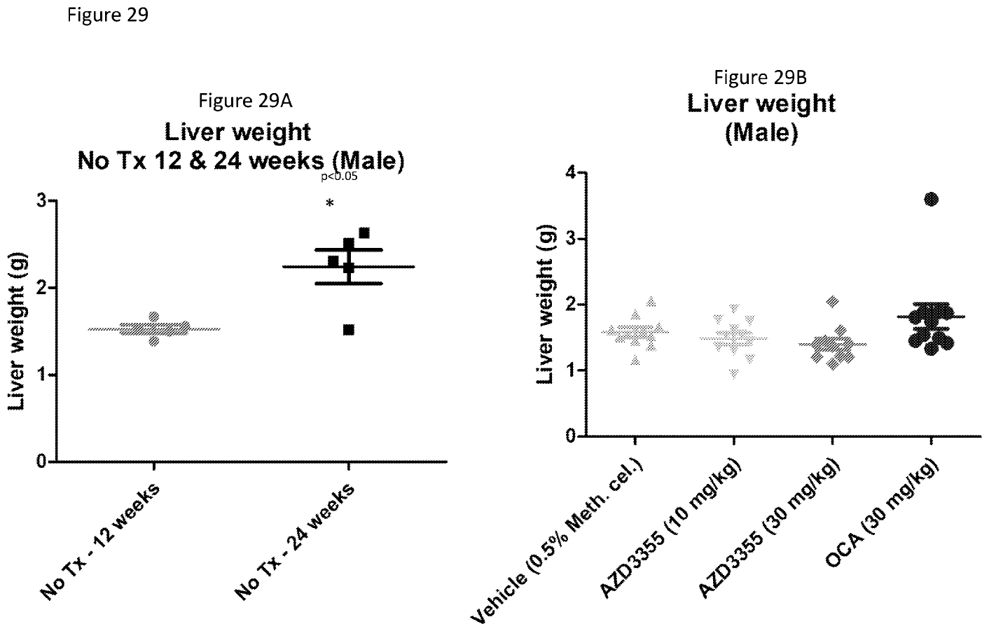

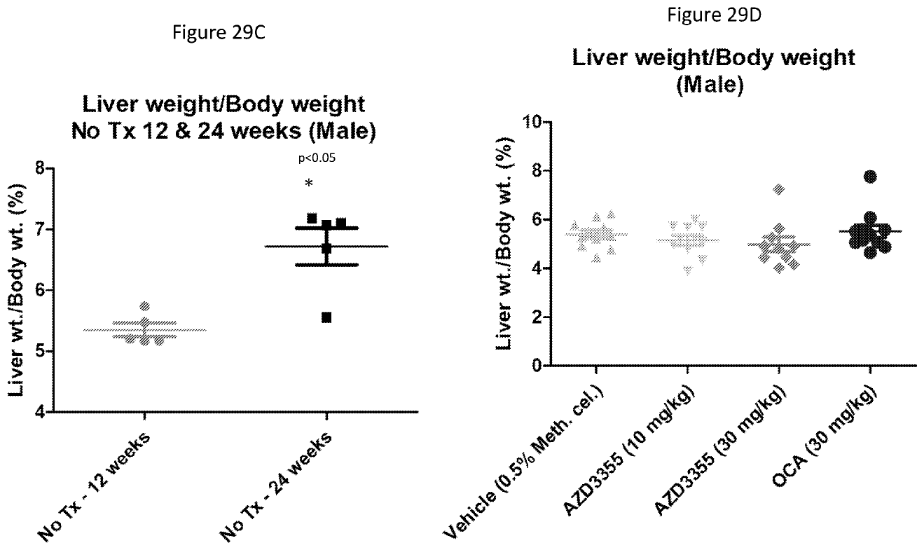

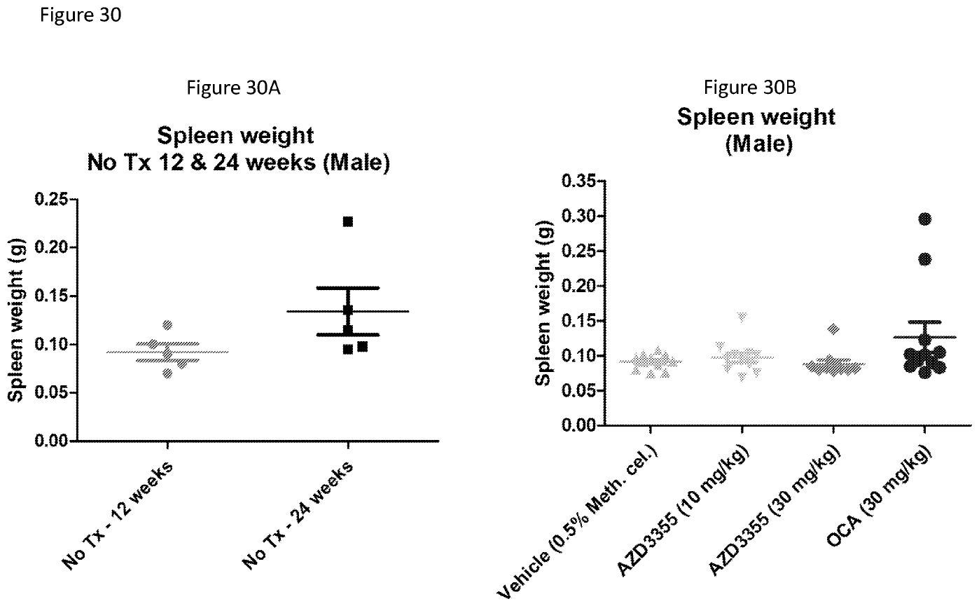

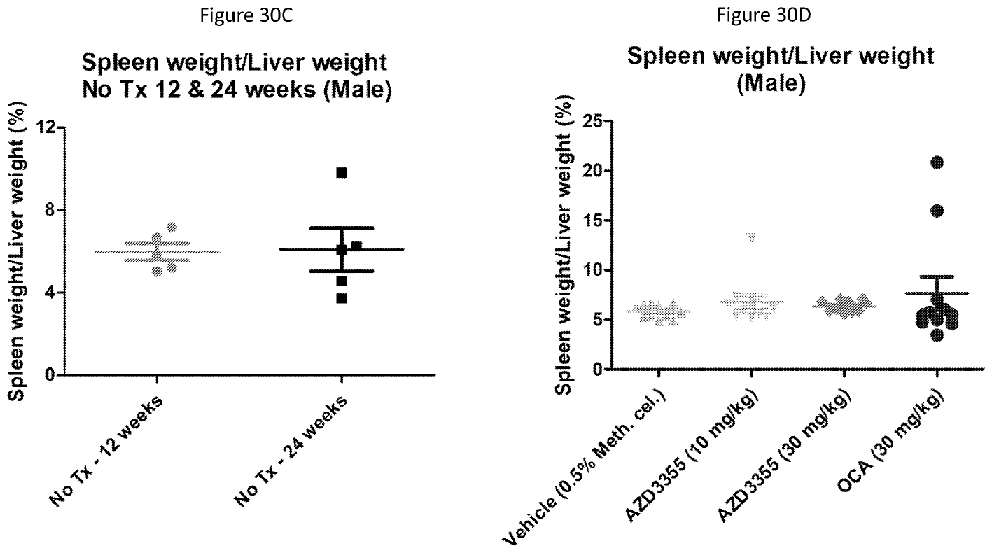

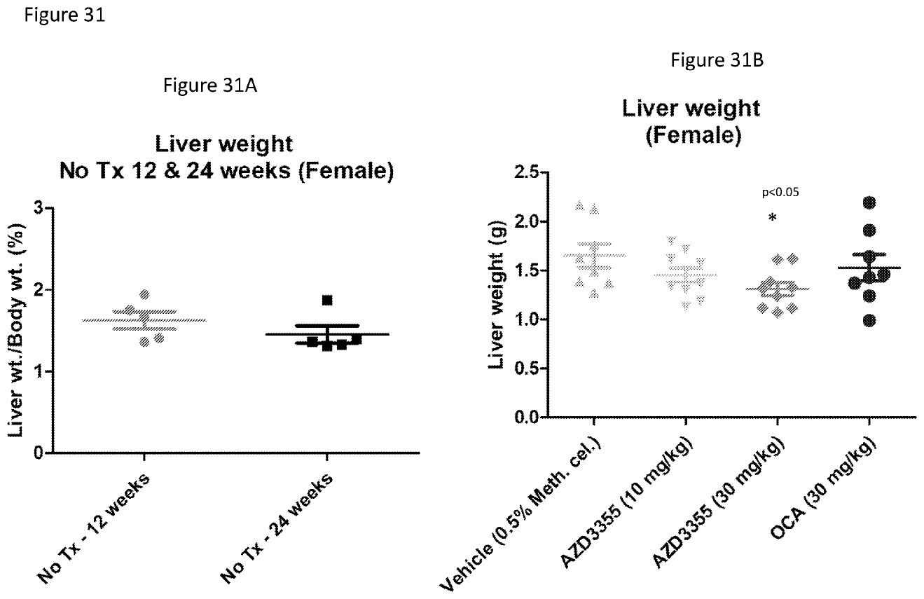

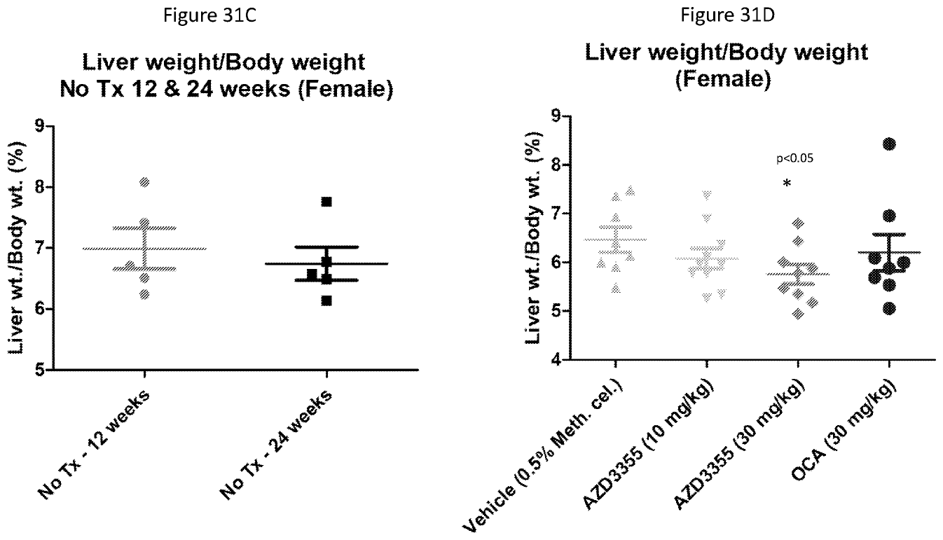

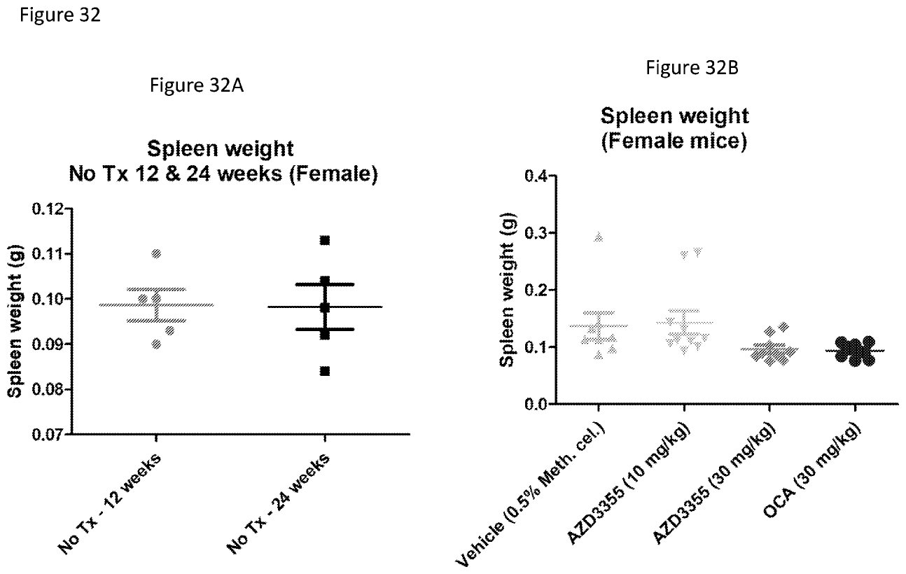

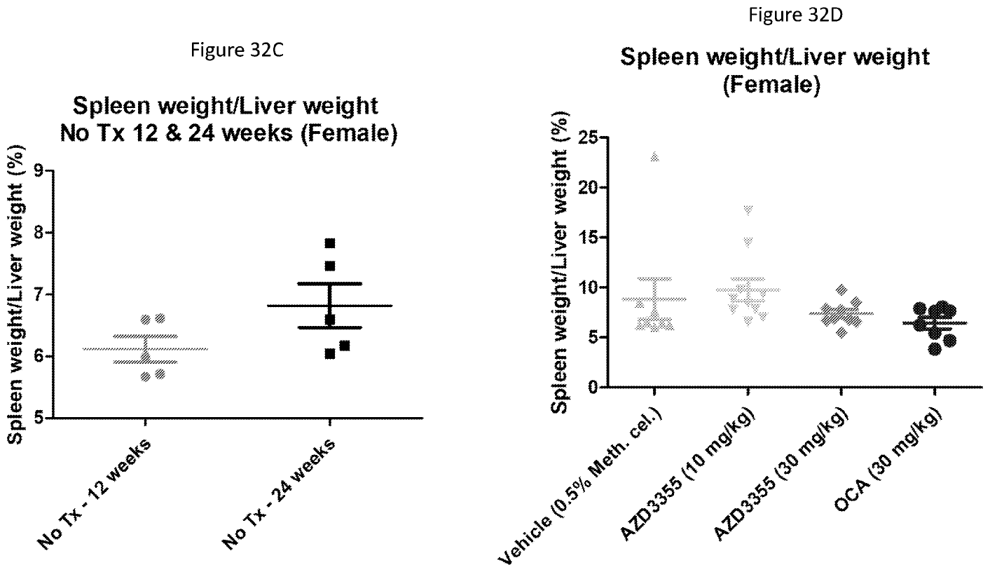

Additionally, liver weigh, liver/body weight ratio, and spleen weight were improved in both male and female NASH model mice treated with AZD3355 at both dosages. See FIGS. 29-32.

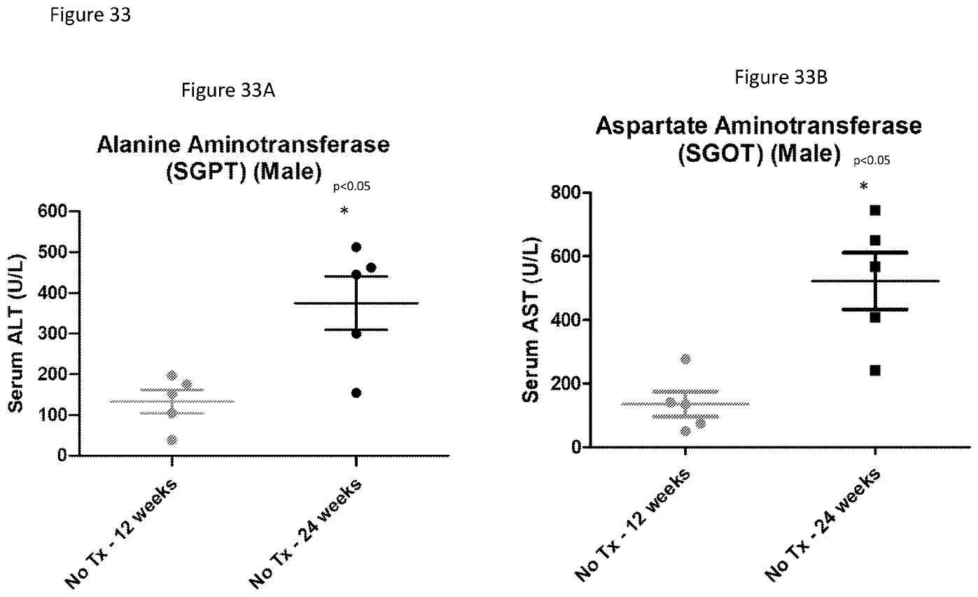

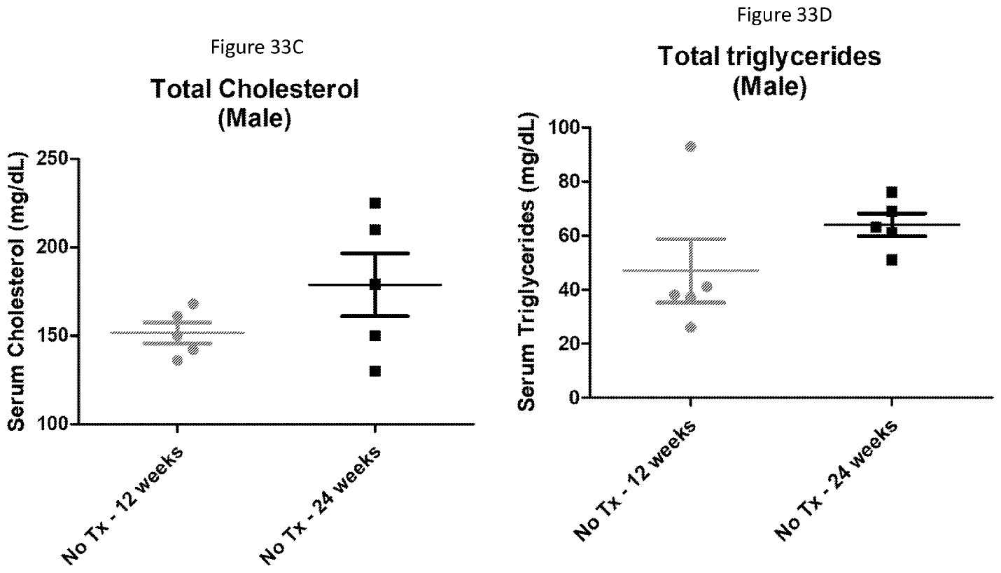

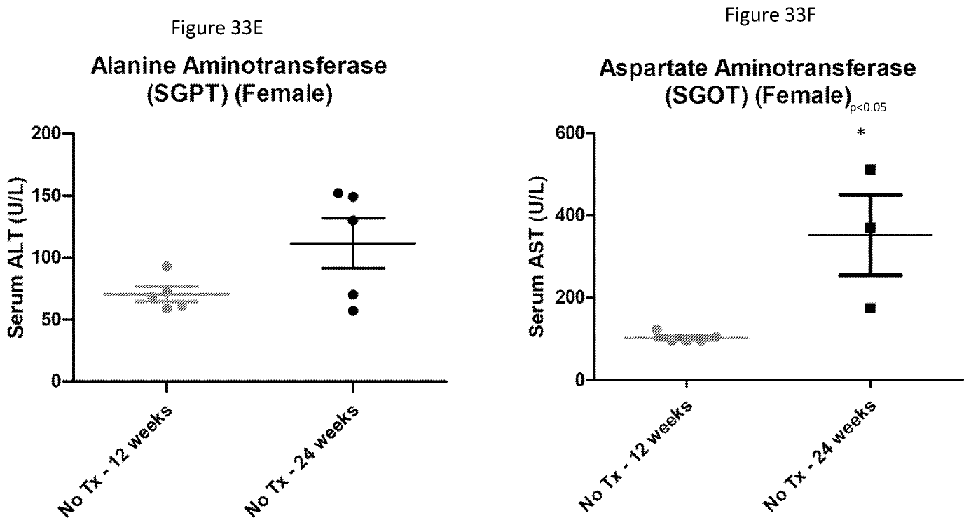

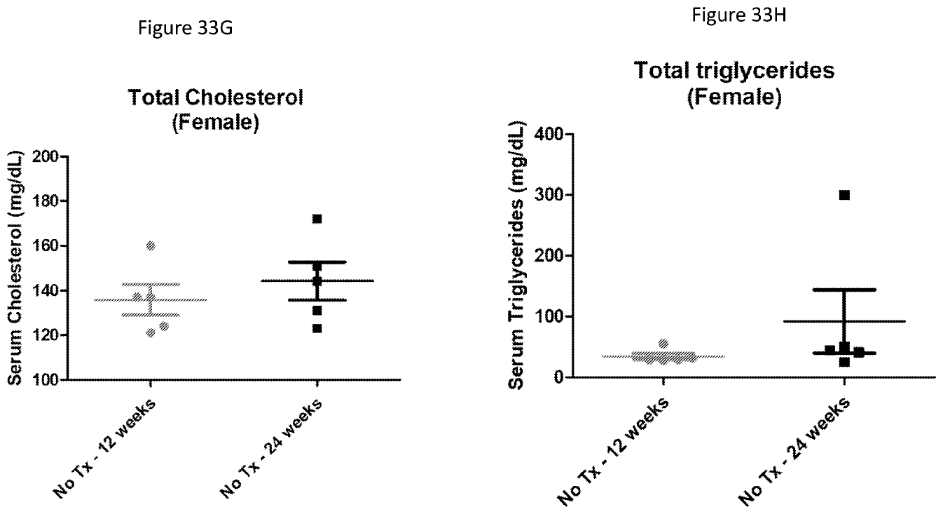

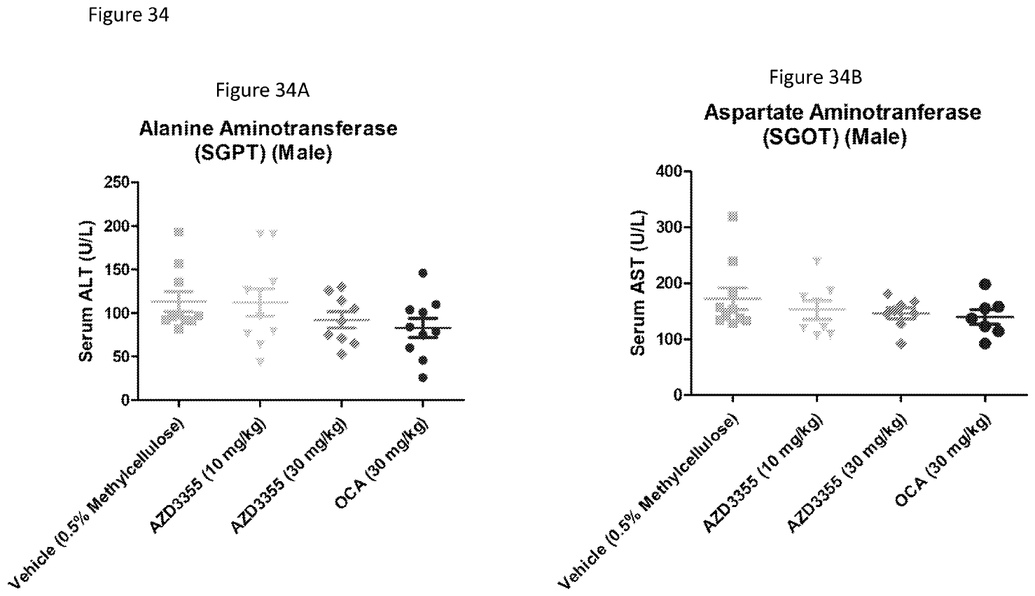

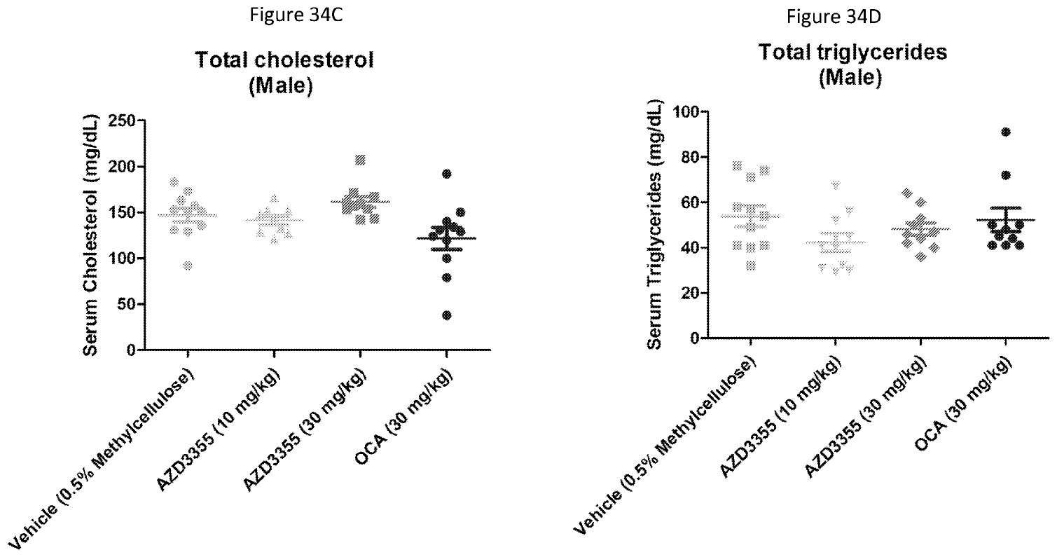

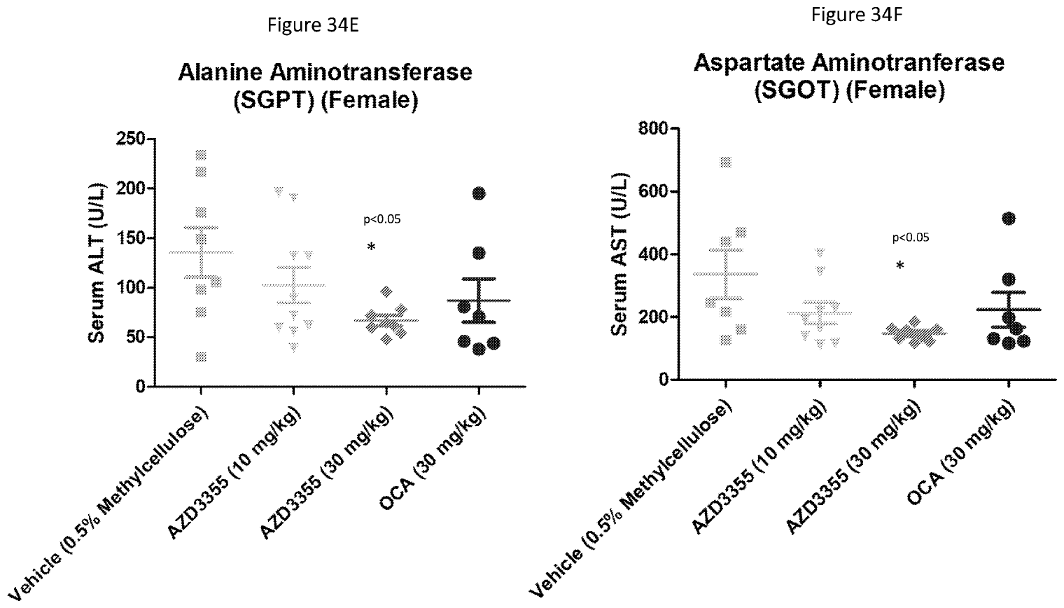

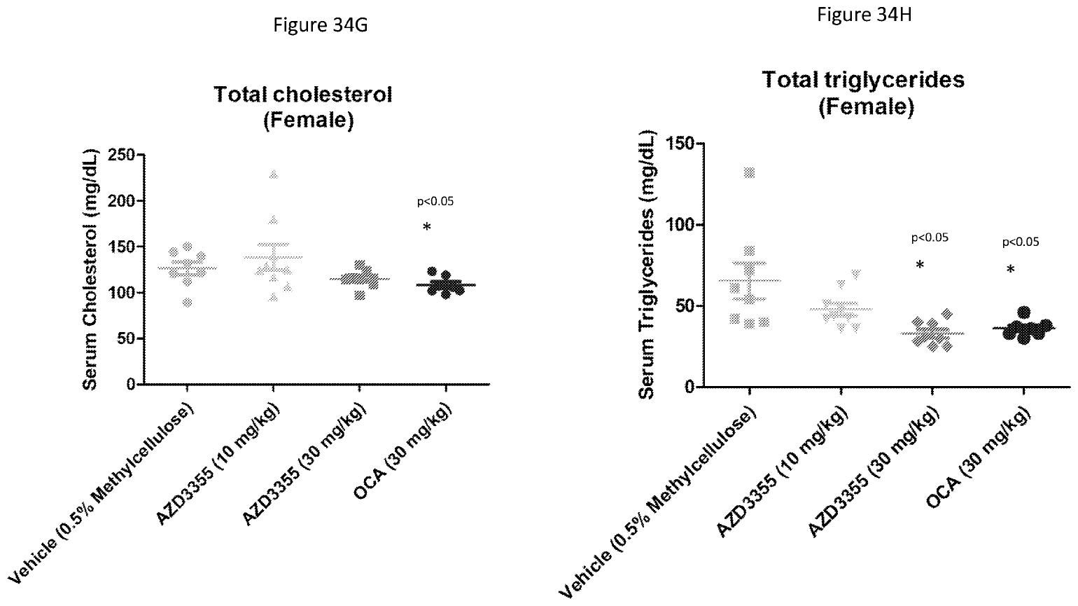

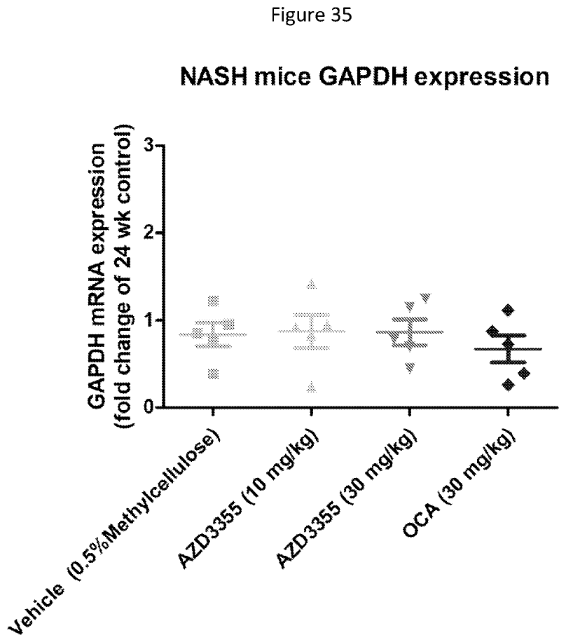

NASH mice, both male and female, have elevated liver enzymes at 24 weeks of NASH induction versus 12 weeks. These enzymes include alanine aminotransferase (SGPT) and aspartate aminotransferase (SGOT). The NASH mice also had elevated total cholesterol and triglycerides at 24 weeks as compared to 12 weeks. See FIG. 33. Treatment with AZD3355 or OCA for 12 weeks improved the necro-inflammatory activity in both male and female NASH model mice. See FIG. 34.

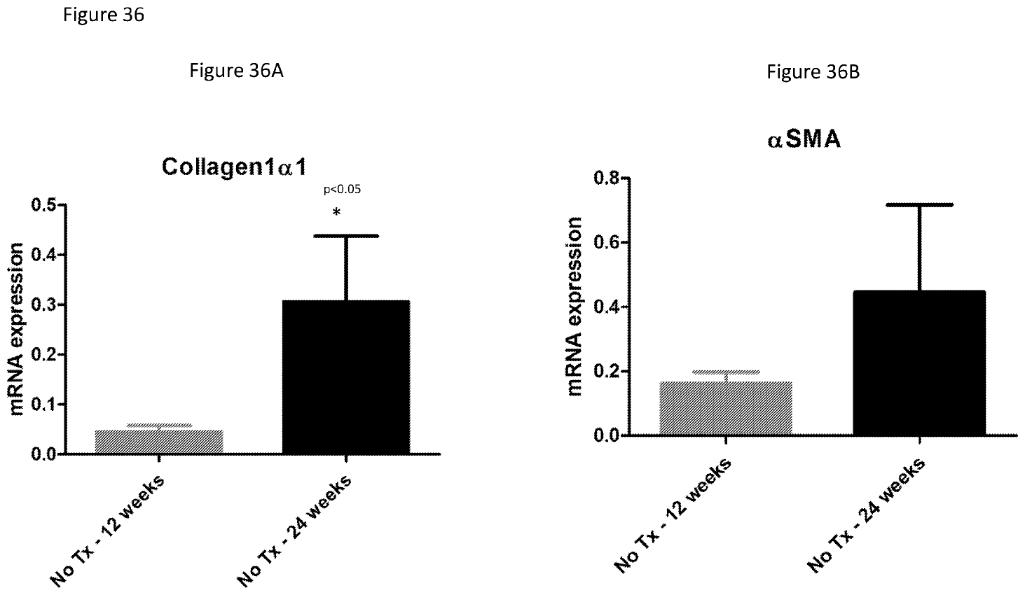

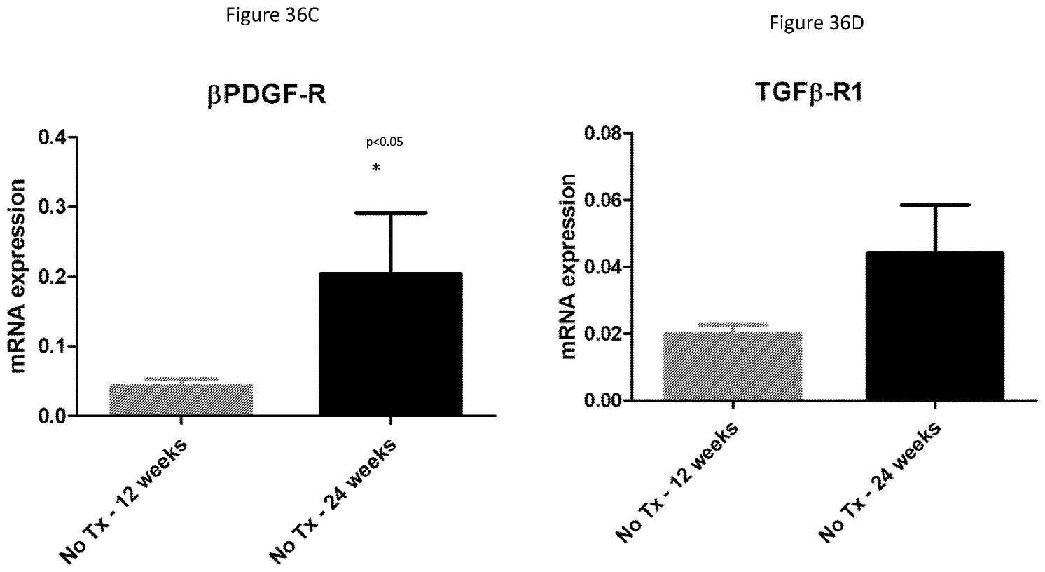

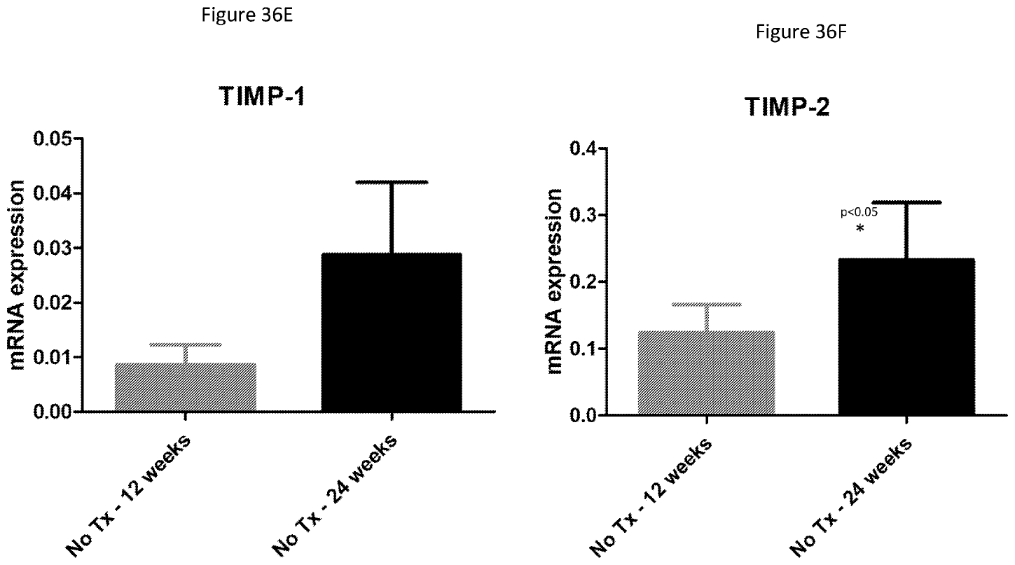

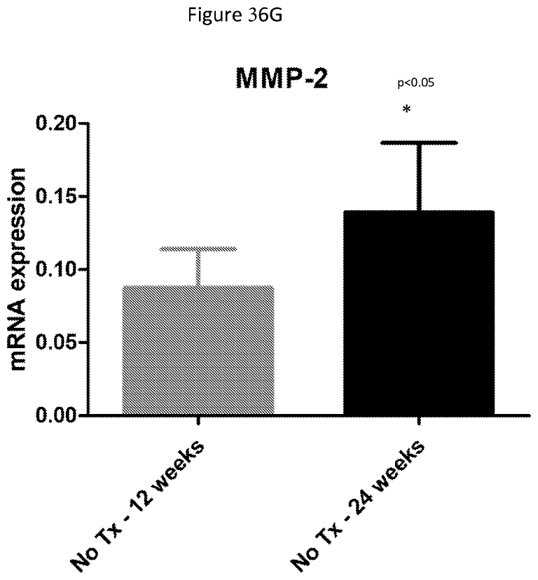

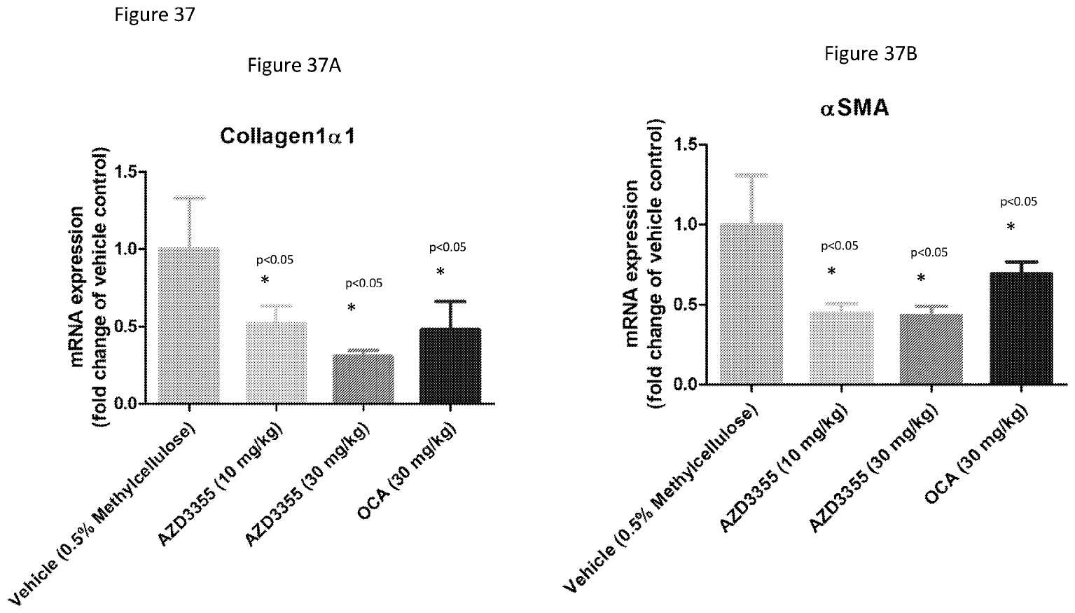

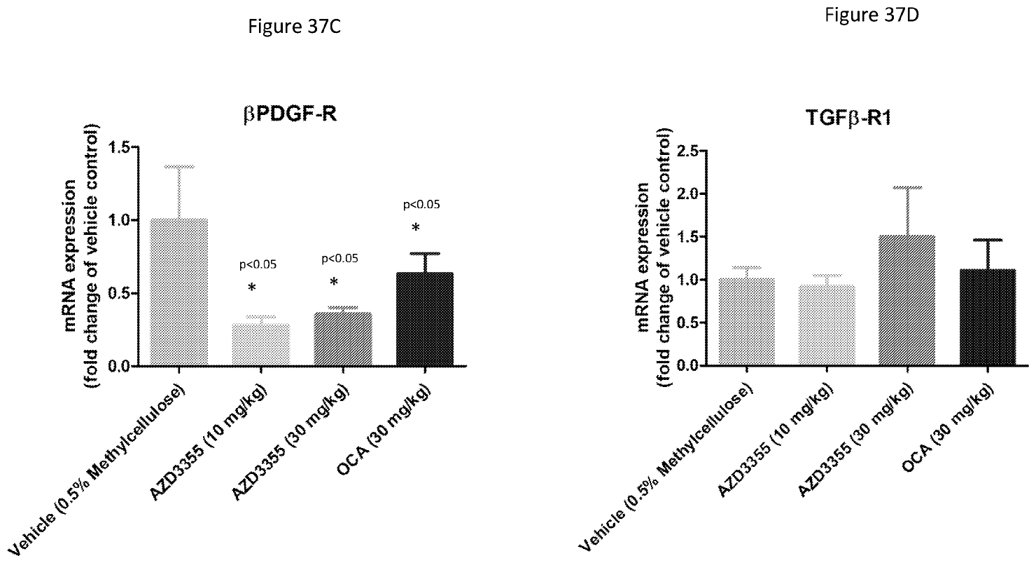

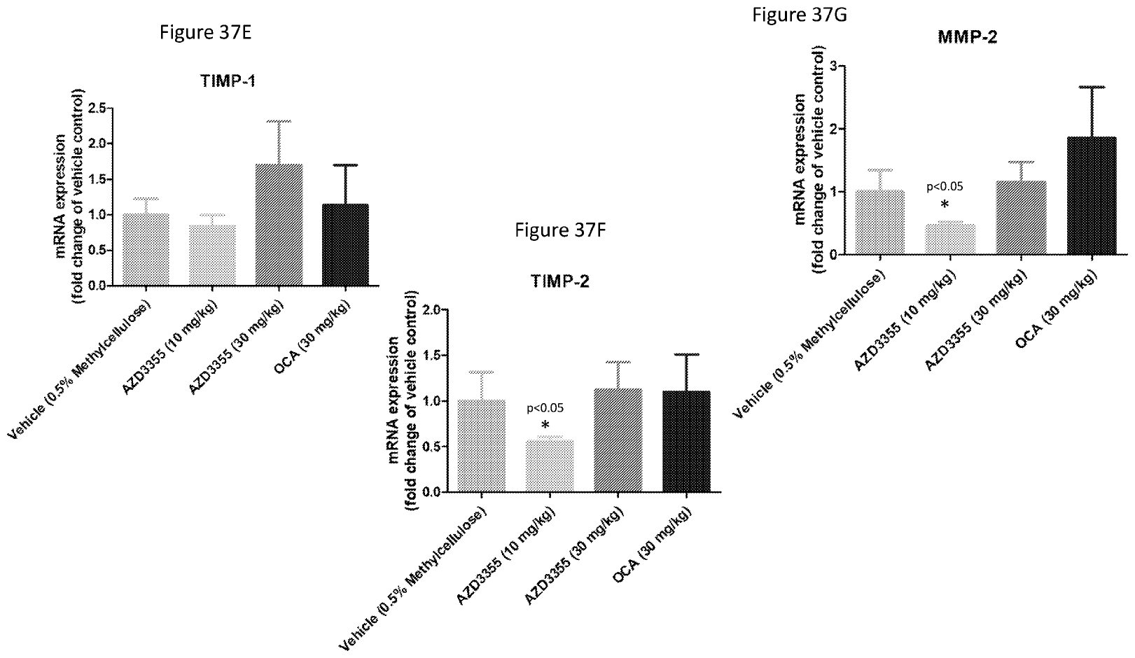

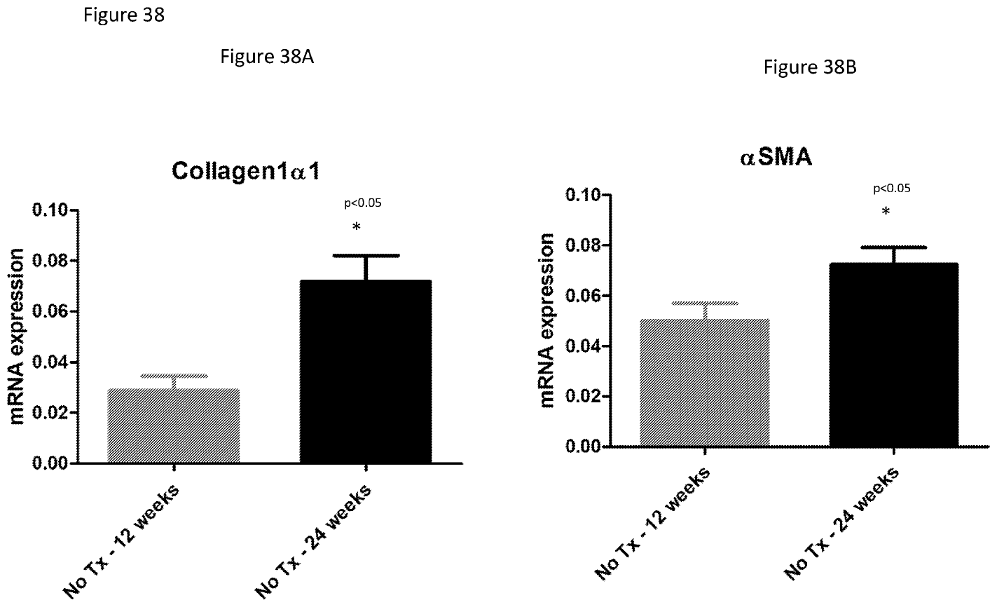

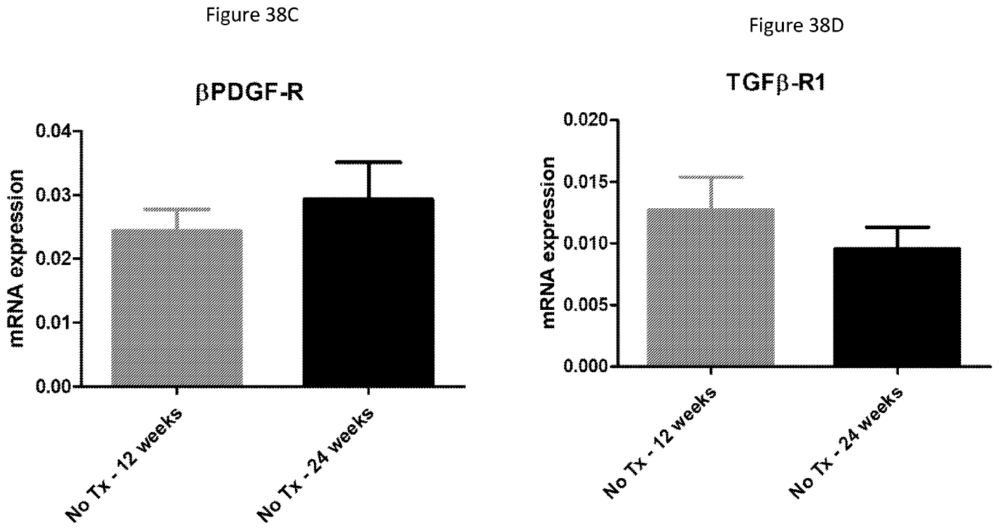

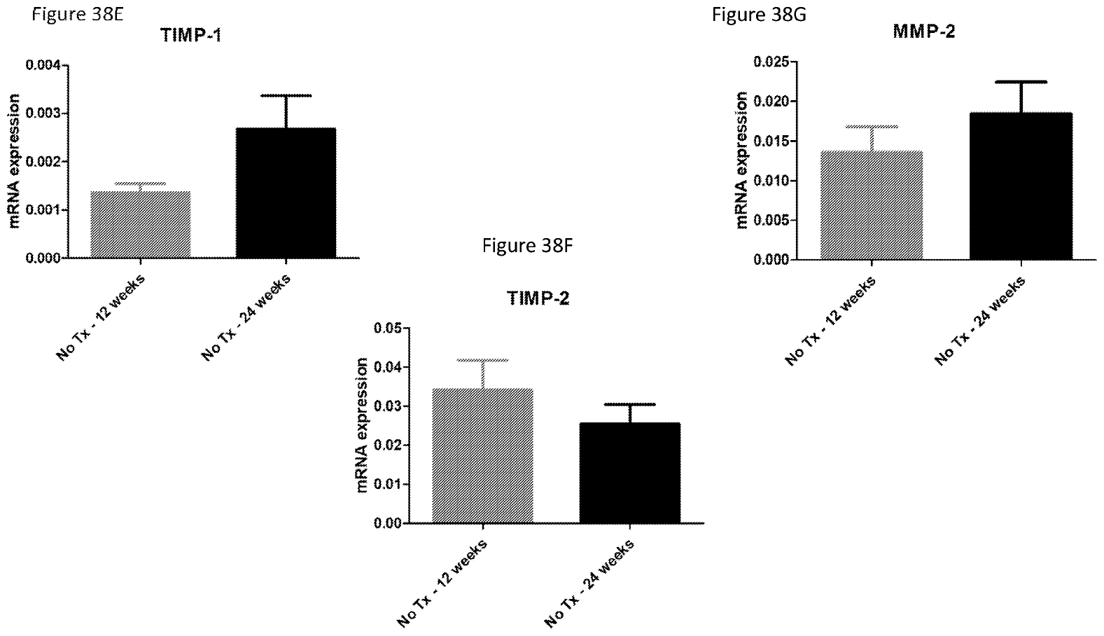

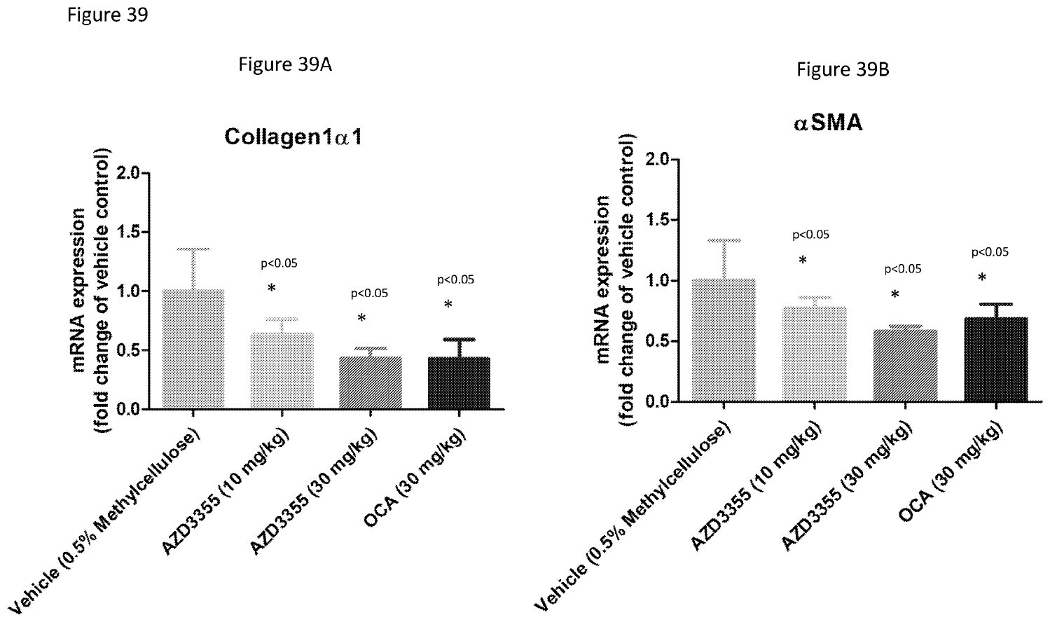

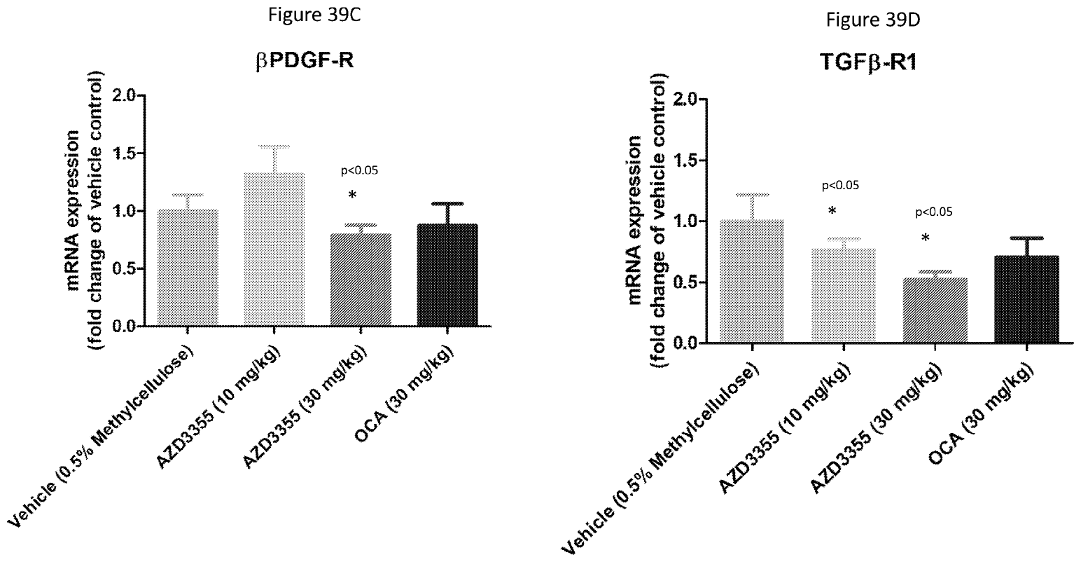

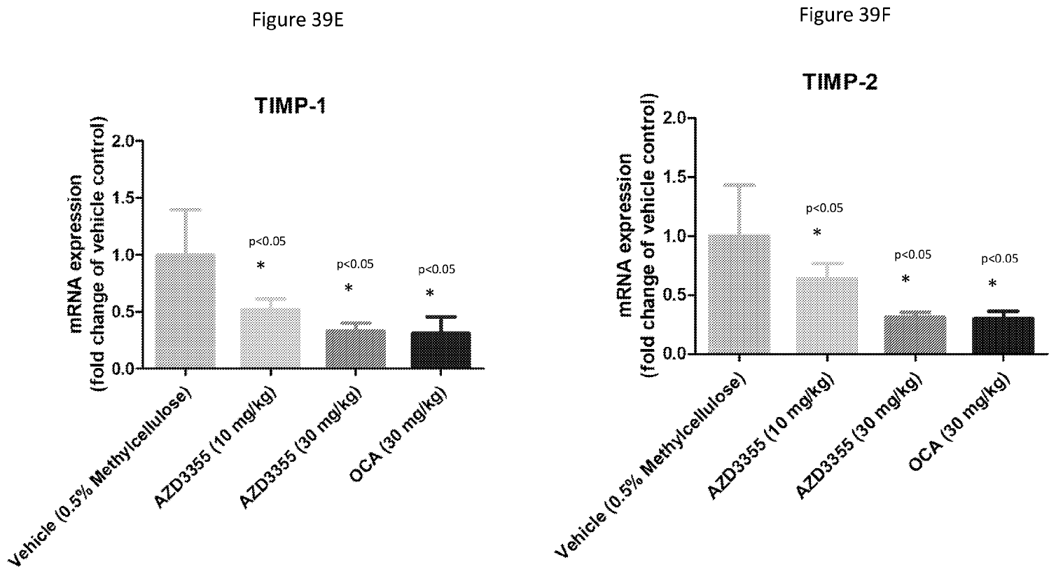

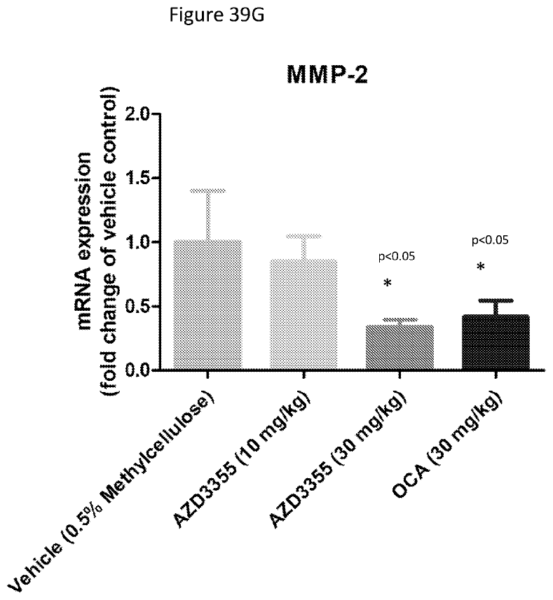

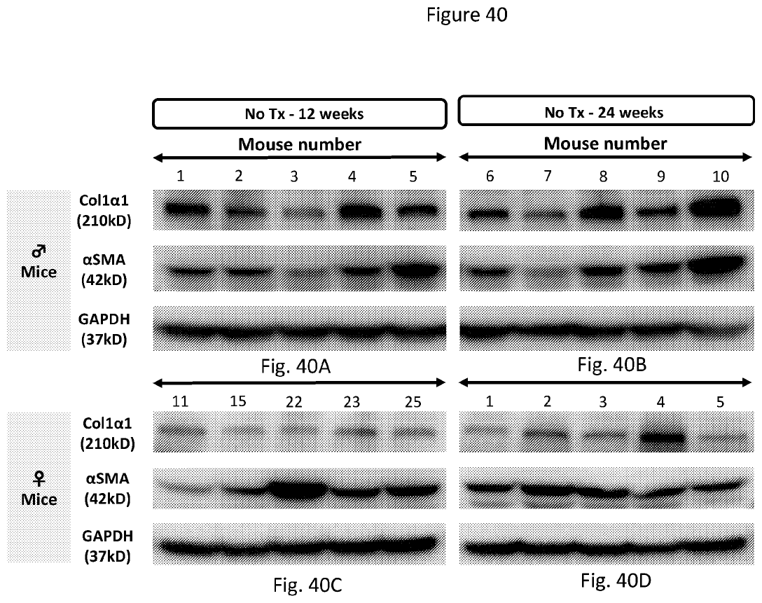

NASH mice also have upregulated profibrotic gene expression at 24 weeks of NASH induction versus 12 weeks. The upregulated genes include Col1α1, αSMA, βPDGF-R, TGFβ-R1, TIMP1, TIMP2, and MMP2. See FIGS. 36 and 38. Treatment with AZD3355 at both dosages and OCA reduced the gene expression of the profibrotic genes. See FIGS. 38 and 40. See also Table 7. GADPH expression (control) did not change across the AZD335 or OCA treatment groups as compared to vehicle treated mice. See FIG. 35.

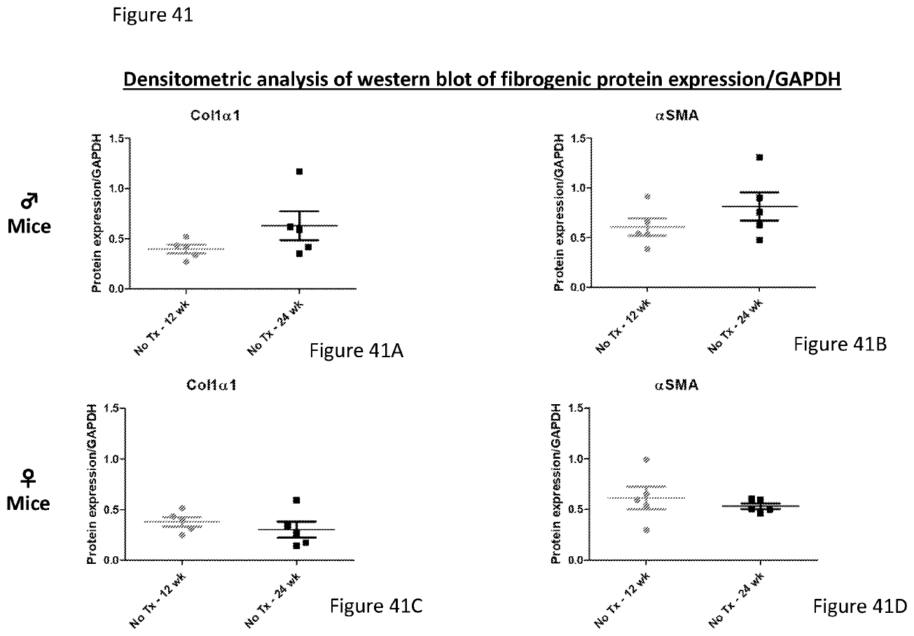

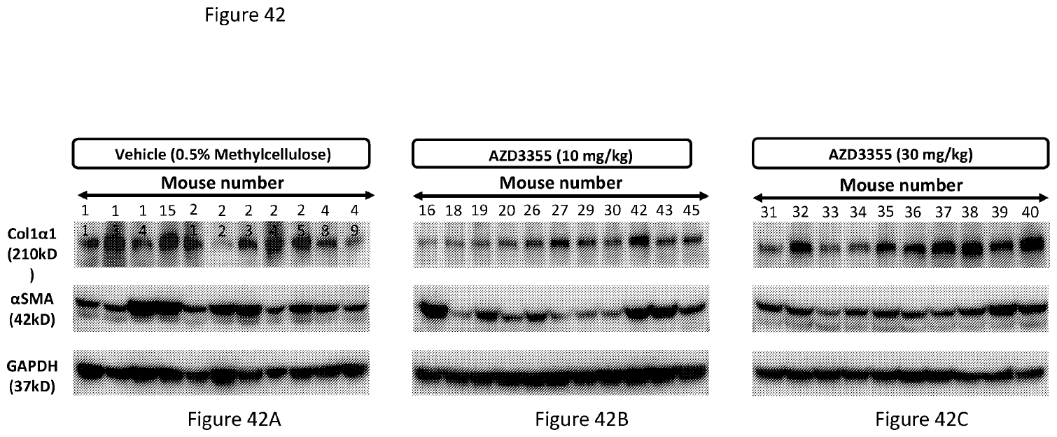

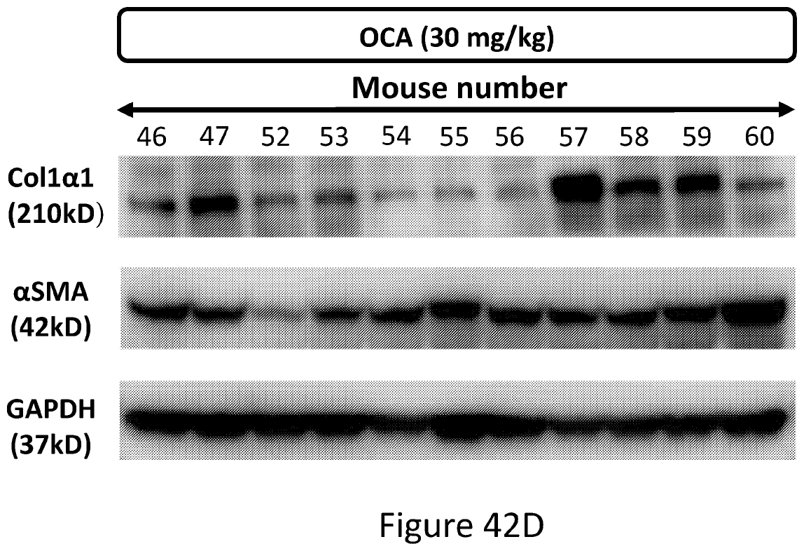

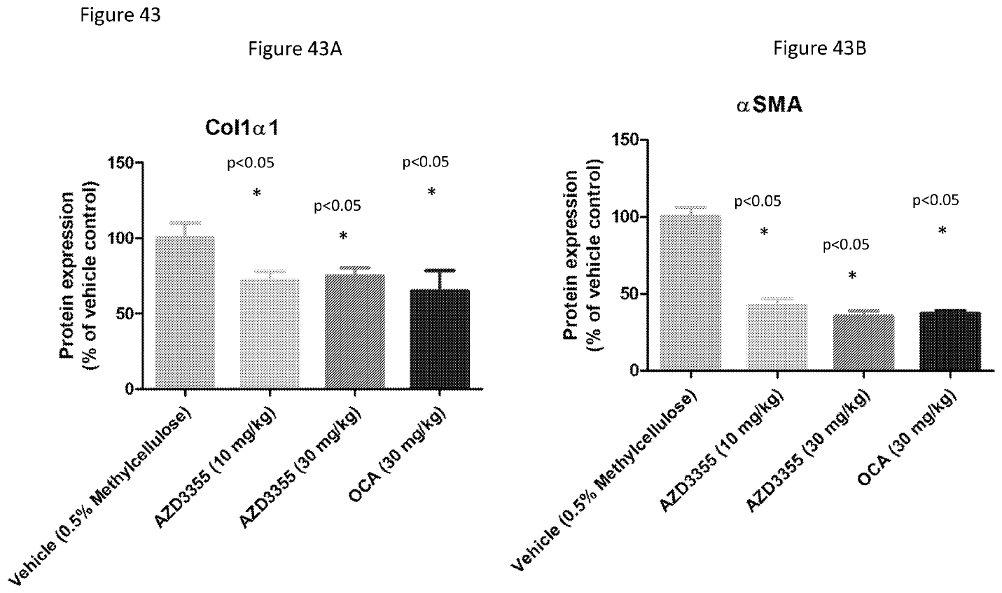

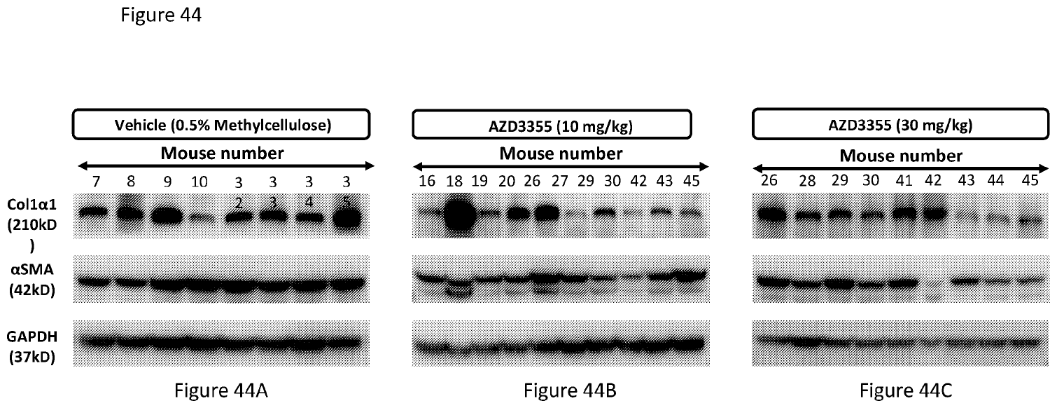

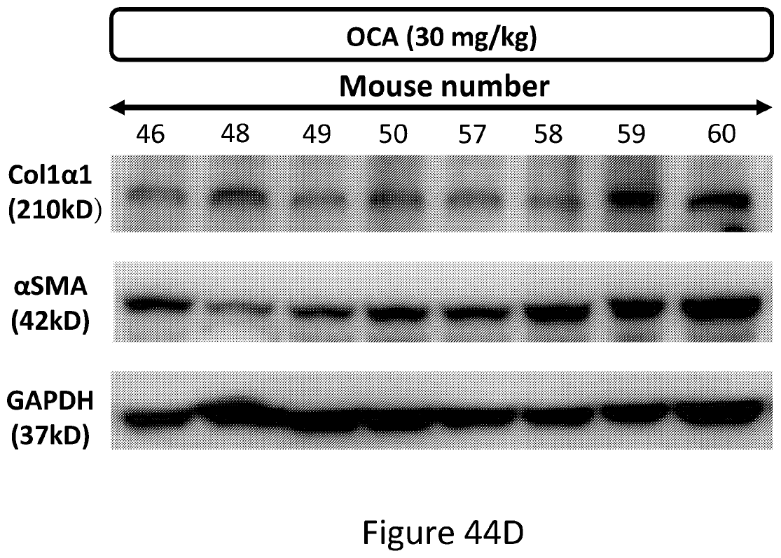

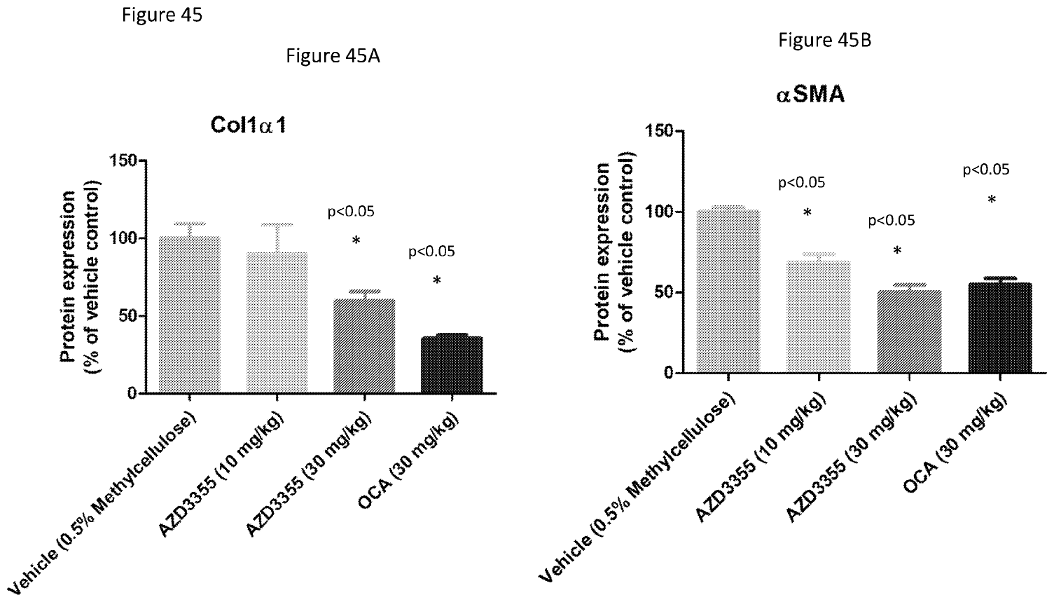

Profibrotic protein expression was also upregulated in the liver of NASH mice at 24 weeks as compared to 12 weeks. See FIGS. 40 and 41. After treatment with AZD3355 or OCA reduced the protein expression of both Col1α1 and αSMA in both male NASH mice (FIGS. 43 and 44 and Table 8) and female NASH mice (FIGS. 44 and 45 and Table 9).

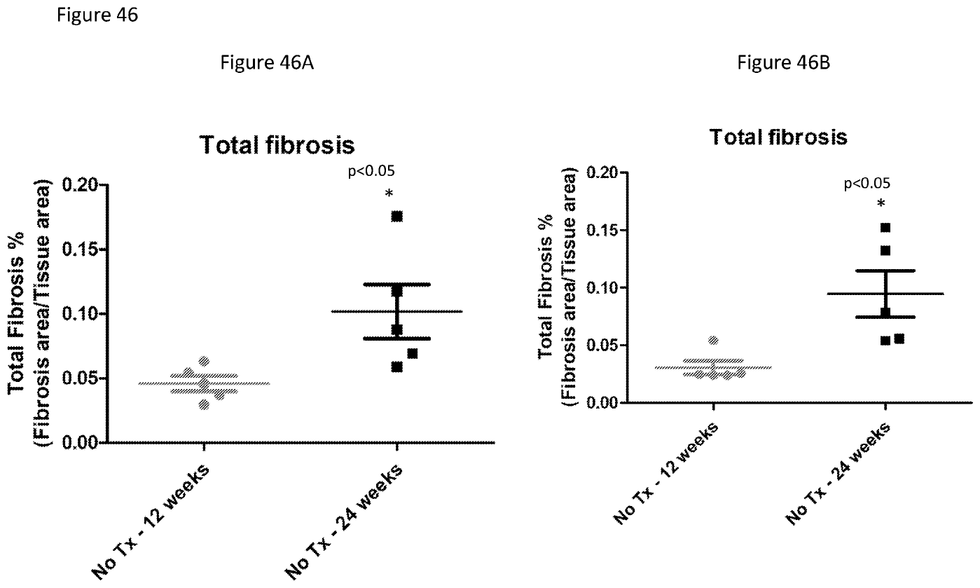

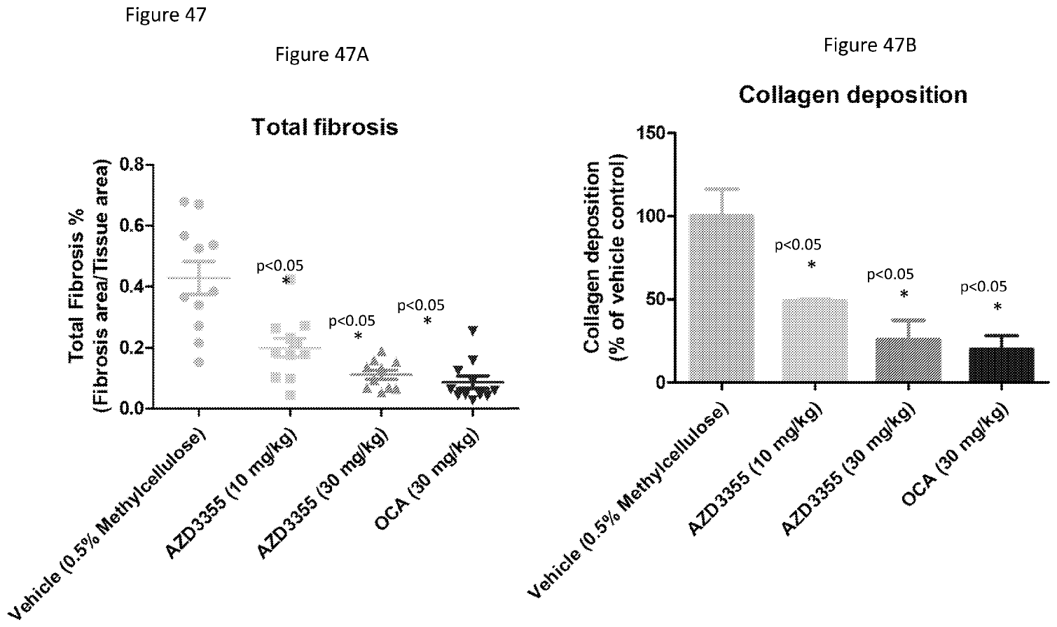

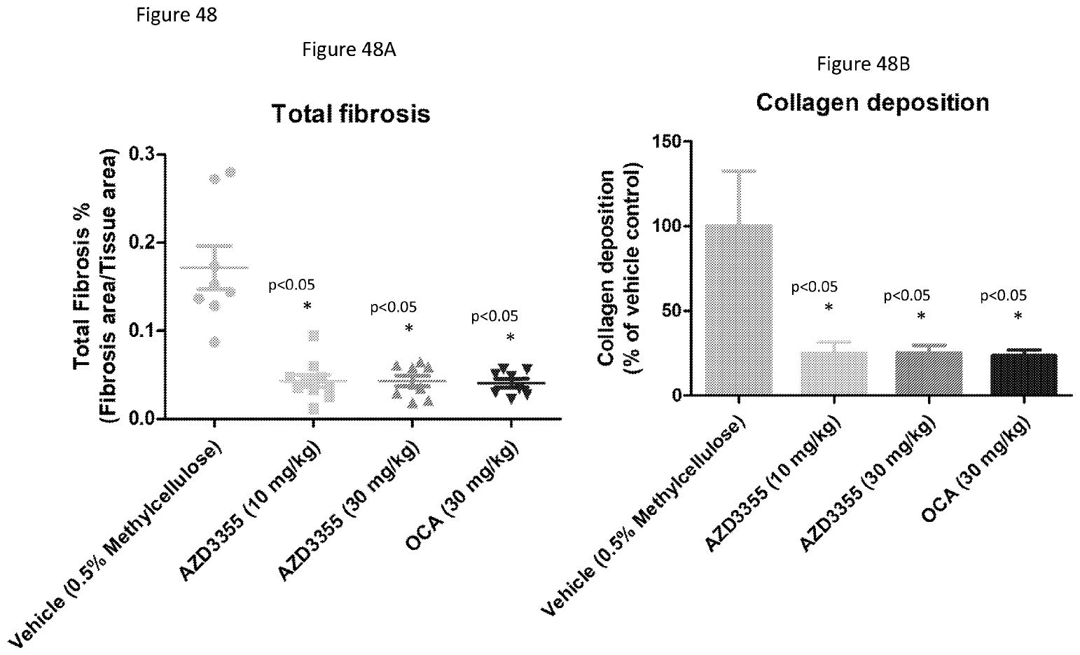

The livers of NASH model mice were stained with picrosirius red/fast green staining for fibrosis to determine hepatic injury and collagen deposition. There was a significant increase in fibrosis in both male NASH model mice and female NASH model mice at 24 weeks as compared to 12 weeks. See FIG. 46. Both total fibrosis and hepatic collagen accumulation were reduced by treatment with AZD3355 and OCA in both male NASH mice (FIG. 47) and female NASH mice (FIG. 48). The reduction in the male mice was dose dependent. See also Table 10.

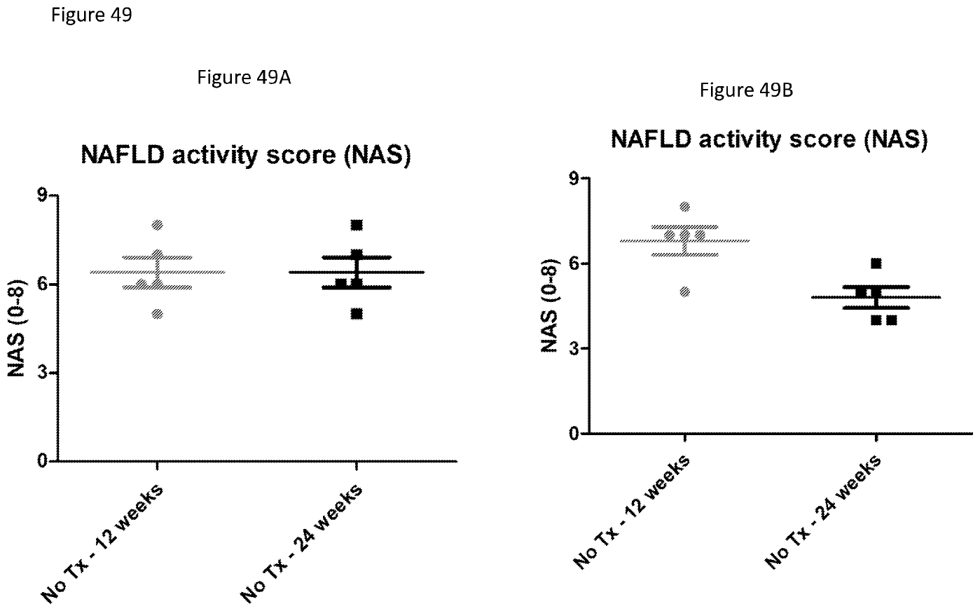

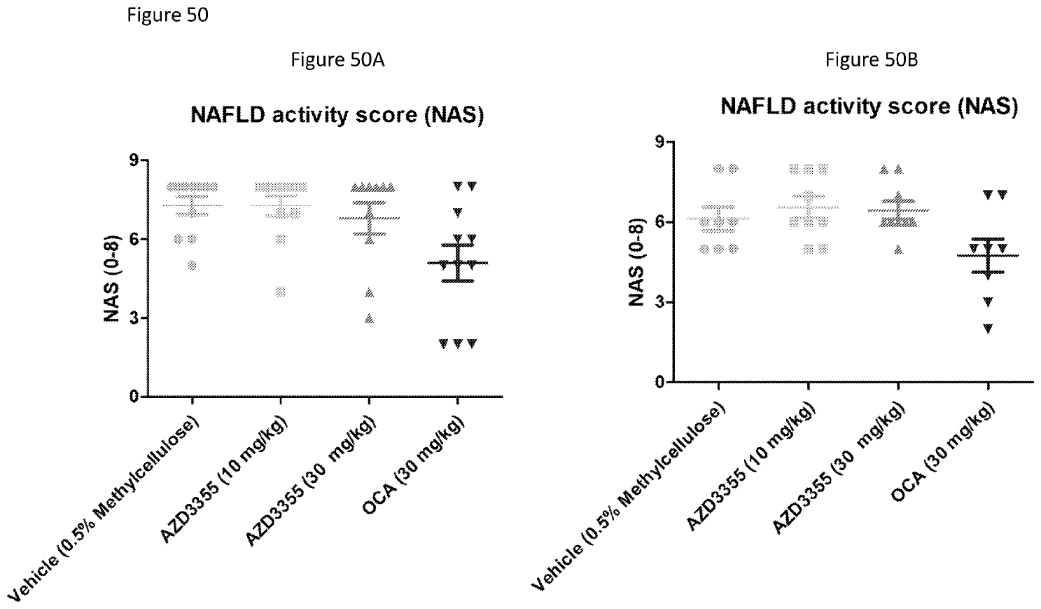

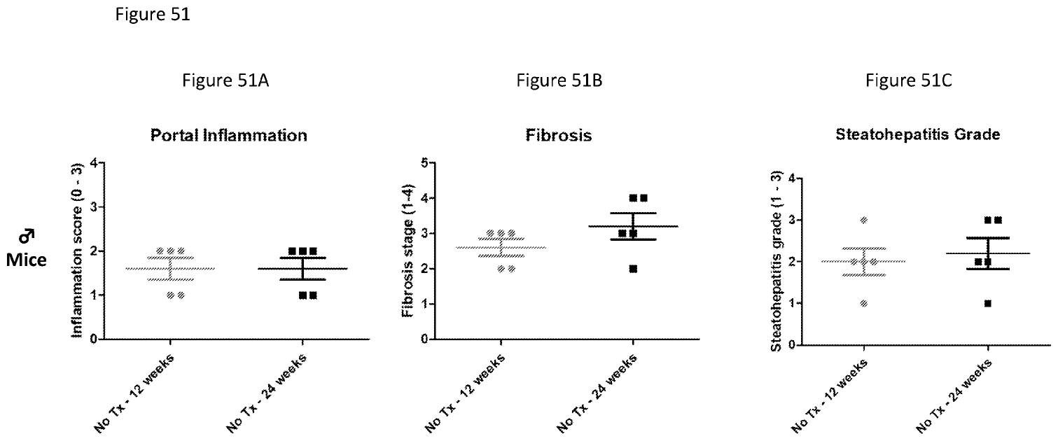

The livers of the NASH mice were assessed for steatosis, hepatocyte ballooning, and lobular inflammation, and the sum of the scores used to calculate a NAFLD activity score (NAS). The NAS of NASH mice at 12 weeks indicated that NASH had been reached in the model mice and was maintained up to 24 weeks. See FIG. 49. Treatment with AZD3355 or OCA indicated towards the reduction of NAFLD activity in the NASH model mice. See FIG. 50.

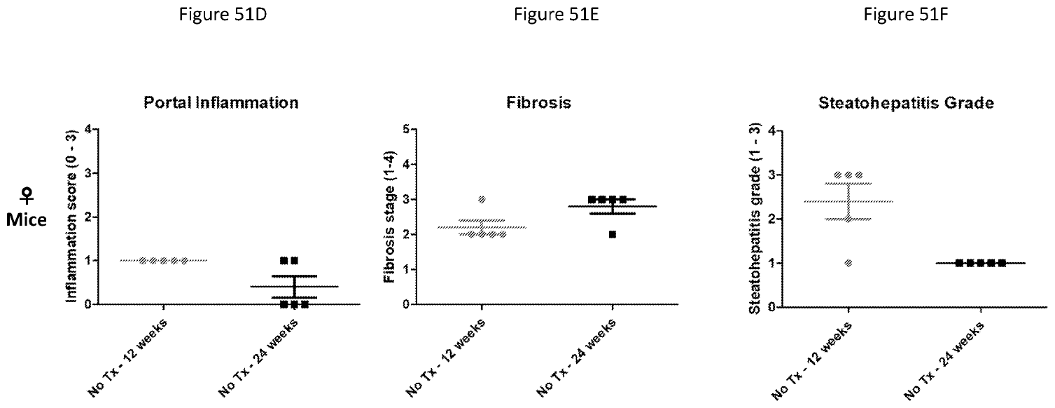

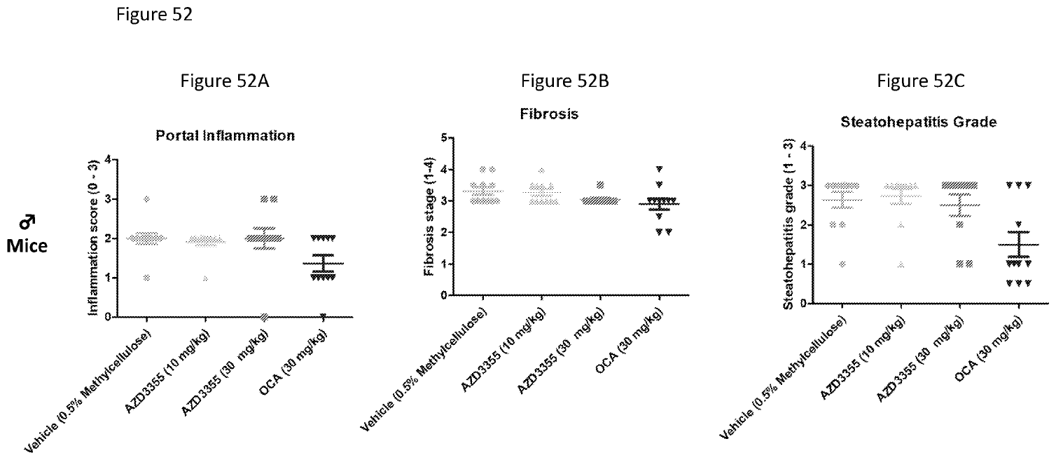

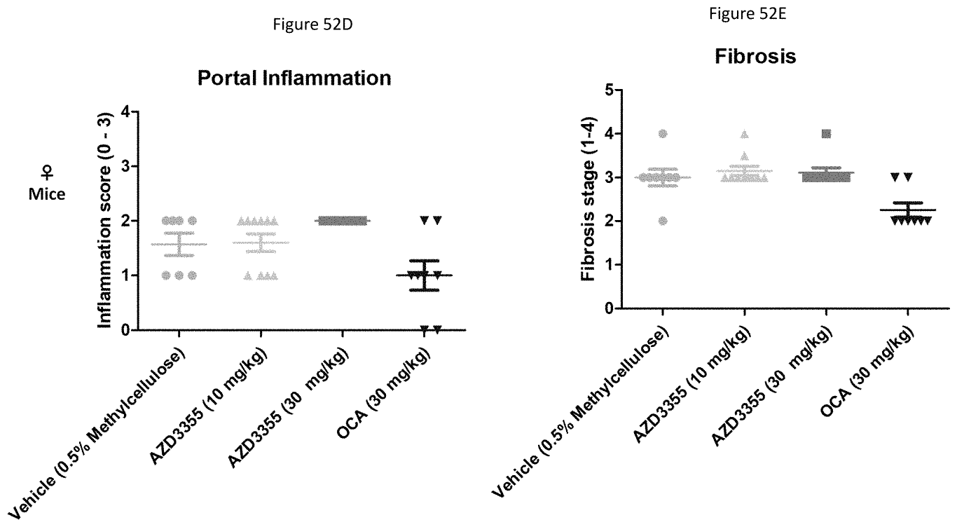

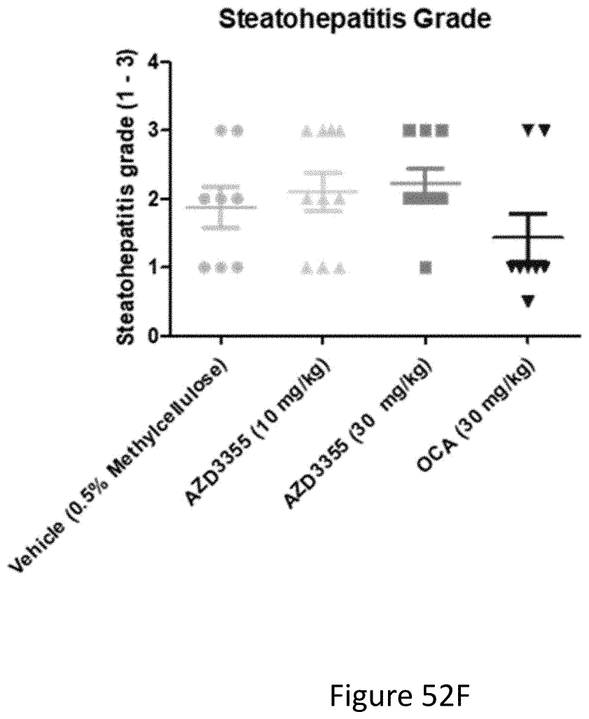

Histopathological scores of portal inflammation was used to assess fibrosis stage and steatohepatitis in the livers of the NASH mice at 12 and 24 weeks. Fibrosis was increased at 24 weeks as compare to 12 weeks indicating significant liver injury in the NASH model mice, both male and female. See FIG. 51. Treatment with either AZD3355 or OCA indicated towards the reduction of the fibrosis and steatohepatitis in both male and female NASH model mice. See FIG. 52 and Table 11.

TABLE 7
expression of pro-fibrogenic genes in NASH model mice after AZD3355 treatment

| | | Col1α1 | αSMA | βPDGF-R | TGFβ-R1 | TIMP-1 | TIMP-2 | MMP-2 |
|---|---|---|---|---|---|---|---|---|
| | | (p value) | (p value) | (p value) | (p value) | (p value) | (p value) | (p value) |
| ♂ | AZD3355 | 48 ↓ | 55 ↓ | 72 ↓ | 8 ↓ | 16 ↓ | 44 ↓ | 53 ↓ |
| Mice | (10 mg/kg) | (<0.05)* | (<0.05)* | (<0.05)* | | | (<0.05)* | (<0.05)* |
| | AZD3355 | 69 ↓ | 55 ↓ | 64 ↓ | 50 ↑ | 70 ↑ | 12 ↑ | 16 ↑ |
| | (30 mg/kg) | (0.05)* | (<0.05)* | (<0.05)* | | | | |
| | OCA | 52 ↓ | 31 ↓ | 37 ↓ | 10 ↑ | 13 ↑ | 10 ↑ | 85 ↑ |
| | (30 mg/kg) | (<0.05)* | (<0.05)* | (<0.05)* | | | | |
| ♀ | AZD3355 | 36 ↓ | 22 ↓ | 32 ↑ | 23 ↓ | 48 ↓ | 36 ↓ | 15 ↓ |
| Mice | (10 mg/kg) | (<0.05)* | (<0.05)* | | (<0.05)* | (<0.05)* | (<0.05)* | |
| | AZD3355 | 57 ↓ | 42 ↓ | 21 ↓ | 48 ↓ | 67 ↓ | 68 ↓ | 66 ↓ |
| | (30 mg/kg) | (<0.05)* | (<0.05)* | (<0.05)* | (<0.05)* | (<0.05)* | (<0.05)* | (<0.05)* |
| | OCA | 57 ↓ | 32 ↓ | 12 ↓ | 29 ↓ | 69 ↓ | 70 ↓ | 58 ↓ |
| | (30 mg/kg) | (<0.05)* | (<0.05)* | | | (<0.05)* | (<0.05)* | (<0.05)* |

TABLE 8
expression of pro-fibrogenic proteins in male
NASH model mice after AZD3355 or OCA treatment

Fibrogenic Protein

| | Col1α1 | αSMA |
|---|---|---|
| Mice group | (p value) | (p value) |
| AZD3355 | 28 ↓ | 57 ↓ |
| (10 mg/kg) | (p < 0.05)* | (p < 0.05)* |
| AZD3355 | 25 ↓ | 65 ↓ |
| (30 mg/kg) | (p < 0.05)* | (p < 0.05)* |
| OCA | 35 ↓ | 62 ↓ |
| (30 mg/kg) | (p < 0.05)* | (p < 0.05)* |

TABLE 9
expression of pro-fibrogenic proteins in female
NASH model mice after AZD3355 or OCA treatment

Fibrogenic protein

| | Col1α1 | αSMA |
|---|---|---|
| Mice group | (p value) | (p value) |
| AZD3355 | 10 ↓ | 31 ↓ |
| (10 mg/kg) | | (p < 0.05)* |
| AZD3355 | 60 ↓ | 50 ↓ |
| (30 mg/kg) | (p < 0.05)* | (p < 0.05)* |
| OCA | 64 ↓ | 45 ↓ |
| (30 mg/kg) | (p < 0.05)* | (p < 0.05)* |

TABLE 10
hepatic collagen accumulation in NASH model
mice after AZD3355 or OCA treatment

| | | % change |
|---|---|---|
| | Mice group | (p value) |
| ♂ Mice | AZD3355 | 51 ↓ |
| | (10 mg/kg) | (<0.05)* |
| | AZD3355 | 74 ↓ |
| | (30 mg/kg) | (<0.05)* |
| | OCA | 80 ↓ |
| | (30 mg/kg) | (<0.05)* |
| ♀ Mice | AZD3355 | 75 ↓ |
| | (10 mg/kg) | (<0.05)* |
| | AZD3355 | 75 ↓ |
| | (30 mg/kg) | (<0.05)* |
| | OCA | 76 ↓ |
| | (30 mg/kg) | (<0.05)* |

TABLE 11
NAS and fibrosis stage in NASH model mice livers treated with vehicle, AZD3355 or OCA

| | | NAS criteria | | | | | | |
|---|---|---|---|---|---|---|---|---|
| | | | Hepatocyte | Lobular | NAFLD activity | Portal | Fibrosis | Steatohepatitis |
| | | Steatosis ± | ballooning ± | inflammation ± | score (NAS) ± | inflammation ± | Stage ± | Grade ± |
| | Mice group | SE | SE | SE | SE | SE | SE | SE |
| ♂ | Vehicle | 2.7 ± 0.1 | 1.7 ± 0.1 | 2.8 ± 0.1 | 7.2 ± 0.3 | 2.0 ± 0.1 | 3.3 ± 0.1 | 2.6 ± 0.2 |
| Mice | (0.5% Meth. cel) | | | | | | | |
| | AZD3355 | 2.7 ± 0.1 | 1.7 ± 0.1 | 2.8 ± 0.1 | 7.2 ± 0.3 | 1.9 ± 0.0 | 3.2 ± 0.1 | 2.7 ± 0.1 |
| | (10 mg/kg) | | | | | | | |
| | AZD3355 | 2.4 ± 0.1 | 1.8 ± 0.1 | 2.6 ± 0.2 | 6.8 ± 0.5 | 2.0 ± 0.2 | 3.0 ± 0.0 | 2.5 ± 0.2 |
| | (30 mg/kg) | | | | | | | |
| | OCA | 2.0 ± 0.2 | 1.0 ± 0.2 | 1.9 ± 0.2 | 5.0 ± 0.6 | 1.3 ± 0.2 | 2.9 ± 0.1 | 1.5 ± 0.3 |
| | (30 mg/kg) | | | | | | | |
| ♀ | Vehicle | 2.5 ± 0.1 | 1.2 ± 0.1 | 2.3 ± 0.1 | 6.1 ± 0.4 | 1.6 ± 0.1 | 3.0 ± 0.1 | 1.8 ± 0.2 |
| Mice | (0.5% Meth. cel) | | | | | | | |
| | AZD3355 | 2.6 ± 0.1 | 1.3 ± 0.1 | 2.5 ± 0.1 | 6.4 ± 0.4 | 1.6 ± 0.1 | 3.1 ± 0.1 | 2.1 ± 0.2 |
| | (10 mg/kg) | | | | | | | |
| | AZD3355 | 2.3 ± 0.1 | 1.3 ± 0.1 | 2.7 ± 0.1 | 6.4 ± 0.3 | 2.0 ± 0.0 | 3.1 ± 0.1 | 2.2 ± 0.2 |
| | (30 mg/kg) | | | | | | | |
| | OCA | 2.0 ± 0.2 | 1.1 ± 0.2 | 1.6 ± 0.1 | 4.7 ± 0.6 | 1.0 ± 0.2 | 2.2 ± 0.1 | 1.4 ± 0.3 |
| | (30 mg/kg) | | | | | | | |

### REFERENCES

Ascha et al. (2010) Hepatology 51(6):1972-1978
Ahrens, et al. (2013) DNA methylation analysis in nonalcoholic fatty liver disease suggests distinct disease-specific and remodeling signatures after bariatric surgery. Cell Metab. 2013; 18(2):296-302.
Alstermark, et al. (2008). “Synthesis and Pharmacological Evaluation of Novel γ-Aminobutyric Acid Type B (GABAB) Receptor Agonists as Gastroesophageal Reflux Inhibitors”. Journal of Medicinal Chemistry 51 (14): 4315-4320.
Bettler, et al. (2004) Physiol Rev. 84(3):835-67.
Bredenoord (2009). “Lesogaberan, a GABA_{B} agonist for the potential treatment of gastroesophageal reflux disease”. IDrugs 12 (9): 576-584.
Dodds, et al. (1982) N Engl J Med. 307(25):1547-52.
Fan, et al., (2013) γ-Aminobutyric Acid B Receptor Improves Carbon Tetrachloride-Induced Liver Fibrosis in Rats.” Digestive diseases and sciences 58.7: 1909-1915.
Hudgson and Weightman (1971) Baclofen in the treatment of spasticity. Br Med J. 4(5778):15-7.
Lacy, et al. (2010). “Lesogaberan”. Drugs of the Future 35 (12): 987-992.
Lamb, et al. (2006) The Connectivity Map: using gene-expression signatures to connect small molecules, genes, and disease. Science 313(5795):1929-35.
Lee, et al. (2105) Pathobiology of liver fibrosis: a translational success story. Gut 64.5 (2015): 830-841.
Liu, et al. (2011). Upregulation of hemoglobin expression by oxidative stress in hepatocytes and its implication in nonalcoholic steatohepatitis. PLoS One, 6(9), p.e24363.
Marengo, et al. (2015) Liver Cancer: Connections with Obesity, Fatty Liver, and Cirrhosis. Annu Rev Med. doi: 10.1146/annurev-med-090514-013832. PubMed PMID: 26473416.
Wang, et al., (2008) Baclofen, a GABAB receptor agonist, inhibits human hepatocellular carcinoma cell growth in vitro and in vivo. Life sciences 82.9: 536-541.

All references cited herein are incorporated by reference to the same extent as if each individual publication, database entry (e.g. Genbank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

The foregoing written specification is considered to be sufficient to enable one skilled in the art to practice the invention. Various modifications of the invention in addition to those shown and described herein will become apparent to those skilled in the art from the foregoing description and fall within the scope of the appended claims.

Many modifications and variations of this invention can be made without departing from its spirit and scope, as will be apparent to those skilled in the art. The invention is defined by the terms of the appended claims, along with the full scope of equivalents to which such claims are entitled. The specific embodiments described herein, including the examples, are offered by way of example only, and do not by their details limit the scope of the invention.

1. A method for treating or preventing a liver disease or condition in a subject in need thereof, comprising administering a therapeutically effective amount of a peripheral acting GABA_{B} agonist or a pharmaceutically acceptable salt thereof.

2. The method of claim 1, wherein the liver disease or condition is selected from the group consisting of fatty liver disease, nonalcoholic fatty liver disease, adiposity, liver fibrosis, cirrhosis, hepatocellular carcinoma, and combinations thereof.

3. The method of claim 2, wherein the fatty liver disease is steatosis hepatitis or steatohepatitis.

4. The method of claim 2, wherein the nonalcoholic fatty liver disease is nonalcoholic steatosis hepatitis or nonalcoholic steatohepatitis (NASH).

5. The method of claim 2, wherein the liver fibrosis is associated with or due to fatty liver disease, nonalcoholic fatty liver disease, liver inflammation, hepatocyte injury or death, adiposity, hepatocellular carcinoma, and any combination thereof.

6. (canceled)

7. (canceled)

8. The method of claim 1, wherein the GABA_{B} agonist is AZD3355, or a pharmaceutically acceptable salt thereof.

9. The method of claim 1, wherein the GABA_{B} agonist or a pharmaceutically acceptable salt thereof is in a pharmaceutical composition further comprising a pharmaceutically acceptable carrier.

10. The method of claim 1, wherein the GABA_{B} agonist or a pharmaceutically acceptable salt thereof is administered twice daily.

11. (canceled)

12. The method of claim 1, wherein the subject is a human.

13. The method of claim 1, wherein the subject has one or more symptoms selected from the group consisting of hepatic inflammation, hepatocyte injury or death, insulin resistance, weight gain, dyslipidemia, and fibrosis, and the administration of the peripheral acting GABA_{B} agonist decreases one or more of the symptoms in the subject.

14. A method of inhibiting liver fibrosis in a subject in need thereof, comprising administering a therapeutically effective amount of a peripheral acting GABA_{B} agonist, or a pharmaceutically acceptable salt thereof.

15. The method of claim 14, wherein the liver fibrosis is associated with nonalcoholic steatosis hepatitis, nonalcoholic steatohepatitis (NASH), steatosis hepatitis or steatohepatitis.

16. (canceled)

17. The method of claim 14, wherein liver fibrosis is associated with or due to fatty liver disease, adiposity, liver inflammation, hepatocyte injury or death, hepatocellular carcinoma, and combinations thereof.

18. The method of claim 14, wherein the liver fibrosis is caused by alcohol use, an infection, or an immune mediated disorder.

19. (canceled)

20. The method of claim 14, wherein the GABA_{B} agonist is AZD3355, or a pharmaceutically acceptable salt thereof.

21. The method of claim 14, wherein the GABA_{B} agonist or a pharmaceutically acceptable salt thereof is in a pharmaceutical composition further comprising a pharmaceutically acceptable carrier.

22. The method of claim 14, wherein the GABA_{B} agonist or a pharmaceutically acceptable salt thereof is administered twice daily.

23. (canceled)

24. The method of claim 14, wherein the subject is a human.

25. The method of claim 14, wherein the subject has one or more symptoms selected from the group consisting of hepatic inflammation, hepatocyte injury or death, insulin resistance, weight gain, dyslipidemia, and fibrosis, and the administration of the peripheral acting GABA_{B} agonist decreases one or more of the symptoms in the subject.

26. A method for increasing GABA_{B} activity in a hepatocyte comprising contacting the hepatocyte with AZD3355, or a pharmaceutically acceptable salt thereof.
